SEQUENCE LISTING (32 sequences)

SEQ ID NO: 1 - DNA, 531 bp, Dekkera anomalus
atgcagcttccttgctatcaaaacaatacaccacttgacccttcatttgaacatgatatgagggacgttcatcttgtttatgattacaacgctaaggattctgatggaaagccagaaaagtggagatatgaaatttggttcttttcagaaagcagggttgtctatgcaattcatggaggacctatggccggaagaataaactatcagactgctgcttatcagtgtgtgcgtcctggagagatatggcaggtcaattggctcgaagaaacgggtactgtcgtttcattggtttatgacattgtaaatgagactgttaacggtcttctatgtttttctaaaggacattgggaaaatgcagaagctgctcacggtgataagagaaatcctgaagattttgcacgttggaaagcattagcttcacagggaatgcagacagatcgttttgttttggttgaaagagcccatatattagaaaggtttaaaggtaagggcgatttggttccaattgagcctgacgctatcaccttttaa

SEQ ID NO: 2 - protein, 176 aa, Dekkera anomalus
MQLPCYQNNTPLDPSFEHDMRDVHLVYDYNAKDSDGKPEKWRYEIWFFSESRVVYAIHGGPMAGRINYQTAAYQCVRPGEIWQVNWLEETGTVVSLVYDIVNETVNGLLCFSKGHWENAEAAHGDKRNPEDFARWKALASQGMQTDRFVLVERAHILERFKGKGDLVPIEPDAITF

SEQ ID NO: 3 - DNA, 465 bp, Dekkera anomalus
atggttaaagcagttgcagttgttagaggagactctacagttaagggagtcgtcacctttgagcagacctccgaggatgagccaactaccattaactacaacatcgaaggtaatgatccaaacgctttgagaggattccacattcacactttcggtgacaataccaacggttgtacttcggcaggtccacacttcaacccatttggaaagacccacggtgctccaaccgacgaggtcagacatgttggtgacttgggaaacatcaagactgatgctaacggagttgccaaaggtactatcaaggacaagcttgtgaagcttatcggtgagaactctatcatcggaagaaccgttgttgtccatgctggaactgatgatttgggtaaaggtggtgatgctggctctttgcagaccggtaatgccggtggcagaccagcttgtggtgtcattggattgtccgcttaa

SEQ ID NO: 4 - protein, 154 aa, Dekkera anomalus
MVKAVAVVRGDSTVKGVVTFEQTSEDEPTTINYNIEGNDPNALRGFHIHTFGDNTNGCTSAGPHFNPFGKTHGAPTDEVRHVGDLGNIKTDANGVAKGTIKDKLVKLIGENSIIGRTVVVHAGTDDLGKGGDAGSLQTGNAGGRPACGVIGLSA

SEQ ID NO: 5 - DNA, 531 bp, Dekkera bruxellensis
atgaaactcccttgctatcaaaacaatacgccccttgatccttccttcgatgatgacctgaaggacgttcatctcgtctatgattatgatgccacagattcgaacggaagaccagaaaaatggaggtatgaaatatggtttttctcagaaaataaaattgtttatgcgattcatggtggtccaatggcaggaaggattaattatcaaacggttgcttatcaatgtgtacgccctggagaaatatggcagataaattggcttgaagaaacaggcactgttgtgtcaatagtttatgacattgtgaataaaacggtaaacggacttctatgcttttctaagggacattgggaaaattctgaagctgctcatggggataaaagaaacccagatgactttgcccgttggagaaatttggccaagcagggcattcaaaccgatcgtttcgtcttggttgaaagagcccatatattgaaatcatttaaaggtcagggtgatttggaaccgatcgaacctgatgcaattaccttttag

SEQ ID NO: 6 - protein, 176 aa, Dekkera bruxellensis
MKLPCYQNNTPLDPSFDDDLKDVHLVYDYDATDSNGRPEKWRYEIWFFSENKIVYAIHGGPMAGRINYQTVAYQCVRPGEIWQINWLEETGTVVSIVYDIVNKTVNGLLCFSKGHWENSEAAHGDKRNPDDFARWRNLAKQGIQTDRFVLVERAHILKSFKGQGDLEPIEPDAITF

SEQ ID NO: 7 - DNA, 465 bp, Dekkera bruxellensis
atggttaaagcagttgcagttgtcagaggagactctacagttaaaggtgttgttacctttgagcaaacctcagagagtgagccaacaactattaactacaacatcgagggtaacgatccgaatgctttgagaggattccacattcacacatttggcgacaacaccaacggttgcacatcagctggtcctcacttcaacccattcggtaagactcatggtgctccaaccgacgaaaacaggcacgttggtgatttgggtaacatcaagacggatgccaatggagttgctaagggcaccatcaaggacaagttagttaagcttattggtgcgaactccatcattggaagaaccgtcgttgttcacgctggaactgacgatttgggtaagggcggtgatgctggttctttgcagactggtaacgccggtggcagaccagcatgtggtgtcattggcttgtctgcataa

SEQ ID NO: 8 - protein, 154 aa, Dekkera bruxellensis
MVKAVAVVRGDSTVKGVVTFEQTSESEPTTINYNIEGNDPNALRGFHIHTFGDNTNGCTSAGPHFNPFGKTHGAPTDENRHVGDLGNIKTDANGVAKGTIKDKLVKLIGANSIIGRTVVVHAGTDDLGKGGDAGSLQTGNAGGRPACGVIGLSA

SEQ ID NO: 9 - DNA, 1770 bp, Dekkera anomalus
atgagcgcaatcgaagataacaatagctctgtctcacagcttgatgaagctaaggcagataacgagcacatcgaaaactctgctccaaatgatgtcgatgatgagattatcactcgtttccttggcgcttctgatgaagtgaaggaaggtgagaagcaagaaaaggagatgggcttcaaggctgccatgaaagcatatccaaagtgtgctgcatggtctgttgtcttatcmagtggacttatcatggaaggttacgatacctgtttgttgagttctatgtatgctctcccaatcttctgtgaacgttttggtacatacgatacgagctcgaaggtytgggaratyastgcaarrtggcagattggtttaagtatgtgtgttaactgtggtgaaatcattggtttgcagatgtctggttggtttgcagacagatttggttayagatatacmttgattgtttctatgttcttcctcgcagcattcacgttcattattttctttgctagaaatattgctatgattgctgttggtcagattctatgtggtattccatggggattcttccagacgttggctgtttcttatgcttcggaggtttgtccgttagctttgagatactaccttaccagttatgtgaatgtttgctgggtgtttggacagttgtttgcctcaggtatcatgaagaactctcaggaaagattggaacattctaatatgggatggagacttccatttggtcttatgtggatctggcccgttcctattgctcttggaatcttcttagcaccggaatctccatggtggctcgttaaaaaaaatagactttcgcaggcagcaaacagtttgagaagacttctttctatcaaggatcacgatcagaaagagatggttgttgatacgatggtcaagagaatgaagtttactggtgataaggaagctagtaagggtgaagagattggttcatactgggaatgcttccacaagaagcactggagaagaactagaatcgctagtatggtttggattggacaaaatcttactggttcgacattgatgggatactcgacatacttttatgaaaaggcaggattatctacgtcatactcatttgacttcagtattattcaatactgtcttggtattgtcggtgctgtgatctcatggattttatctaactctgttggacgtgccacaatttacattggtggtatgggtattcaaacaatcattttgtggctcacaggtattctcggttttgttcatcaaacaactgctattggttgggccgttggatcgttgttgttggtcttcactttggtctacgacagcactgtcggaccagttacctactgtattgttcctgagattccatctggtaaggttagaactaagactgttgtcatttcaagaaatctttacaacattgccggtatctttaatgctgttattactccataccagttgaactctacacagtggaactggggtgccaagagtggtattttctgggcttgctttgccacgttcttcttggcatggtccatttgggatcttccggagaccaagggcagaacctttgcagatattgaccagttgttcgaagaacatattccagcaagaaagttcaagagcactgtcgttgatccattcaacaacgccaagatgcttcagcagttcgacaagaacaaacttaacgagcttggaaacatcaaagctgaagatgaaggaactgaaagttctgacatcgagaagaatggctttactaactga

SEQ ID NO: 10 - protein, 589 aa, Dekkera anomalus
  misc_feature (116)..(116): Xaa can be any naturally occurring amino acid
  misc_feature (118)..(118): Xaa can be any naturally occurring amino acid
MSAIEDNNSSVSQLDEAKADNEHIENSAPNDVDDEIITRFLGASDEVKEGEKQEKEMGFKAAMKAYPKCAAWSVVLSSGLIMEGYDTCLLSSMYALPIFCERFGTYDTSSKVWEIXAXWQIGLSMCVNCGEIIGLQMSGWFADRFGYRYTLIVSMFFLAAFTFIIFFARNIAMIAVGQILCGIPWGFFQTLAVSYASEVCPLALRYYLTSYVNVCWVFGQLFASGIMKNSQERLEHSNMGWRLPFGLMWIWPVPIALGIFLAPESPWWLVKKNRLSQAANSLRRLLSIKDHDQKEMVVDTMVKRMKFTGDKEASKGEEIGSYWECFHKKHWRRTRIASMVWIGQNLTGSTLMGYSTYFYEKAGLSTSYSFDFSIIQYCLGIVGAVISWILSNSVGRATIYIGGMGIQTIILWLTGILGFVHQTTAIGWAVGSLLLVFTLVYDSTVGPVTYCIVPEIPSGKVRTKTVVISRNLYNIAGIFNAVITPYQLNSTQWNWGAKSGIFWACFATFFLAWSIWDLPETKGRTFADIDQLFEEHIPARKFKSTVVDPFNNAKMLQQFDKNKLNELGNIKAEDEGTESSDIEKNGFTN

SEQ ID NO: 11 - DNA, 1761 bp, Dekkera anomalus
atgacttcagaattagcaaaacatccagagactaagccacaatggtggaaggaagcaaccttctatcaaatctatccagcatcttttaaagattctaataatgatggttggggagacatcaacggtatcagatccaagctttcatatttgaagagtcttggtatcacagcagtttggatttctcctttctatgactcgcctcaaaaagatatgggatacgatatcagagattatgacaaggtttggccaacatatggaacgaacgaagaaatatttcacttgatcaaggaggcccacaaacttgatatcaggattgtggtcgatttggttattaaccactgctcggatgagcacaaatggtttgaggagtccagatcaagtcttgataatccaaagagggattggttttactggagaaagccaaaaggttatactaaagatggtaagccaatacctccaaataactggagatcattttttggtggatctgcatgggaatatgatgaaagaactaaagaattttatttgcatttgtttgcagttggacagccagatctaaattgggagaatccagtgtgtaggtcagctatctttgaagattctatgggattctggttgagaaatggtgtcgatggtttcagaattgataccgccttcctttactctaaggacactaccttccctgatgcccctattaccaaccctaaggatgattttcaagatccaggtaactttgtttcaaacggtcctagaattcatgaattccacaagttgatgcaccacttcatgctttcccagattgatgacaagagaactattatgacagttggtgaggttggtcatgcaggtaacgatgccagggtggcatataccagctcagcttctcacgaattgtctgaaattttcacatttaaacacaccgacgccggaacatctgaaaagttcagatacgacttggttccattcacattgaaagattggaagttagctgttgctgactccttccgttggattgatggtactgattcatggtcaacgatttactttgagaatcatgaccaggctagatctgtttctagatttactgatgattcacctgagtcacgtgttcctgcagctaaaatgcttgcctctctccagatctgtatgaccggaacaatgtatgtctatcagggtcaagagttaggttgtatcaacttgaaaaacactcctctcgaggattatgaagatgttgaagttagaactaattataaagttatctctgagaaatttggacaggattctcaggaagttaagaatttcaagaaggctgtcgctgtgatttctagagatcatgcaagaacaccgtatccatggagttcaaagggaccaaatgctggatttaatgactccgctaagccatggttcaaggtcaatgaggcctacaaggatggtatcaatgaggaagatgaggaaaatgatccaaattccgttttgaacttctggaaaaaggccattaaagtcaggaaggaaaacaagcatatcatggtttacggttacaatttcgaattctatgagcttgacaataataagatctttgctttcacaaaggaatatgaaaacgagaaactctttgccatctttaatttcaccaaagaggatgtttccttcaaattcccgggtaatgaaaagtacgttttgttctttggtactcatgataatgttgatggttcaaagacagttttgaaaccatgggaaggccgtctctaccacgttgagtag

SEQ ID NO: 12 - protein, 586 aa, Dekkera anomalus
MTSELAKHPETKPQWWKEATFYQIYPASFKDSNNDGWGDINGIRSKLSYLKSLGITAVWISPFYDSPQKDMGYDIRDYDKVWPTYGTNEEIFHLIKEAHKLDIRIVVDLVINHCSDEHKWFEESRSSLDNPKRDWFYWRKPKGYTKDGKPIPPNNWRSFFGGSAWEYDERTKEFYLHLFAVGQPDLNWENPVCRSAIFEDSMGFWLRNGVDGFRIDTAFLYSKDTTFPDAPITNPKDDFQDPGNFVSNGPRIHEFHKLMHHFMLSQIDDKRTIMTVGEVGHAGNDARVAYTSSASHELSEIFTFKHTDAGTSEKFRYDLVPFTLKDWKLAVADSFRWIDGTDSWSTIYFENHDQARSVSRFTDDSPESRVPAAKMLASLQICMTGTMYVYQGQELGCINLKNTPLEDYEDVEVRTNYKVISEKFGQDSQEVKNFKKAVAVISRDHARTPYPWSSKGPNAGFNDSAKPWFKVNEAYKDGINEEDEENDPNSVLNFWKKAIKVRKENKHIMVYGYNFEFYELDNNKIFAFTKEYENEKLFAIFNFTKEDVSFKFPGNEKYVLFFGTHDNVDGSKTVLKPWEGRLYHVE

SEQ ID NO: 13 - DNA, 1770 bp, Dekkera anomalus
atgaataaaattgaagataatgacagctctgcttcagagttggacattcctaaggcagataacgagcacatcgaaaactctgctccaaatgatgtcgatgatgagatcatctctcgtttccttggtgcttctgatgaagtgaaggaaggtgagaagcaagaaaaggagatgggcttcaaggctgccatgaaagcatatccaaagtgtgctgcatggtctgttgtcttatccagtggacttatcatggaaggttacgatacctgtttgttgagttctatgtatgctctcccaatcttctgtgaacgttttggtacatacgatacgagctcgaaggtttgggaaattactgcaaggtggcagattggtttaagtatgtgtgttaactgtggtgaaatcattggtttgcagatgtctggttggtttgcagacagatttggttatagatataccttaattgcttctatgggcttgcttgccgcatttacatttattattttctttgctaaggatattgctatgattgctgttggtcagattctatgtggtattccatggggattcttccagacgttggctgtttcttatgcttcggaggtttgtccgttagctttgagatactaccttaccagttatgtgaatgtttgctgggtgtttggacagttgtttgcatcaggtatcatgaagaactctcaggaaagaatggaacattctaatatgggatggagacttccatttggtcttatgtggatctggcctgttcctattgctcttggaatcttcttagcaccggaatctccatggtggctcgttaaaaagaataggtttgcagaggcagcaaacagtttgagaagacttctttctatcaaggatcacgatcagaaagagatggttgttgatacgatggtcaagagaatgaagtttactggtgataaggaagctagtaagggtgaagagattggttcatactgggaatgcttccacaagaagcactggagaagaactagaatcgctagtatggcttggattggacaaaatcttactggttcgacattgatgggatactcgacatacttttatgaaaaggcaggattatctacgtcatactcatttgacttcagtattattcaatactgtcttggtattgtcggtgctgtgatctcatggattttatctaactctgttggacgtgccacaatttacattggtggtatgggtgttcaaactataattttgtgggtcacaggtattctcggttttgttcatcaaagctctgctgttggttgggctgttggatcgttgttgttggtcttcactttggtctacgacagcactgtcggaccagttacctactgtattgttcctgagattccatctggtaaggttagaactaagactgttgtcattgctagaaatctttacaacattgccggtatctttaatgctgttattactccataccagttgaactctacacagtggaactggggtgccaagagtggtattttctgggcttgctttgccacgttcttcttggcatggtccatttgggatcttccggagaccaagggcagaacctttgcagatattgaccagttgttcgaagaacatattccagcaagaaagttcaagagcactgtcgttgatccattcaataacgccaagatgcttcagcagttcgacaagaataaacttaacgagcttgcaaatattaaggctgagacagcaatgaatgataatggttccgatattgagaagactgctgataactaa

SEQ ID NO: 14 - protein, 589 aa, Dekkera anomalus
MNKIEDNDSSASELDIPKADNEHIENSAPNDVDDEIISRFLGASDEVKEGEKQEKEMGFKAAMKAYPKCAAWSVVLSSGLIMEGYDTCLLSSMYALPIFCERFGTYDTSSKVWEITARWQIGLSMCVNCGEIIGLQMSGWFADRFGYRYTLIASMGLLAAFTFIIFFAKDIAMIAVGQILCGIPWGFFQTLAVSYASEVCPLALRYYLTSYVNVCWVFGQLFASGIMKNSQERMEHSNMGWRLPFGLMWIWPVPIALGIFLAPESPWWLVKKNRFAEAANSLRRLLSIKDHDQKEMVVDTMVKRMKFTGDKEASKGEEIGSYWECFHKKHWRRTRIASMAWIGQNLTGSTLMGYSTYFYEKAGLSTSYSFDFSIIQYCLGIVGAVISWILSNSVGRATIYIGGMGVQTIILWVTGILGFVHQSSAVGWAVGSLLLVFTLVYDSTVGPVTYCIVPEIPSGKVRTKTVVIARNLYNIAGIFNAVITPYQLNSTQWNWGAKSGIFWACFATFFLAWSIWDLPETKGRTFADIDQLFEEHIPARKFKSTVVDPFNNAKMLQQFDKNKLNELANIKAETAMNDNGSDIEKTADN

SEQ ID NO: 15 - DNA, 1767 bp, Dekkera bruxellensis
atgagtatgattgaagacaataatagttccgcctcccaacttgatggggcaaaggcggacaaagaacatatcgaaaattcagcaccaaatgatgttgatgacgaaattattactcgtttccttggtgcttctgatgaggtgaaggaaggtgagaaaaatgagaaagaaatgccttttaaagaagctatcagagcttatcctaaatgtgcactttggtctgttatcctttctagtggacttattatggagggttacgatacctgcttgttgaactctatgtatgctcttccaattttctgcgaacggtttggaacttatgacacaggttctaaggtttgggagattagtgccaaatggcaaattggtttgagtatgtgtgttaactgtggtgagattgttggtttgcagatgtctggttggtttgcagacagatttggttatagatatactttgattgtgtctttattcttccttgccgcctttacctttatcatcttctttgcaagaaatattgcaatgatcgccgttggacaaattctctgtggtattccatggggattcttccaaacgttagccgtttcttatgcctctgaggtttgcccattagccttgagatattaccttaccagttatgtgaatgtctgctgggtgtttggtcagttgtttgcctctggtattatgaagaactctcaggaaagaatggaacacagtgatatgggatggagacttccatttggtctcatgtggatttggcctgttcctattgctatcggtattttcttggcaccagaatctccatggtggttggtgaagaagaatagactttcacaagcagcaaacagtttaagaagacttctttctatcaaggatcttgatcaaaaagaagttgttgttgatacaatggttaagagaatgaaatttactggtgacaaggaacaatctaaaggagatattggaacatactgggaatgcttcaacaagaagaactggaggagaactagaatcgccagtatggtctggattggacagaatcttactggttcgacattgatgggatattctacctatttctatgagaaagcaggattgtcaacctcttactcatttgacttcagtattattcagtattgtcttggtatcgttggtgcagttatttcatggattttgtcgaattctgttggccgtgcaacaatttacattagtggaatggccattcagaccgttcttttgtggatcacaggtattcttggtttcgtccatcagacaactgccattggatgggccgttggatctttattattgattttcaccttgatatacgacagtactgttggaccagttacttactgtattgtccctgaaattccatcgggtaaagttagaaccaagactgttgttatttcaagaaacctttacaacatcgcaggtattttcaatgctgttatcactccatatcagttgaattctacacagtggaactggggtgctaagagtggtattttctgggcttgctttgctacattctttttggcatggtctatctgggatcttcccgaaaccaagggcagaacatttgccgatatcgaccagttgtttgaggagcacgtttcagcaagaaagttcaagagtactgtcgttgatccatttaacaacgctaagatgattcaacaattcgacaagactaaacttgatgagcttggaaacattaaggccgaagatgaggctttagatagctccgacattgagaagagcggtgcttctaactga

SEQ ID NO: 16 - protein, 588 aa, Dekkera bruxellensis
MSMIEDNNSSASQLDGAKADKEHIENSAPNDVDDEIITRFLGASDEVKEGEKNEKEMPFKEAIRAYPKCALWSVILSSGLIMEGYDTCLLNSMYALPIFCERFGTYDTGSKVWEISAKWQIGLSMCVNCGEIVGLQMSGWFADRFGYRYTLIVSLFFLAAFTFIIFFARNIAMIAVGQILCGIPWGFFQTLAVSYASEVCPLALRYYLTSYVNVCWVFGQLFASGIMKNSQERMEHSDMGWRLPFGLMWIWPVPIAIGIFLAPESPWWLVKKNRLSQAANSLRRLLSIKDLDQKEVVVDTMVKRMKFTGDKEQSKGDIGTYWECFNKKNWRRTRIASMVWIGQNLTGSTLMGYSTYFYEKAGLSTSYSFDFSIIQYCLGIVGAVISWILSNSVGRATIYISGMAIQTVLLWITGILGFVHQTTAIGWAVGSLLLIFTLIYDSTVGPVTYCIVPEIPSGKVRTKTVVISRNLYNIAGIFNAVITPYQLNSTQWNWGAKSGIFWACFATFFLAWSIWDLPETKGRTFADIDQLFEEHVSARKFKSTVVDPFNNAKMIQQFDKTKLDELGNIKAEDEALDSSDIEKSGASN

SEQ ID NO: 17 - DNA, 1761 bp, Dekkera bruxellensis
atggctagtgagttatcaaagcatccagagacaaccccagaatggtggaaggaagctacgttctatcagatttatccagcttctttcaaggactcgaataatgatggctggggtgatattaatggtatcaggtcgaagctttcctatttaaaagacttaggaattactgcagtctggatctgtccattctatgattcaccccaacaggatatgggctatgatatcagagattatgacaaagtctggccaacatatggtaccaatgaagatattttcaatctcataaaggaagctcatgatttaggtatcaagattttggtggacttggtcattaatcactgctcatctgagcataaatggttcaaggaaagtagagccagtaagaataacgcaaaaagagattggttctactggagaaagccaaagggatttaaaaaggacggcaccccacttcctccaaacaattggagatcatattttggaggttcggcctggcaatacgacgagaaaacaggtgagttctatttacgtatgtttgcttctggtcagccagatttgaactgggaaaatttggattgtagggctgcaatctttgaagattctatgggattttggttaagaaatggtgttgatggctttagaattgacactgcctatctctactcgaaggacaccaccttcccagatgctcctgtcaccaaccctaaaagtgaatatcaagatccgggtgcctttgttgcaaatggaccaaggattcatgaattccataagttgatgcactattacatgctttcgcagattaatgataaaagaataatcatgactgttggtgaagttggaactgctggtaatgacgttaggttacgttatactagtgccaagtcaaatgaagtatcagagatatttactttcaagcatactgagcttggtgtttcaaaagattttaagtttgatcttgttccatttactttaaaggagtggaaacttgctttggctgattcttttctttggatcaatggcacagattcatggtctacagtttatttggagaatcatgaccaggctagatcaatttcaagatttgccgatgactccactcctgagtcccgtgtggcttctggaaagatgttggctgctctccttgtgagtttgactggaactatgtacatttatcagggccaggaattgggatctattaatttgaagaatactcctattgaagagtatgaggacgtggaagtgaaaactaattacaaaattgtttcagagaaatatggagccgattctaaggaaatgaaagattatataaagggggttagctctgtttcaagggatcatgctaggacaccattaccatggagctctaagggatccactggaggattcagtgattccgcaaagccatggtttataatgaataaaacattcaaggacggtatcaatgttgcagatgaaaccaaggatccaaactcggttcttaacttctggaagagagcaattaaagttagaaaggagaataaagatattcttgtttacggttataacttcaaattttatgatcttgaaagcaacgaaatttttgcgtttacaaaagaatatgataccaagaggttgtttgcaatatttaacttcaccaaaaaggatgtttccttcaagtttcctgatgcaaagagctacaagctattctttggaacacatccagatgcaaatggttctgttacaagcttgaaaccttgggaaggtcgtctttattatgcaaat

SEQ ID NO: 18 - protein, 381 aa, Dekkera bruxellensis
MASELSKHPETTPEWWKEATFYQIYPASFKDSNNDGWGDINGIRSKLSYLKDLGITAVWICPFYDSPQQDMGYDIRDYDKVWPTYGTNEDIFNLIKEAHDLGIKILVDLVINHCSSEHKWFKESRASKNNAKRDWFYWRKPKGFKKDGTPLPPNNWRSYFGGSAWQYDEKTGEFYLRMFASGQPDLNWENLDCRAAIFEDSMGFWLRNGVDGFRIDTAYLYSRDTTFPDAPVTNPKSEYQDPGAFVANGPRIHEFHKLMHYYMLSQINDKRIIMTVGEVGTAGNDVRLRYTSAKSNEVSEIFTFKHTELGVSKDFKFDLVPFTLKEWKLALADSFLWINGTDSWSTVYLEIMTRLDQFQDLPMTPLLSPVWLLERCWLLSL

SEQ ID NO: 19 - DNA, 1770 bp, Dekkera bruxellensis
atgaataaaatcgaagataacgatagttctgcttcggagctggatgttcccaaagcagataatgaacatatcgaaaatgccgcaccaaatgatgtcgatgacgagatcattactcgtttccttggtgcttctgatgaagtgaaggaaggtgaaaaacaagagaaagagatgggattcaaggctgccatcaaagcatatcccaaatgtgcaatctggtctgttatcctttctagtggacttattatggagggttacgatacctgcttgttgaactctatgtatgctcttccaattttctgcgaacggtttggaacttatgacacaggttctaaggtttgggagattagtgccaaatggcaaattggtttgagtatgtgtgttaactgtggtgagattgttggtttgcagatgtctggttggtttgcagacaagtttggttatagatatactttgattggttccatgtttttccttgccgcctttacctttatcatcttctttgcaagaaatattgcaatgatcgccgttggacagattctttgtggtattccatggggattcttccaaacgttagccgtttcttatgcctctgaggtttgccccttagctttgagatattaccttaccagttatgtgaatgtctgctgggtgtttggtcagttgtttgcctctggtattatgaagaactcccaggaaagaatggaacacagtgatatgggatggagacttccatttggtctcatgtggatctggcctgttcctatttctatcggtattttcttggcaccagaatctccatggtggttggtgaagaagaatagactttcacaagcagcaaacagtttaagaagacttctttctatcaaggatcttgatcaaaaagaagttgttgttgatacaatggttaagagaatgaaatttactggtgataaggaacaggctaaaggagatattggaacgtactgggaatgcttcaacaagaagaactggaggagaaccagaatcgccagtatggtctggattggacagaatcttactggttcgacattgatgggatattctacctacttctatgagaaagcaggattgtcaacctctttctcatttgacttcagtattattcagtattgtcttggtatcgttggtgcagttctttcatggattatgtctaattcgatgggccgtgcaacaatttacattggtggaatggccattcagaccgttcttttgtggatcacaggtattcttggtttcgtccatcagacaactgccattggatgggccgttggatctttattattgatttttaccttgatatacgacagtactgttggaccagttacttactgtattgtccctgaaattccatcgggtaaagttagaaccaagactgttgttattgcaagaaacctttacaacatcgcaggtattttcaatgctgttatcactccataccagttaaattctacacagtgggactggggtgctaagagtggtattttctgggcttgctttgctacattctttttggcatggtctatctgggatcttcccgaaaccaagggcagaacatttgccgatatcgaccagttgtttgaggagcacgtttcagcaagaaagttcaagagtactgtcgttgatccatttaacaatgctaagatgcttcagcaatttgacaagactaaacttaatgagcttgccaatattaaggctgaaaaccaaatggccgaaaatgattcagatattgagaaggccagtgtggctaattaa

SEQ ID NO: 20 - protein, 589 aa, Dekkera bruxellensis
MNKIEDNDSSASELDVPKADNEHIENAAPNDVDDEIITRFLGASDEVKEGEKQEKEMGFKAAIKAYPKCAIWSVILSSGLIMEGYDTCLLNSMYALPIFCERFGTYDTGSKVWEISAKWQIGLSMCVNCGEIVGLQMSGWFADKFGYRYTLIGSMFFLAAFTFIIFFARNIAMIAVGQILCGIPWGFFQTLAVSYASEVCPLALRYYLTSYVNVCWVFGQLFASGIMKNSQERMEHSDMGWRLPFGLMWIWPVPISIGIFLAPESPWWLVKKNRLSQAANSLRRLLSIKDLDQKEVVVDTMVKRMKFTGDKEQAKGDIGTYWECFNKKNWRRTRIASMVWIGQNLTGSTLMGYSTYFYEKAGLSTSFSFDFSIIQYCLGIVGAVLSWIMSNSMGRATIYIGGMAIQTVLLWITGILGFVHQTTAIGWAVGSLLLIFTLIYDSTVGPVTYCIVPEIPSGKVRTKTVVIARNLYNIAGIFNAVITPYQLNSTQWDWGAKSGIFWACFATFFLAWSIWDLPETKGRTFADIDQLFEEHVSARKFKSTVVDPFNNAKMLQQFDKTKLNELANIKAENQMAENDSDIEKASVAN

SEQ ID NO: 21 - DNA, 1761 bp, Dekkera bruxellensis
atgtcatcagaattagcaaagcaccctgaaacaaaaccacaatggtggaaagaggccattttctatcagatttacccggcctccttcaaggattcgaataacgatggatggggagatattaacggtatcagatccaaactctcttacttgaagagtcttggcattacagcagtctggatctctccattctacgactctcctcaaaaggatatgggatatgatattagagattatgacaaggtctggccaacatatggaacaaatgacgaaattttccagttgatcaacgaggctcacaaacttgatattaagattgtggttgatttggttatcaatcattgctcagacgaacacaaatggtttgaagagtccagggcaagcctcaacaatcctaagagagattggttttactggaggaagccaaagggctacacgaaggaaggcaaaccaatcgctccaaataactggagatcatgctttggtggatctgcatggcaatatgatgaaagaaccaaggagttttatttgcatctatttgcagttggacagccagacctaaactgggagaatcctgtttgcagggctgccgtgtttgaagattccatgggattctggttaagaaatggtgttgatggcttcagaatcgatactgcctttctttattcaaaggacaccacattccctgatgctcctgttaccaatccaaaggatgacttccaggatccgggcaacctcgtttcaaatggtcctagaattcacgagttccataagttgatgaaccacttcatgctttctcagattgatgataaaagaacgatcatgaccgtcggagaagttggtcatgcaggtaatgatgcaaggttatcgtataccagtgctaaatcccacgaattatccgagatctttacgtttaagcacacggacattggaacgtcggagaaattcagatataacttggctccattcactttgaaggactggaaattggctgtcgccgactctttccgctggattaatggtaccgattcatggtcgaccatatatttcgaaaaccacgatcaggctaggtctgtttctagatttactgatgattcgccagagtctcgtgttcctgcggctaagatgcttgcttcccttcaaatctgtatgacaggtactatgtatgtctatcagggtcaggaattgggctgcatcaacctgaagaataccccacttaaggattatgaagatgtcgaagttagaacaaactataaggttatttcagaaaagtttggaaaagactctcaggaagtcaaggaattcgcgaaggctgtggccgtcatgtcgagagatcatgcaagaactccatatccatggagttctaagggaccaaatgctggatttaatgactctgccaagccatggtttaaggtcaacgaggcatataaagacggtattaatgaggaggatgaagagaaagatccaaaatctgttcttaatttctggagaaaggctattaaaactagaaaggaaaataagaatattctggtttatggttacaactttgagttctatgagcttgacaataacaaaatctttgctttcaccaaggagtatgatgatcagaagctttttgctattttcaatttcactaaagaagatgtttccttccagttcccagattccaagaaatacactttgttcttcggtacgcatgataatgccgatggctctgtcacaaccttgaaaccatgggaaggccgcctttaccacgttgagtaa

SEQ ID NO: 22 - protein, 586 aa, Dekkera bruxellensis
MSSELAKHPETKPQWWKEAIFYQIYPASFKDSNNDGWGDINGIRSKLSYLKSLGITAVWISPFYDSPQKDMGYDIRDYDKVWPTYGTNDEIFQLINEAHKLDIKIVVDLVINHCSDEHKWFEESRASLNNPKRDWFYWRKPKGYTKEGKPIAPNNWRSCFGGSAWQYDERTKEFYLHLFAVGQPDLNWENPVCRAAVFEDSMGFWLRNGVDGFRIDTAFLYSKDTTFPDAPVTNPKDDFQDPGNLVSNGPRIHEFHKLMNHFMLSQIDDKRTIMTVGEVGHAGNDARLSYTSAKSHELSEIFTFKHTDIGTSEKFRYNLAPFTLKDWKLAVADSFRWINGTDSWSTIYFENHDQARSVSRFTDDSPESRVPAAKMLASLQICMTGTMYVYQGQELGCINLKNTPLKDYEDVEVRTNYKVISEKFGKDSQEVKEFAKAVAVMSRDHARTPYPWSSKGPNAGFNDSAKPWFKVNEAYKDGINEEDEEKDPKSVLNFWRKAIKTRKENKNILVYGYNFEFYELDNNKIFAFTKEYDDQKLFAIFNFTKEDVSFQFPDSKKYTLFFGTHDNADGSVTTLKPWEGRLYHVE

SEQ ID NO: 23 - DNA, 666 bp, Dekkera bruxellensis
atgtatacgaatgttctaatatttatatcgaacaatttgcaattagttcgcctgtgcatttcatcaaataattgccattgctgcataaccaccacattttttgagttgaaaaataacaagccttatattcagaaaatgaaactcccttgctatcaaaacaatacgccccttgatccttccttcgatgatgacctgaaggacgttcatctcgtctatgattatgatgccacagattcgaacggaagaccagaaaaatggaggtatgaaatatggtttttctcagaaaataaaattgtttatgcgattcatggtggtccaatggcaggaaggattaattatcaaacggttgcttatcaatgtgtacgccctggagaaatatggcagataaattggcttgaagaaacaggcactgttgtgtcaatagtttatgacattgtgaataaaacggtaaacggacttctatgcttttctaagggacattgggaaaattctgaagctgctcatggggataaaagaaacccagatgactttgcccgttggagaaatttggccaagcagggcattcaaaccgatcgtttcgtcttggttgaaagagcccatatattgaaatcatttaaaggtcagggtgatttggaaccgatcgaacctgatgcaattaccttttag

SEQ ID NO: 24 - protein, 221 aa, Dekkera bruxellensis
MYTNVLIFISNNLQLVRLCISSNNCHCCITTTFFELKNNKPYIQKMKLPCYQNNTPLDPSFDDDLKDVHLVYDYDATDSNGRPEKWRYEIWFFSENKIVYAIHGGPMAGRINYQTVAYQCVRPGEIWQINWLEETGTVVSIVYDIVNKTVNGLLCFSKGHWENSEAAHGDKRNPDDFARWRNLAKQGIQTDRFVLVERAHILKSFKGQGDLEPIEPDAITF

SEQ ID NO: 25 - DNA, 1776 bp, Dekkera bruxellensis
atgagtaagattgaagataaaaatagctcgaattcgcagcttgagctaccaaaagcagataacgagcatatcgaaaacgccgcaccaaatgatgtcgatgacgagatcatcactcgtttccttggtgcttctgatgaggtgaaggaaggtgaaaaacaagagaaagagatgggattcaaggctgccatcaaagcatatcccaaatgtgcggtttggtcagttgttctttctactggacttattatggagggttacgatacctgcttgttgaactctatgtatgctcttccaattttctgcgaacggtttggaacttatgacacaggttctaaggtttgggagattagtgccaaatggcaaattggtttgagtatgtgtgttaactgtggtgagattattggtttgcagatgtctggttggtttgcagacaagtttggttatagatatactttgattggttccatgtttttccttgccgcctttaccttcatcatcttctttgcaagaaatattgcaatgatcgccgttggacagattctctgtggtattccatggggattcttccaaacgttagccgtttcttatgcctctgaggtttgcccattagctttgagatattaccttaccagttatgtgaatgtctgctgggtgttcggtcagttgtttgcctctggtattatgaagaattctcaggaaagattggagcacagtaatatgggatggagacttccatttggtctcatgtggatttggcctgttcctatttctattggtattttcttggcaccagaatctccatggtggttggtgaagaagaatagatttacacaggcagcaaacagtttaagaagacttctttctatcaaggatcctaatcaaaaggaagttgttgtcgatacaatgatcaaaagaatgaagtttactggtgataaggaacaggctaaaggagatattggaacatactgggaatgcttcaacaagaagaactggaggagaaccagaatcgccagtatggtctggattgggcagagtgtgactggttcgacattgatgggatattctacttacttctatgagaaggcaggattgtctacctctttctcatttgacttcagtattattatgtactgccttggtattgctggtgcggtcatttcatggattttgtcgaattctgttggacgtgcatcgatttatatttggggtatgcttggacaagcaattctcttatgggttgttggaattcttggctttgtccatcagagttctgcaataggatgggctgttggttctcttttattggcttacactgttgtttatgatagcactgtcggacctgttacttactgtattgtcacagaaattccatcgggtaaagtcagaaccaagactgttgttattgcaagaaacctttataatattggtggtattttcaatgctgttatcactccataccagttgaattctacacagtggaactggggtgctaagagtggtattttctgggcagtttctgcaacactatggttagcatggtctatctgggatcttccagagaccaaaggcagaacatttgccgatatcgaccaattgttcgaggagcatatctcagcaagaaagttcaagagtactgatgttgatccattcaacaatgctaagatgcttcgggaatttgacaaggataagcttaatgagcttggtaatcttaataatgaaagttctgattatgataataagtctgatattgaaaaggctgaagctacaccagttaactag

SEQ ID NO: 26 - protein, 591 aa, Dekkera bruxellensis
MSKIEDKNSSNSQLELPKADNEHIENAAPNDVDDEIITRFLGASDEVKEGEKQEKEMGFKAAIKAYPKCAVWSVVLSTGLIMEGYDTCLLNSMYALPIFCERFGTYDTGSKVWEISAKWQIGLSMCVNCGEIIGLQMSGWFADKFGYRYTLIGSMFFLAAFTFIIFFARNIAMIAVGQILCGIPWGFFQTLAVSYASEVCPLALRYYLTSYVNVCWVFGQLFASGIMKNSQERLEHSNMGWRLPFGLMWIWPVPISIGIFLAPESPWWLVKKNRFTQAANSLRRLLSIKDPNQKEVVVDTMIKRMKFTGDKEQAKGDIGTYWECFNKKNWRRTRIASMVWIGQSVTGSTLMGYSTYFYEKAGLSTSFSFDFSIIMYCLGIAGAVISWILSNSVGRASIYIWGMLGQAILLWVVGILGFVHQSSAIGWAVGSLLLAYTVVYDSTVGPVTYCIVTEIPSGKVRTKTVVIARNLYNIGGIFNAVITPYQLNSTQWNWGAKSGIFWAVSATLWLAWSIWDLPETKGRTFADIDQLFEEHISARKFKSTDVDPFNNAKMLREFDKDKLNELGNLNNESSDYDNKSDIEKAEATPVN

SEQ ID NO: 27 - DNA, 1764 bp, Dekkera bruxellensis
atgaataaaatcgaagataacgatagttctgcttcggagctggatgttcccaaagcagataatgaacatatcgaaaatgccgcaccaaatgatgtcgatgacgagatcatcactcgtttccttggtgcttctgatgaagtgaaggaaggtgaaaaacaagagaaagagatgggattcaaggctgccatcaaagcatatcccaaatgtgcaatttggtctgttatcctttctagtggacttatcatggagggttacgatacctgcttgttgaactctatgtatgctcttccaatcttctgtgaacggtttggaacttatgacacaggttctaaggtttgggagattagtgccaaatggcaaattggtttgagtatgtgtgttaactgtggtgagattattggtttgcagatgtctggttggtttgcagacaagtttggttatagatatactttgattggttccatgtttttccttgccgcctttaccttcatcatcttcttcgcaagaaatattgcaatgatcgccgttggacaaattctctgtggtattccatggggattcttccaaacgttagccgtttcttatgcctctgaggtttgcccattagccttgagatattaccttaccagttatgtgaatgtctgctgggtgtttggtcagttgtttgcctctggtattatgaagaactctcaggaaagaatggagcacagtgatatgggatggagacttccatttggtctcatgtggatctggcctgttcctattgctatcggtattttcttggcaccagaatctccatggtggttggtgaagaagaatagactttcacaagcagcaaacagtttaagaagacttctttctatcaaggatcctaatcaaaaggaaattgttgttgatacaatggttaagagaatgaagttcactggtgataaggaacaggctaaaggagatattggaacgtactgggaatgcttcaacaagaagaactggaggagaactagaattgccagtatggtctggattggacagaatcttactggttcgacattgatgggatattctacctacttctatgaaaaggcaggattgtcaacctcttactcatttgacttcagtattattcagtattgtcttggtattgttggtgcagttctttcatggattatgtccaattcgatgggccgtgcaacaatttacattagtggaatggccattcagaccgttcttttgtggatcacaggtattcttggtttcgtccatcagacaactgccattggatgggccgttggatctttattattgattttcaccttgatatacgacagtactgttggaccagttacttactgtattgtccctgaaattccatcgggtaaagttagaaccaagactgttgttattgcaagaaatctttacaacatcgcaggtattttcaatgctgttatcactccataccagttgaattctacacagtgggactggggtgctaagagtggtattttctgggcttgctttgctacattctttttggcatggtcaatctgggatcttcccgaaaccaagggcagaacatttgccgatatcgaccaattgtttgaggagcacgtttcagcaagaaagttcaagagtactgatgttgatccatttaacaacgctaagatgattcagcaatttgacaagactaaacttaatgagcttgctaatattaaagctgaaggttctggagctgataacgataactcatccgatatcgaaaaggcatag

SEQ ID NO: 28 - protein, 587 aa, Dekkera bruxellensis
MNKIEDNDSSASELDVPKADNEHIENAAPNDVDDEIITRFLGASDEVKEGEKQEKEMGFKAAIKAYPKCAIWSVILSSGLIMEGYDTCLLNSMYALPIFCERFGTYDTGSKVWEISAKWQIGLSMCVNCGEIIGLQMSGWFADKFGYRYTLIGSMFFLAAFTFIIFFARNIAMIAVGQILCGIPWGFFQTLAVSYASEVCPLALRYYLTSYVNVCWVFGQLFASGIMKNSQERMEHSDMGWRLPFGLMWIWPVPIAIGIFLAPESPWWLVKKNRLSQAANSLRRLLSIKDPNQKEIVVDTMVKRMKFTGDKEQAKGDIGTYWECFNKKNWRRTRIASMVWIGQNLTGSTLMGYSTYFYEKAGLSTSYSFDFSIIQYCLGIVGAVLSWIMSNSMGRATIYISGMAIQTVLLWITGILGFVHQTTAIGWAVGSLLLIFTLIYDSTVGPVTYCIVPEIPSGKVRTKTVVIARNLYNIAGIFNAVITPYQLNSTQWDWGAKSGIFWACFATFFLAWSIWDLPETKGRTFADIDQLFEEHVSARKFKSTDVDPFNNAKMIQQFDKTKLNELANIKAEGSGADNDNSSDIEKA

SEQ ID NO: 29 - DNA, 1593 bp, Dekkera bruxellensis
atgggattcaagaaggctttctctacatatcctaaatcagctatctggtctgttgtcatctctagtggtattattatggagggatatgatacatgcttatcaatgatcgctcttccgatattttgtgagcggtttggtgtttggaatcctaagtcacaagtgtgggaaattccagcaaaatggcaatctgcaatgggaatgtgtgccaacgttggagaaattataggtttgcaaattgctggtactgttgcagacaggttcggttatcgttacactttaattggttcattattttctctagctgtttttatattcttgatcttctttgcaaagtcacttggtatgatctgtgctggtcaaattctttgtggaattccttggggattcttccagactttggctgtttcctatgcttctgaagtttgtcctatgacaatgagattctatcttaccagttatgttaacgtttgttggcttcttggccagttgtttggatctggtatcatgaagaattcacaagataatttagcacactctgatatgggctggaggcttccttacggactcatgtggatttggccagttcctattgcaatagctattttctgtgcaccagagtcaccttggtggcttgttaagaaaaacagagtttctaaggcagctcatagtcttagcagacttcttactatcaaggatccggatcaaaaagaaatagtcattgataccatgttaaagagaatgaaattcactagtgataaagaggaagccgacggggatattggatcctatcgggaatgtttcagcaaaaaacatattagaagaaccagaattgcaagtatggtttggattggacaaatgcttacaggctgtggtttgatgggttcatcgacatatttctacgaaaaagctggtctctcaacatcatactcgtttgatttcagtatcattcagtactgtatcggtattattggtgctattgtttcatggtttgtgtcaaacaatcttggtcgtgccaccctttatatctggggcacattctgccaaggtgtcttattatggatcatgggaatattaggatttgttaaacagactaatgctgtccgttggacatgtggttctatattgttggttcttagtttcgtttatgattgtactgttggacccgttacgtactgtatcgttacagaaattccttcaggaagagtgagaaccaagactgttatcatcgcaagaaatctttacaatctctttggtatctttgatgcattcatggtgccttaccaattaaactcaacacagtggaactggggcgcaaaatctggcttattctgggctgcatttgcaaccatttggtttgtctggggacttttcgatttaccagagaccaaaggcagaacatttgccgatattgatcagttattcgaggagaaagtttctgcaaggaagttcaagagcactgtcgtcgatccattcaacaacgctaaaatgcttcagcaattcgatagggagaaacttaacgaacttgccaatatccgtgccgatcgtgatattgaagatagtaaggttgatattgagaaagaaggcgataaataa

SEQ ID NO: 30 - protein, 530 aa, Dekkera bruxellensis
MGFKKAFSTYPKSAIWSVVISSGIIMEGYDTCLSMIALPIFCERFGVWNPKSQVWEIPAKWQSAMGMCANVGEIIGLQIAGTVADRFGYRYTLIGSLFSLAVFIFLIFFAKSLGMICAGQILCGIPWGFFQTLAVSYASEVCPMTMRFYLTSYVNVCWLLGQLFGSGIMKNSQDNLAHSDMGWRLPYGLMWIWPVPIAIAIFCAPESPWWLVKKNRVSKAAHSLSRLLTIKDPDQKEIVIDTMLKRMKFTSDKEEADGDIGSYRECFSKKHIRRTRIASMVWIGQMLTGCGLMGSSTYFYEKAGLSTSYSFDFSIIQYCIGIIGAIVSWFVSNNLGRATLYIWGTFCQGVLLWIMGILGFVKQTNAVRWTCGSILLVLSFVYDCTVGPVTYCIVTEIPSGRVRTKTVIIARNLYNLFGIFDAFMVPYQLNSTQWNWGAKSGLFWAAFATIWFVWGLFDLPETKGRTFADIDQLFEEKVSARKFKSTVVDPFNNAKMLQQFDREKLNELANIRADRDIEDSKVDIEKEGDK

SEQ ID NO: 31 - DNA, 1350 bp, Dekkera bruxellensis
atgcccgcgtttgtatcaaaatttggcgataaattggattctacaactccagaattttatcccataacgaaaggtatattactcggggtcggaagcctcggcgggtgcataggaggacttttaacaaaagccatggttccggtttttggtccccgaacaacattatttctatcaggtctaacatcaagcttaggcgatattattattttgctttcaaaaaactggttgcaagttgccgtcggacgagcagtttatggtataggctgtgggattatatgtgtcgtctgtcccatgctaattagtgatttatcaccaacgaataagcggggtctatttatctcaataatacagttgcatgttactattggtattattattggtgcacttacaatgtttataagtgatatacaatttagttcaaatgtgtccagccattggcaatacagctacccagtattgcagggtatattatttggaattatgacatgtgctgtaattttctttattccagagtcaccaacatggcttctatcaaagggtagacaaaatttcgataaagccgcaaaatcgctagcaagatgttcccagcttgctgtgtccagcactatagttcaggctcaaacagaaaatattgcagtactaatggcctcggttgaagatgatcaagatagtctgctagaaaagcttaacaagtcacttttaaggggccaaccaaaatacttgctaagaaccattacaggaatcgtactttcttgtcttcaacagttcactgggatcaattacttttttttttttagcgttgtcatattcaaaactgttggagtgaacaacccgtacattgtacctattttctttggttcggtcaatgttatcttttcccttctcagcgttttattaattgctcgatttcagagattaacgcttctaaagcttggatcattttttttggctcttacaatgtgtgtgtttacaactttaggtctcacattcagtgaaagtaacactggcgcaattatgatgataatggcatcgtgtatttttgttgccatttttgcaactacatggggcccattggtgagtataataatatctgagctttatcctcacagtatcaaagtcaaggcaatggctatatgtgggtgcgcaggctggatttcgacgtttctagtttcatttatgataccaatacttacaaaagtaataggctttgcattgggctatattttctgtgtcttaatcgtcttaggcatagcattcatatactattttgttccagaaacaagacatgttgaaaattcggcgttggatgcaatttatgaagctaatatttacccacgaaaattgttctga

SEQ ID NO: 32 - protein, 448 aa, Dekkera bruxellensis
PAFVSKFGDKLDSTTPEFYPITKGILLGVGSLGGCIGGLLTKAMVPVFGPRTTLFLSGLTSSLGDIIILLSKNWLQVAVGRAVYGIGCGIICVVCPMLISDLSPTNKRGLFISIIQLHVTIGIIIGALTMFISDIQFSSNVSSHWQYSYPVLQGILFGIMTCAVIFFIPESPTWLLSKGRQNFDKAAKSLARCSQLAVSSTIVQAQTENIAVLMASVEDDQDSLLEKLNKSLLRGQPKYLLRTITGIVLSCLQQFTGINYFFFFSVVIFKTVGVNNPYIVPIFFGSVNVIFSLLSVLLIARFQRLTLLKLGSFFLALTMCVFTTLGLTFSESNTGAIMMIMASCIFVAIFATTWGPLVSIIISELYPHSIKVKAMAICGCAGWISTFLVSFMIPILTKVIGFALGYIFCVLIVLGIAFIYYFVPETRHVENSALDAIYEANIYPRKLF

(19) United States
(12) Patent Application Publication
(10) Pub. No.: US 2023/0212487 A1
(43) Pub. Date: Jul. 6, 2023

(54) Yeast for Preparing Beverages Without Phenolic Off-Flavors

(71) Applicant: Carlsberg A/S, Copenhagen V (DK)

(72) Inventors: Marc Serra Colomer, Olot (ES); Zoran Gojkovic, Copenhagen V (DK); Jochen Förster, Copenhagen V (DK); Natalia Y. Solodovnikova, Copenhagen NV (DK); Ross Fennessy, Copenhagen V (DK)

(73) Assignee: Carlsberg A/S, Copenhagen V (DK)

(21) Appl. No.: 17/638,037

(22) PCT Filed: Aug. 28, 2020
(86) PCT No.: PCT/EP2020/074090

§ 371 Date: Feb. 24, 2022

(30) Foreign Application Priority Data
Aug. 30, 2019 (DK) PA 2019 70542
Aug. 30, 2019 (DK) PA 2019 70543

(51) Int. Cl. C12C 12/00; C12C 11/00; C12N 9/88; C12N 1/16

(52) U.S. Cl. CPC C12C 12/006; C12C 11/003; C12N 9/88; C12N 1/16

(57) ABSTRACT

The invention relates to yeast strains with useful characteristics, including not being capable of producing phenolic off-flavors and/or not capable of utilizing maltose or which has limited ability to utilize maltose. Also provided is methods of producing cereal based beverages without phenolic off-flavors and/or a low alcohol or a non-alcoholic malt and/or cereal based beverage, as well as beverages produced by these methods.

### TECHNICAL FIELD

The present invention relates to Dekkera yeast strains with reduced ability to convert p-coumaric acid into 4-ethylphenol and/or reduced ability to convert ferulic acid into 4-ethylguaiacol. The term Dekkera as used herein may refer both to teleomorph Dekkera strains as well as to anamorph Brettanomyces strains. The present invention further relates to Dekkera yeast strains, which are not capable of utilizing maltose or which has limited ability to utilize maltose. In addition, the invention relates to such yeast strains, which have both of the aforementioned properties. The present invention also provides methods of producing a malt and/or cereal based beverage comprising low levels of 4-ethylphenol and/or 4-ethylguaiacol, as well as beverages produced by these methods. Further provided are methods of producing a low alcohol or a non-alcoholic malt and/or cereal based beverage, as well as beverages produced by this method.

### BACKGROUND OF THE INVENTION

Dekkera yeast strains are sometimes used in the production of craft beer, due to their unique flavor profiles. However, in most beer styles Dekkera is typically viewed as a contaminant, because Dekkera normally produce several off-flavor, for example phenolic off-flavors.

Phenols represent a broad class of compounds that may be welcome or completely undesirable in beer or other beverages, depending on the brewer's intention and the target style. Phenolic flavors and aromas in beer are most often described as clovey, spicey, smokey, band-aid-like, or medicinal flavors and aromas. Thus, Dekkera is generally reported as a spoilage yeast responsible for off-flavor production in wine, beer, cider or dairy products leading to huge economic losses. In a few beer styles some of these flavors are considered appropriate.

Mukai et al., 2010, describes the production of phenolic off-flavors in Saccharomyces cerevisiae and the conversion of p-coumaric acid into 4-ethylphenol and ferulic acid into 4-ethylguaiacol. Mukai et al. identifies phenolic acids decarboxylase (PAD1) as being responsible for the conversion of p-coumaric acid into 4-vinyl-phenol that is further converted into 4-ethylphenol in Saccharomyces cerevisiae.

Harris et al., 2009 describes synthesis of volatile compounds using cell extracts from Dekkera and Brettanomyces species. Harris et al. describes a partial protein, which shares around 50-56% homology to the protein Pst2 of Candida and Saccharomyces. Pst2 in Dekkera has no described function. It is unlikely that Pst2 from Candida and Saccharomyces is involved in hydroxycinnamic acids catabolism. The partial protein has very limited sequence homology to the PAD enzyme of S. cerevisiae.

Alcoholic beverages are frequently prepared by fermentation of a carbohydrate rich liquid with yeast. For example, beer is prepared by fermenting wort with yeast. Wort contains a number of compounds, which can normally be utilized by yeast. For example wort is rich in sugars, in particular maltose as well as in amino acids and small peptides. Conventional yeast can utilize maltose and thus conventional yeast can ferment maltose to produce ethanol.

Alcohol-free beer and low-alcohol beer are beers with no alcohol or low alcohol content. These beers with a low alcohol content are often made by producing full-strength alcoholic beer and then removing the alcohol by a physical process, or simply by diluting the full-strength beers with water. Alternatively, alcohol-free beers can be made without fermentation. A drawback from these methods are often a lack of desirable flavors and/or presence of off-flavors compared to full-strength beer.

The use of non-conventional yeasts species has been explored more rigorously, since the choice of yeast can strongly influence the flavor profile of a beer. Dekkera species have been highlighted for beer flavoring, as their use result in features unachievable with conventional brewer's yeast, both in production of alcoholic beverages, as well as alcohol-free beer and low-alcohol beer.

The biochemical pathways involved in beer fermentation and aroma formation in brewer's yeasts have been extensively studied. However, very few studies have been done in Dekkera yeasts due to the complexity of its genome and the lack of genomic tools to perform gene deletions and transformations.

### SUMMARY OF THE INVENTION

Currently, beer produced by fermentation with a Dekkera yeast strain contains phenolic off-flavors. Thus, there are currently no methods of producing a malt and/or cereal based beverage comprising unique flavors produced by Dekkera yeast strains, but which at the same time contains no or little phenolic off-flavors. Interestingly, the invention provides Dekkera yeast strains, e.g. Dekkera bruxellensis and Dekkera anomalus (also known as Brettanomyces bruxellensis and Brettanomyces anomalus in their anamorph state), which are useful for the production of malt and/or cereal based beverages comprising low levels of 4-ethylphenol and/or low levels of 4-ethylguaiacol.

In particular, it is preferred that Dekkera yeast strains of the invention are not capable of converting more than 25% of p-coumaric acid into 4-ethylphenol when incubated in an aqueous solution comprising p-coumaric acid and/or not capable of converting more than 25% of ferulic into 4-ethylguaiacol, when incubated in an aqueous solution comprising ferulic acid. Hitherto the regulatory pathways involved in the production of phenolic off-flavors in Dekkera have been unclear. In brewer's yeast, the regulatory pathways involved in phenolic off-flavor production has been mapped, however, the Dekkera genome is significantly different to brewer's yeast.

Thus, the invention provides Dekkera yeast strains, which are not capable of converting more than 25% of p-coumaric acid into 4-ethylphenol and/or more than 25% of ferulic into 4-ethylguaiacol, hereby producing a beverage with reduced levels of 4-ethylphenol and/or 4-ethylguaiacol. The Dekkera yeast strains may in addition or alternatively not be able to able to utilize more than 2% maltose. The invention also provides novel methods of producing a beverage with a pleasant taste, by using Dekkera yeast strains, not capable of converting more than 25% of p-coumaric acid into 4-ethylphenol and/or more than 25% of ferulic into 4-ethylguaiacol.

In one aspect of the present invention is provided a method of producing a malt and/or cereal based beverage, said method comprising the steps of
i) providing an aqueous extract of malt and/or cereal kernels
ii) providing a Dekkera yeast strain, wherein said yeast strain is not capable of converting more than 25% of p-coumaric acid into 4-ethylphenol when incubated in an aqueous solution comprising p-coumaric acid
iii) fermenting said aqueous extract with said yeast strain
   thereby obtaining said malt and/or cereal based beverage.

Another aspect of the invention is to provide a Dekkera yeast strain, which is not capable of converting more than 25% of p-coumaric acid into 4-ethylphenol when incubated in an aqueous solution comprising p-coumaric acid. In one embodiment of the invention, said yeast strain is not capable of converting more than 25% of ferulic acid into 4-ethylguaiacol when incubated in an aqueous solution comprising ferulic acid.

Another aspect of the invention is to provide a malt and/or cereal based beverage comprising low levels of 4-ethylphenol, such as less than 0.5 mg/L, such as less than 0.3 mg/L, such as less than 0.1 mg/L 4-ethylphenol. In one embodiment of the invention, said malt and/or cereal based beverage comprises low levels of 4-ethylguaiacol, such as less than 1 mg/L of 4-ethylguaiacol, such as less than 0.8 mg/L, such as less than 0.6 mg/L, such as less than 0.5 mg/L of 4-ethylguaiacol.

Another aspect of the present invention is to produce a pleasant alcohol-free or low-alcohol beverage. Thus, one aspect of the present invention is produce a pleasant alcohol-free or low-alcohol beverage, having low levels of 4-ethylphenol and/or 4-ethylguaiacol.

The invention further provides Dekkera yeast strains, which are useful for the production of low-alcohol or alcohol-free beverages. In particular, the Dekkera yeast strains of the invention are not capable of utilizing maltose or has limited ability to utilize maltose, and accordingly, if added to an aqueous extract rich in maltose, said yeast produces only limited amounts of ethanol. This is in particular the case, if said aqueous extract contains only low levels of glucose. Hitherto the regulatory pathways involved in maltose metabolism in Dekkera have been unclear. In brewer's yeast, the regulatory pathways involved in maltose utilization are highly complex, however, the Dekkera genome is significantly different to brewer's yeast.

Thus, the invention further provides Dekkera yeast strains, which are not able to utilize more than 2% maltose, but which at the same time produces a full flavor low-alcohol or alcohol-free beer with a pleasant taste. The invention also provides novel methods of producing a low-alcohol or alcohol-free beverages with a pleasant taste, by using Dekkera yeast strains, which are not capable of utilizing more than 2% maltose.

### DESCRIPTION OF DRAWINGS

FIG. 1. Panel A) shows the content (mg/L) of p-coumaric acid, ferulic acid, 4-EP (4-ethylphenol) and 4-EG (4-ethylguaiacol) in beer fermented by CRL-2 and CRL-49 (both Dekkera bruxellensis) and CRL-90 (Dekkera anomalus). Fermentation was performed at 25° C. for 169 hours and the levels of p-coumaric acid, ferulic acid, 4-EP and 4-EG at the end of fermentation are shown. The results indicate that CRL-90 is not able to convert p-coumaric acid into 4-ethylphenol and had very reduced ability to convert ferulic acid into 4-ethylguaiacol. Panel B) shows the genomic set-up of CRL-90 aligned to a reference Dekkera anomalus yeast strain, CRL-49. From the genomic set-up it is evident that the first 1 to 53,715 bp of the scaffold of CRL-90 is missing.

FIG. 2. Panel A) shows metabolic activity as determined by NADH production of various Dekkera yeast strains in a defined YNB medium supplemented with amino acids. Strains are grown in triplicates, and standard deviation is showed by color shading. The y-axis shows purple colour in Omnilog units as measured using the Omnilog® Biolog system. NADH production is measured by reduction of tetrazolium dye to purple formazan. Thus, the quantification of the strain metabolic activity was based on adding tetrazolium dye that is reduced to purple formazan dependent on yeast strain NADH production as a measure of metabolic activity. Strain growth can be correlated to the metabolic activity, and thus be determined based on generation of purple color. The x-axis shows the time measured in hours. G: Glucose; M: Maltose. FIG. 2 shows that CRL-90 (D. anomalus) and CRL-2 (D. bruxellensis) are the only yeast strains tested, which were not able to grow when maltose is present as sole carbon source. Panel B) shows the genomic set-up of CRL-90 aligned to a reference Dekkera anomalus yeast strain, CRL-49. From the genomic set-up it is evident that the first 1 to 40,470 bp of the scaffold are missing.

FIG. 3: Panel A) shows the fermentation curve in beer wort of five different Dekkera yeast strains. The y-axis represents the cumulative pressure measured with the ANKOM system with psi units. The x-axis shows time measured in hours. From the figure, it is evident that CRL-1, CRL-19, CRL-49 and CRL-50 are capable of utilizing the majority of the fermentable sugars present in the wort, whereas CRL-2 is only capable of utilizing a minority of the fermentable sugars present in the wort. Panel B) shows the fermentation curve in beer wort of one Dekkera bruxellensis yeast strain, CRL-2, and one Dekkera anomalus yeast strain, CRL-90. The y-axis represents the cumulative pressure measured with the ANKOM system with psi units. The x-axis shows time measured in hours. Both yeast strains, CRL-2 and CRL-90 were only able to utilize a minority of the fermentable sugars.

FIG. 4: shows the comparison of the protein sequences of various putative maltose transporters found in a reference genome of D. bruxellensis (MTRA5, MTRA4, MTRA3, MTRA2, MTRA1). The upper part of the table displays sequence identity in %. The lower part of the table shows the number of amino acid changes between transporters.

FIG. 5: Panel A) shows a nucleotide alignment of the MTRA1 gene sequences for CRL-1 (4 copies found), CRL-50 (3 copies found), CRL-19 (1 copy found), CRL-2 (1 copy found with 97.5% homology). The alignment displays the N-terminal nucleotide sequence of the MTRA1 transporter. It can be concluded that the copy found in CRL-2 has a completely different N-terminal nucleotide sequence compared to CRL-1, CRL-19 and CRL-50. Panel B) shows the amino acid sequence of all MTRA1 copies found in CRL-1, CRL-2, CRL-19 and CRL-50, from this alignment it can also be concluded that the N-terminal amino acid sequence of MTRA1 in CRL-2 is different from the amino acid sequence of MTRA1 in CRL-1, CRL-19 and CRL-50.

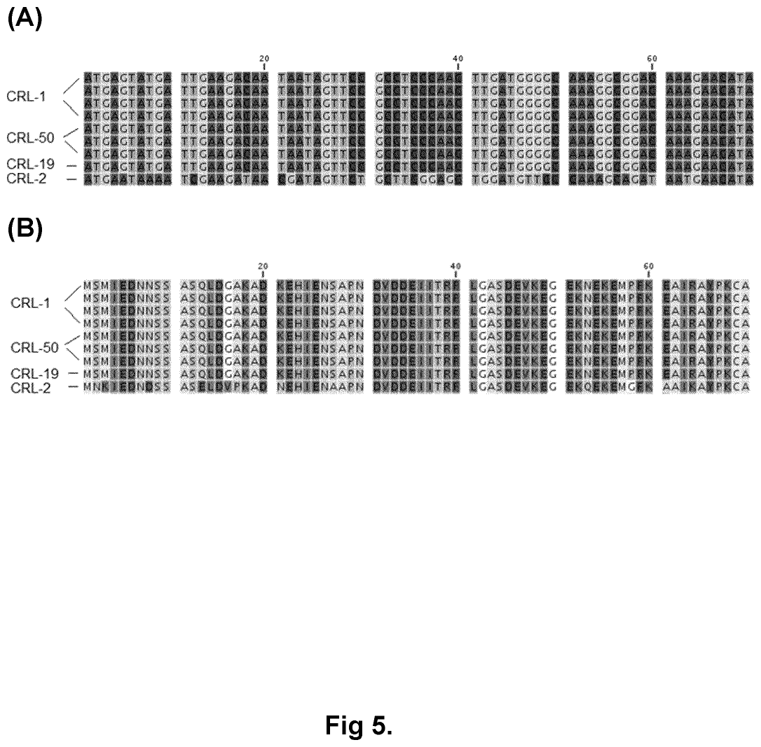

FIG. 6: Panel A) shows nucleotide alignment of a part of the ISOM(2) gene for CRL-1, CRL-19, CRL-50 and CRL-2. The arrow indicates the deletion at 1050 bp in ISOM(2) of CRL-2. Panel B) shows protein alignment of ISOM(2) of CRL-1 and CRL-2. It can be concluded that the deletion results in a frame shift, resulting in translation being truncated, and thus 50% of the ISOM(2) protein is not present in CRL-2.

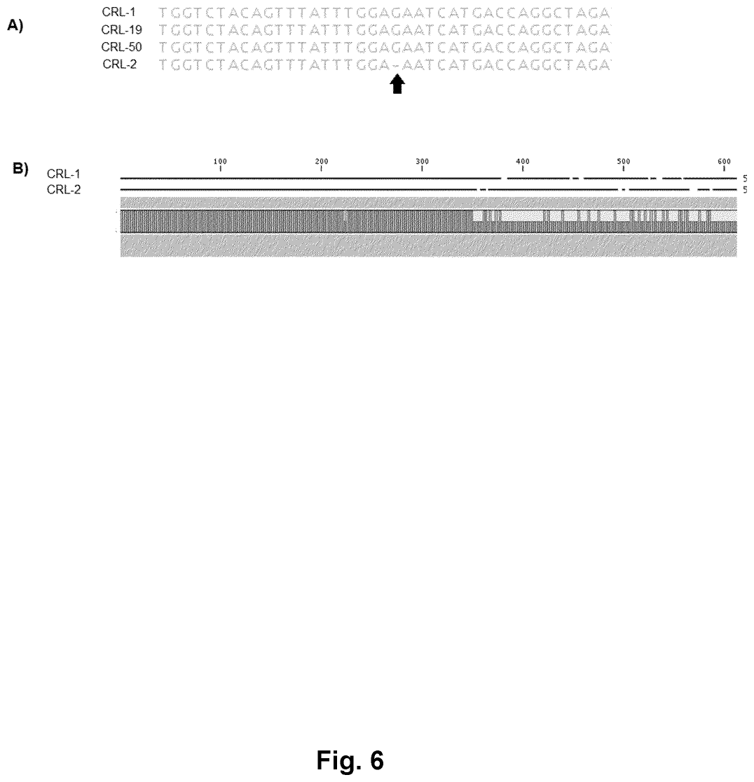

FIG. 7: shows 3D model structure of protein ISOM(2), produced with CLCGenomicsWorkbench 11. The part missing in the maltose negative Dekkera is colored in white.

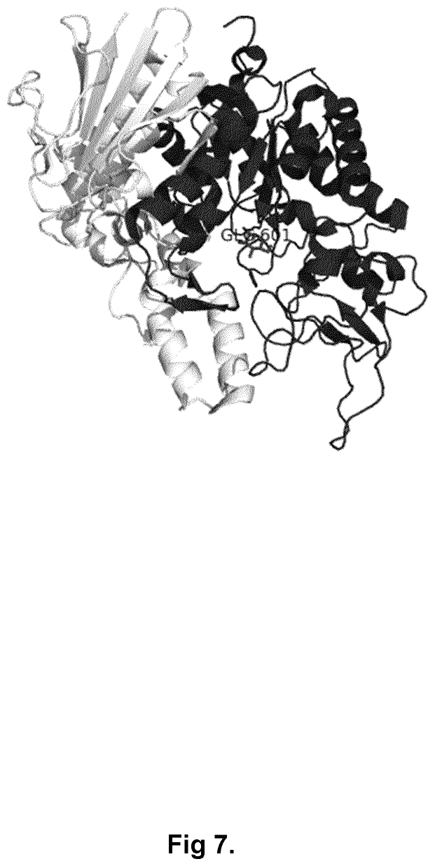

FIG. 8: shows the monoterpene alcohols measured in beers after fermentation with Dekkera applied as primary (A) or secondary (B) yeast strain. The sum of total monoterpene alcohols is indicated in μg/L below the strain name. CRL-1, CRL-2, CRL-19 and CRL-50 are Dekkera bruxellensis yeast strains, and CRL-49 is a Dekkera anomalus yeast strain.

FIG. 9: shows the genomic set-up of CRL-90 aligned to a reference Dekkera anomalus yeast strain, CRL-49. From the genomic set-up it is evident that the first 1 to 40,470 bp of the scaffold wherein ISOM(1), MTRA1 and MTRA2 are located is missing.

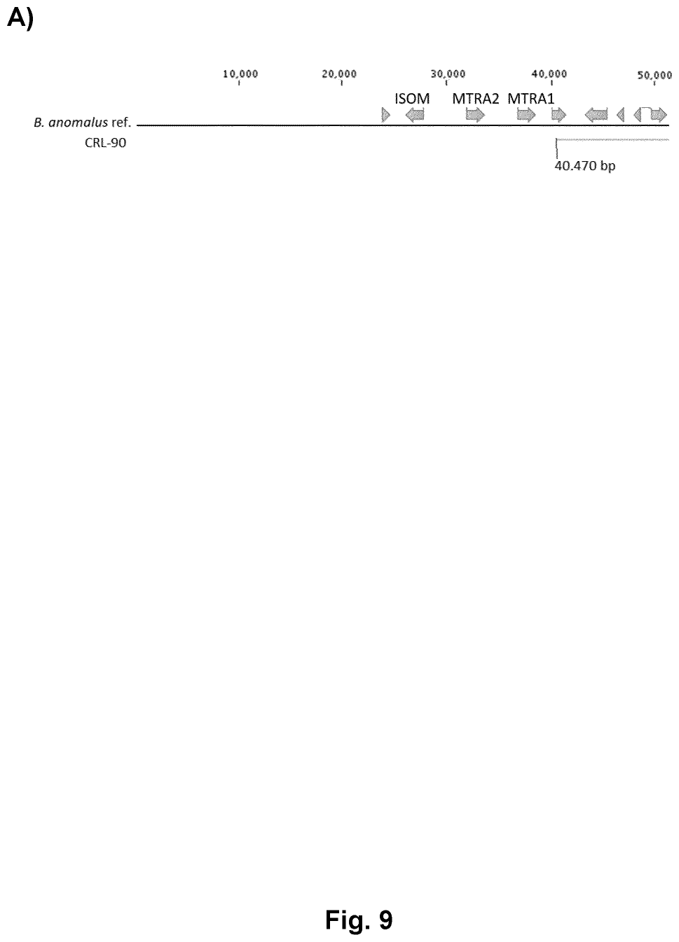

### DETAILED DESCRIPTION OF THE INVENTION

### Definitions

As used herein, “a” can mean one or more, depending on the context in which it is used.

The term “Phenolic off-flavour” or “POF” as used herein refers to a group of phenolic compounds, which can be present in fermented beverages, such as beers. In some types of fermented beverages they are considered as off-flavours and are not desired. Some of them may however be desired in certain types of fermented beverages. Preferably, these compounds are selected from the group of 4-vinylphenol, 4-vinlyguaiacol, 4-ethylphenol and 4-ethylguaiacol.

The term “beer” as used herein refers to a beverage prepared by fermentation of wort. Preferably, said fermentation is done by yeast.

The term “adjunct” as used herein refers to carbon-rich raw material sources added during preparation of a malt and/or cereal based beverage. The adjunct may be an ungerminated cereal grain, which may be milled together with the germinated kernels prepared according to the invention. The adjunct may also be a syrup, sugar or another carbohydrate source.

By the term “wort” is meant a liquid extract of malt and/or cereal kernels and optionally additional adjuncts. Wort is in general obtained by mashing, optionally followed by “sparging”, in a process of extracting residual sugars and other compounds from spent kernels after mashing with hot water. Sparging is typically conducted in a lauter tun, a mash filter, or another apparatus to allow separation of the extracted water from spent kernels. The wort obtained after mashing is generally referred to as “first wort”, while the wort obtained after sparging is generally referred to as the “second wort”. If not specified, the term wort may be first wort, second wort, or a combination of both. During conventional beer production, wort is boiled together with hops. Wort without hops, may also be referred to as “sweet wort”, whereas wort boiled with hops may be referred to as “boiled wort” or simply as wort.

By the term “aqueous extract” as used herein refers to any aqueous extract of malt and/or cereal kernels. Thus, non-limiting examples hereof can be wort or a fermented malt and/or cereal based beverage, such as beer.

The term “aqueous solution” as used herein refers to any aqueous liquids or solutions. The aqueous solution may contain predetermined levels of specific compounds. Thus, non-limiting examples hereof can be any medium, such as medium relevant for yeast strain growth and/or metabolic activity.

The term “°Plato” as used herein refers to density as measured on the Plato scale. The Plato scale is an empirically derived hydrometer scale to measure density of beer or wort in terms of percentage of extract by weight. The scale expresses the density as the percentage of sugar by weight.

The term “fermenting” as used herein is meant to incubate an aqueous extract or aqueous solution with a microorganism, such as a yeast strain.

The term “nitrogen source” as used herein refers to any organic nitrogen containing molecule and/or to ammonium containing molecules. In particular, said nitrogen source may be an organic nitrogen source, for example peptides, amino acids, and/or other amines. The nitrogen source may also be ammonium. Thus, for example N₂ is not considered a “nitrogen source” herein.

The term “malting” as used herein refers to a controlled germination of cereal kernels (in particular barley kernels) taking place under controlled environmental conditions. In some embodiments “malting” may further comprise a step of drying said germinated cereal kernels, e.g. by kiln drying.

The term “malt” as used herein refers to cereal kernels, which have been malted. The term “green malt” refers to germinated cereal kernels, which have not been subjected to a step of kiln drying. In some embodiments the green malt is milled green malt. The term “kiln dried malt” as used herein refers germinated cereal kernels, which have been dried by kiln drying. In some embodiments the kiln dried malt is milled kiln dried malt. In general, said cereal kernels have been germinated under controlled environmental conditions.

The term “Mashing” as used herein refers to the incubation of milled malt (e.g. green malt or kiln dried malt) and/or ungerminated cereal kernels in water. Mashing is preferably performed at specific temperature(s), and in a specific volume of water.

The term “milled” refers to material (e.g. barley kernels or malt), which has been finely divided, e.g. by cutting, milling, grinding or crushing. The barley kernels can be milled while moist using e.g. a grinder or a wet mill. Milled barley kernels or milled malt is sufficiently finely divided to render the material useful for aqueous extracts. Milled barley kernels or milled malt cannot be regenerated into an intact plant by essentially biological methods.

The term “carbon source” as used herein refers to any organic molecule, which can provide energy to yeast and provide carbon for cellular biosynthesis. In particular, said carbon source may be carbohydrates, and more preferably, the carbon source may be monosaccharides, disaccharides trisaccharides and/or tetrasaccharides.

Amino acids may be named herein using the IUPAC one-letter and three-letter codes.

The term “functional homologue” as used herein denotes a polypeptide sharing at least one biological function with a reference polypeptide. In general said functional homologue also shares a significant sequence identity with the reference polypeptide. Preferably a functional homologue of a reference polypeptide is a polypeptide, which has the same biological function as the reference protein and which shares a high level of sequence identity with the reference polypeptide.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. 2011; Li et al. 2015; McWilliam et al., 2013), and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence.

By “encoding” or “encoded”, in the context of a specified nucleic acid, is meant comprising the information for translation into the specified protein. A nucleic acid or polynucleotide encoding a protein may comprise non-translated sequences, e.g. introns, within translated regions of the nucleic acid, or may lack such intervening non-translated sequences, e.g. in cDNA. The information by which a protein is encoded is specified by the use of codons.

As used herein, “expression” in the context of nucleic acids is to be understood as the transcription and accumulation of sense mRNA or antisense RNA derived from a nucleic acid fragment. “Expression” used in the context of proteins refers to translation of mRNA into a polypeptide.

The term “gene” means the segment of DNA involved in producing a polypeptide chain; it includes regions preceding and following a coding region encoding said polypeptide chain (promoter and terminator). Furthermore, some yeast genes also comprise introns although only 5% of the genes in e.g. the S. cerevisiae genome comprise introns. After transcription into RNA, the introns are removed by splicing to generate a mature messenger RNA (mRNA).

The term “mutations” as used herein include insertions, deletions, substitutions, transversions, and point mutations in the coding and noncoding regions of a gene. Point mutations may concern changes of one base pair, and may result in premature stop codons, frameshift mutations, mutation of a splice site or amino acid substitutions. A gene comprising a mutation may be referred to as a “mutant gene”. If said mutant gene encodes a polypeptide with a sequence different to the wild type, said polypeptide may be referred to as a “mutant polypeptide” and/or “mutant protein”. A mutant polypeptide may be described as carrying a mutation, when it comprises an amino acid sequence differing from the wild type sequence.

The term “deletions” as used herein may be a deletion of the entire gene, or of only a portion of the gene, or a part of a chromosome.

The term “splice site” as used herein refers to consensus sequences acting as splice signals for the splicing process. A splice site mutation is a genetic mutation that inserts, deletes or changes a number of nucleotides in the specific site at which splicing takes place during the splicing process, i.e. the processing of precursor messenger RNA into mature messenger RNA (mRNA). Splice site consensus sequences that drive exon recognition are typically located at the very termini of introns.

The term “stop codon” as used herein refers to a nucleotide triplet in the genetic code, which within mRNA results in termination of translation. The term “stop codon” as used herein also refers to a nucleotide triplet within a gene encoding the stop codon in mRNA. The stop codon in DNA typically has one of the following sequences: TAG, TAA or TGA.

The term “growth” as used herein in relation to yeast, refers to the process by which a yeast cells multiply. Thus, when yeast cells are growing, the number of yeast cells increases. The number of yeast cells may be determined by any useful method. Conditions allowing growth of yeast are conditions allowing yeast cells to increase in number. Such conditions in general require the presence of adequate nutrients, e.g. a carbon source and a nitrogen source as well as an adequate temperature, which typically is in the range of 5 to 40° C.

The term “metabolic activity” as used herein refers to yeast strain metabolism, which is normally determined by determining NADH production. Frequently, the metabolic activity correlates with yeast growth and metabolic activity can thus frequently be used as an indicator of yeast growth. No or an insignificant change in metabolism may indicate no growth. NADH production can for example be measured by adding a tetrazolium dye the yeast cells, which is then reduced to purple formazan dependent on NADH production. Metabolic activity can thus be determined based on generation of purple formazan. If the yeast strain has no or very limited metabolic activity, there will be limited NADH production and thus no generation of purple formazan. As described above metabolic activity can frequently be correlated to yeast growth, and if the yeast strain has no growth, there will frequently be insignificant NADH production and thus no generation of purple formazan. The amount of reduced dye, i.e. purple formazan, may be measured using OmniLog® Biolog, which provides an OmniLog Unit, representing cell metabolic activity. A useful method for determining metabolic activity (which as described above frequently may be correlated to yeast growth) is incubating the yeast strain for 80 hours at 25° C. in an aqueous solution containing 10 g/L maltose as a sole carbon source, non-carbohydrate components required for yeast growth and a predetermined level of tetrazolium dye, and determining the formation of purple formazan measured with OmniLog® Biolog. The yeast metabolic activity can then be presented as an absolute Omnilog Unit at a specific time point during incubation or by kinetics presented as OmniLog Units per time, e.g. hours. A yeast strain is considered to have insignificant metabolic activity, and hence frequently such yeast strain is considered not to grow if the OmniLog Unit is below 50 after 80 hours of incubation. In other words, if the slope of the curve showing purple formazan (OmniLog Unit) development over time (hours) is at the most 0.2, such as at the most 0.1, such as at the most 0.05 OmniLog Unit/hour the yeast strain is considered to have insignificant metabolic activity, and hence also considered not able to grow. Another method to quantify the amount of purple formazan is to measure the amount of purple formazan with a spectrophotometer at a wavelength of 590 nm.

The term “yeast strain is not capable of utilizing XX as sole carbon source” as used herein refers to a yeast strain, which cannot grow and/or which has insignificant metabolic activity when incubated with a medium containing XX as the only carbon source, wherein “XX” may be any specific carbon source, e.g. a sugar. “Carbon sources” may in particular be carbohydrates. Thus, said medium preferably does not contain any other carbohydrates apart from XX. For example, the yeast strain may not be capable of utilizing maltose as sole carbon source.

The term “low-alcohol beverage” is used herein to describe a fermented malt and/or cereal based beverage with an ethanol content below 3%. Preferably, a “low-alcohol beverage” may have an ethanol content below 2%. The low alcohol-beverage may for example be a low-alcohol beer, with an ethanol content below 3%, preferably below 2%.

The terms “alcohol-free beverage” or “non-alcohol beverage” herein are used herein to describe a fermented malt and/or cereal based beverage with an ethanol content of no more than 0.5%. The alcohol-free beverage may for example be an alcohol-free beer and the non-alcohol beverage may for example be a non-alcohol beer, with an ethanol content below 0.5%.

### Properties of Yeast

The present invention relates to a Dekkera yeast strain having at least one of the characteristics I, II, and III described herein below. Besides characteristics I, II, and III said yeast strain may have one or more of the characteristics selected from the group consisting of characteristics IV, V, VI and VII. In addition said Dekkera yeast strain may have one or more of the genotypes I, II, III, IV, V, VI, VII, VIII, IX, X as described below.

The term Dekkera as used herein may refer both to teleomorph Dekkera strains as well as to anamorph Brettanomyces strains.

The term Dekkera is sometimes used interchangeably with the term Brettanomyces. Sometimes, the term “Brettanomyces” is used to designate an anamorph or non-spore forming yeast of the genus Dekkera, whereas the term “Dekkera” may be used to describe the teleomorph or spore forming form of the yeast.

The genus Dekkera may in particular comprise the teleomorph yeast strains Dekkera anomala and Dekkera bruxellensis. Brettanomyces may in particular comprise the anamorph forms of Dekkera, namely Brettanomyces nanus, Brettanomyces naardenensis, Brettanomyces custerisianus, Brettanomyces anomalus and/or Brettanomyces bruxellensis. Preferably the yeast strain of the invention is a yeast strain of a species selected from the group consisting of Dekkera anomalus and Dekkera bruxellensis. However, as noted above, the terms Dekkera and Brettanomyces are sometimes used interchangeably. Thus, Dekkera anomalus and Dekkera bruxellensis are also known as Brettanomyces bruxellensis and Brettanomyces anomalus, respectively, wherein the former term may designate the teleomorph form and the latter may refer to the anamorph form. Herein, the term “Dekkera” covers both the Dekkera and the Brettanomyces forms of the yeast.

In one embodiment said yeast strain has characteristic I described herein below. In another embodiment, said yeast strain has characteristic II described herein below.

In particular it is preferred that said yeast strain at least has characteristics I and II described herein below.

In another embodiment, said yeast strain may also have characteristics I and III, or characteristics II and III, or characteristics I, II and III described herein below.

In another embodiment, the yeast strain according to the present invention has characteristic I, II and/or III described herein below and furthermore has one or more of characteristics IV, V, VI and VII as described herein below.

### Characteristic I

The invention relates to a Dekkera yeast strain with reduced ability to convert p-coumaric acid into 4-ethylphenol and methods of producing beverages using said yeast. Thus, Dekkera yeast strain of the invention may have the characteristic I, wherein characteristic I is reduced ability to convert p-coumaric acid into 4-ethylphenol. In particular, characteristic I is that said yeast strain is not capable of converting more than 25% p-coumaric acid into 4-ethylphenol.

In embodiments of the invention, wherein the Dekkera yeast strain according to the invention has the characteristic I, said Dekkera yeast strain in general also has genotype I and/or genotype II. Preferably said yeast strain has genotype 1.

The Dekkera yeast strain of the invention has a reduced ability to convert p-coumaric acid into 4-ethylphenol. Without being bound by theory it is believed that conventional Dekkera yeast strains may contain enzymatic activities catalyzing the following reactions:

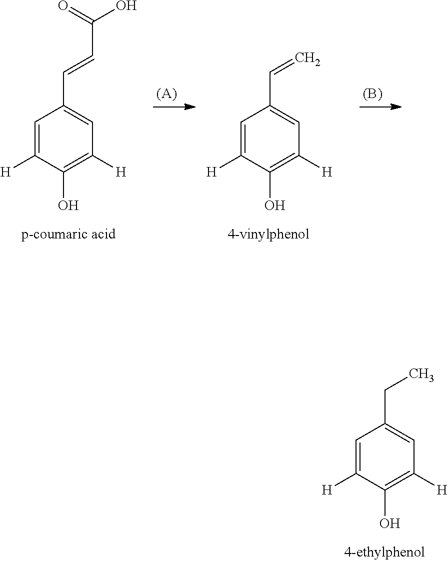

Accordingly, the Dekkera yeast strain of the invention may for example have a reduced ability to convert p-coumaric acid to 4-vinylphenol and/or the Dekkera yeast strain of the invention may have a reduced ability to convert 4-vinylphenol to 4-ethylphenol.

It is preferred that the Dekkera yeast strain of the invention is not capable of converting more than 25% of the p-coumaric acid present in an aqueous solution into 4-ethylphenol, when incubated in said aqueous solution. For example, the Dekkera yeast strain of the invention may not be capable of converting more than 20%, such as not more than 15%, for example not more than 10%, such as not more than 5%, for example not more than 1% of the p-coumaric acid present in an aqueous solution into 4-ethylphenol, when incubated in said aqueous solution.

Whether said Dekkera yeast strain is capable of converting the p-coumaric acid present in an aqueous solution into 4-ethylphenol may be determined in different manners. In one embodiment it is determined by a method comprising the steps of:
providing an aqueous solution containing a predetermined level of p-coumaric acid
incubating the Dekkera yeast strain to be tested with said aqueous solution
determining the level of p-coumaric acid in the aqueous solution subsequent to said incubation
wherein the reduction in p-coumaric acid level is considered a measure of the conversion of p-coumaric acid to 4-ethylphenol.

Accordingly, it is preferred that when the Dekkera yeast strain according to the invention is incubated in an aqueous solution containing a predetermined level of p-coumaric acid, then the level of p-coumaric subsequent to said incubation is at the most 25%, such as the most 20%, such as at the most 15%, for example at the most 10%, such as at the most 5%, for example at the most 1% lower than the starting level.

In one embodiment, whether said Dekkera yeast strain is capable of converting the p-coumaric acid present in an aqueous solution into 4-ethylphenol is determined by a method comprising the steps of:
providing an aqueous solution containing p-coumaric acid and a predetermined level of 4-ethylphenol
incubating the Dekkera yeast stain to be tested with said aqueous solution
determining the level of 4-ethylphenol in the aqueous solution subsequent to said incubation
wherein the increase in 4-ethylphenol is considered a measure of the conversion of p-coumaric acid to 4-ethylphenol.

Accordingly, it is preferred that when the Dekkera yeast strain according to the invention is incubated in an aqueous solution containing a predetermined level of p-coumaric acid and a predetermined level of 4-ethylphenol, then the molar increase in the 4-ethylphenol level after incubation is at the most 25%, such as at the most 20%, such as at the most 15%, for example at the most 10%, such as at the most 5%, for example at the most 1% of the predetermined molar level of p-coumaric acid.

Regardless of whether the method of determining whether said Dekkera yeast strain is capable of converting the p-coumaric acid present in an aqueous solution into 4-ethylphenol involves determined level of p-coumaric acid or the level of 4-ethylphenol, then the incubation in aqueous solution may be performed in any suitable manner. In general, the incubation is made under conditions allowing growth and/or metabolic activity of said Dekkera yeast strain. Thus, the incubation is performed at a temperature in the range of 5 to 30° C., such as in the range of 15 to 25° C. The aqueous solution should in addition to p-coumaric acid also comprise components promoting yeast strain growth including a carbon source and a nitrogen source and optionally buffer and salts. Thus, the aqueous solution may for example be a synthetic medium, such as YPD supplemented with glucose and p-coumaric acid. Alternatively, the aqueous solution may be wort. The incubation may for example be done for 3 to 7 days.

In one preferred embodiment, whether a Dekkera yeast strain is capable of converting the p-coumaric acid present in an aqueous solution into 4-ethylphenol is determined by the method described in Example 2 below.

In another embodiment of the present invention, said Dekkera yeast strain can also have a reduced ability to convert p-coumaric acid into 4-vinylphenol. Thus, Dekkera yeast strain of the invention can have the characteristic I, wherein characteristic I is also characterized by having a reduced ability to convert p-coumaric acid into 4-vinylphenol. In particular, characteristic I also covers a yeast strain which is not capable of converting more than 25% such as not more than 20%, such as not more than 15%, such as not more than 10%, such as not more than 5%, such as not more than 1% of the p-coumaric acid present in the aqueous solution into 4-vinylphenol.

Whether said Dekkera yeast strain is capable of converting the p-coumaric acid present in an aqueous solution into 4-vinylphenol may be determined by a method comprising the steps of:
providing an aqueous solution containing p-coumaric acid and a predetermined level of 4-vinylphenol
incubating the Dekkera yeast stain to be tested with said aqueous solution
determining the level of 4-vinylphenol in the aqueous solution subsequent to said incubation
wherein the increase in 4-vinylphenol is considered a measure of the conversion of p-coumaric acid to 4-vinylphenol.

Accordingly, it is preferred that when the Dekkera yeast strain according to the invention is incubated in an aqueous solution containing a predetermined level of p-coumaric acid and a predetermined level of 4-vinylphenol, then the molar increase in the 4-vinylphenol level after incubation is at the most 25%, such as at the most 20%, such as at the most 15%, for example at the most 10%, such as at the most 5%, for example at the most 1% of the predetermined molar level of p-coumaric acid.

Incubation of said Dekkera yeast strain in an aqueous solution may be performed in any suitable manner, such as described herein above.

### Characteristic II

The Dekkera yeast strain of the invention may have the characteristic II, wherein characteristic II is reduced ability to convert ferulic acid into 4-ethylguaiacol. In particular, the yeast strain of the invention may have characteristic II in addition to characteristic I (not capable of converting more than 25% p-coumaric acid into 4-ethylphenol).

In embodiments of the invention, wherein the Dekkera yeast strain according to the invention has the characteristic II, said Dekkera yeast strain in general also has genotype I and/or genotype II. Preferably said yeast strain has genotype 1.

In embodiments of the present invention, the Dekkera yeast strain of the invention may for example have a reduced ability to convert ferulic acid to 4-vinylguaiacol and/or the Dekkera yeast strain of the invention may have a reduced ability to convert 4-vinylguaiacol to 4-ethylguaiacol.

Thus, the Dekkera yeast strain of the invention may have characteristic II, wherein characteristic II is that the Dekkera yeast strain is not capable of converting more than 25% of the ferulic acid present in an aqueous solution into 4-ethylguaiacol, when incubated in said aqueous solution. For example, the Dekkera yeast strain of the invention may not be capable of converting more than 20%, such as not more than 15%, for example not more than 10%, such as not more than 5%, for example not more than 1% of the ferulic acid present in an aqueous solution into 4-ethylguaiacol, when incubated in said aqueous solution.

Whether said Dekkera yeast strain is capable of converting the ferulic acid is present in an aqueous solution into 4-ethylguaiacol may be determined essentially as described herein above in relation to characteristic I except that the levels of ferulic acid and/or 4-ethylguaiacol is determined.

In one preferred embodiment, whether a Dekkera yeast strain is capable of converting the ferulic acid present in an aqueous solution into 4-ethylguaiacol is determined by the method described in Example 2 below.

In another embodiment of the present invention, said Dekkera yeast strain can also have a reduced ability to convert ferulic acid into 4-vinylguaiacol. Thus, Dekkera yeast strain of the invention can have the characteristic II, wherein characteristic II is also characterized by having a reduced ability to convert ferulic acid into 4-vinylguaiacol. In particular, characteristic II also covers a yeast strain which is not capable of converting more than 25% such as not more than 20%, such as not more than 15%, such as not more than 10%, such as not more than 5%, such as not more than 1% of the ferulic acid present in the aqueous solution into 4-vinylguaiacol.

Whether said Dekkera yeast strain is capable of converting the ferulic acid present in an aqueous solution into 4-vinylguaiacol may be determined by a method comprising the steps of:
providing an aqueous solution containing ferulic acid and a predetermined level of 4-vinylguaiacol
incubating the Dekkera yeast stain to be tested with said aqueous solution
determining the level of 4-vinylguaiacol in the aqueous solution subsequent to said incubation
   wherein the increase in 4-vinylguaiacol is considered a measure of the conversion of p-coumaric acid to 4-vinylguaiacol.

Accordingly, it is preferred that when the Dekkera yeast strain according to the invention is incubated in an aqueous solution containing a predetermined level of ferulic acid and a predetermined level of 4-vinylguaiacol, then the molar increase in the 4-vinylguaiacol level after incubation is at the most 25%, such as at the most 20%, such as at the most 15%, for example at the most 10%, such as at the most 5%, for example at the most 1% of the predetermined molar level of ferulic acid.

Incubation of said Dekkera yeast strain in an aqueous solution may be performed in any suitable manner, such as described herein above.

### Characteristic III

The Dekkera yeast strain of the invention may also have characteristic III, wherein characteristic III is that the Dekkera yeast strain is not capable of utilizing more than 2% maltose. In one embodiment of the present invention, the yeast strain is not capable of utilizing more than 1.5%, such as 1%, such as 0.1% maltose.

In other words, the present invention relates to a Dekkera yeast strain, which is not capable of utilizing more than 20 g/L maltose. In one embodiment of the present invention, the yeast strain is not capable of utilizing more than 15 g/L such as 10 g/L, such as 1 g/L maltose.

In embodiments of the invention, wherein the Dekkera yeast strain according to the invention has the characteristic III, said Dekkera yeast strain in general also has one or more of genotypes III, IV and V. Preferably said yeast strain has all of genotypes III, IV and V.

The ability of the yeast strain to utilize maltose can be calculated using different methods. One method is to measure the amount of maltose present in an aqueous extract or an aqueous solution comprising maltose before incubation of the aqueous extract or aqueous solution with the yeast strain and after incubation with the yeast strain, and calculate the difference in the amount of the maltose before and after incubation with the yeast strain. The incubation of the aqueous extract with the yeast strain might for example be at 5 to 30° C., such as at 10 to 28° C., such as at 15 to 25° C., for 1 to 21 days, e.g. for 2 to 10 days, e.g. for 3 to 7 days. The incubation of the aqueous solution with the yeast strain might for example be at 15 to 35° C., such as 20 to 30° C., for 1 to 80 hours, such as 60 to 80 hours. The difference in the amount of maltose may for example be used to calculate the absolute amount of maltose in e.g. g/kg or g/L, which the yeast strain has utilized or calculate it as a % (e.g. w/w) utilized maltose.

In one embodiment of the present invention, said yeast strain is not capable of utilizing more than 2% maltose, when incubated in an aqueous solution comprising maltose and glucose.

Preferably, said yeast strain is not able to utilize more than 1.5%, such as 1%, such as 0.1% maltose when incubated in an aqueous solution comprising maltose and glucose. Said aqueous extract may in particular be wort. Incubation of said yeast strain in said aqueous extract may for example be at 5 to 30° C., such as at 10 to 28° C., such as at 15 to 25° C., for 1 to 21 days, e.g. for 3 to 7 days. The aqueous extract may for example contain more than 40 g/kg maltose. In one embodiment, the aqueous solution may contain in the range of 40 to 100 g/kg maltose. The aqueous solution may in some embodiments of the invention for example contain in the range of 4 to 50 g/kg glucose.

Preferably, the yeast strain according to the invention is not capable of utilizing more than 2%, such as not more than 1% of the maltose when incubated at 25° C. for 10 days in an aqueous solution comprising in the range of 40 to 100 g/kg maltose and in the range of 8 to 50 g/kg glucose. Very preferably, the yeast strain according to the invention is not be capable of utilizing more than 2%, such as not more than 1% of the maltose when determined by fermenting wort as described herein below in Example 5.

When determining whether a yeast strain is capable of utilizing maltose it is generally preferred to use a method for determining maltose concentration, wherein the method has an uncertainty of measurement which is significantly less than 2% in relation to the total maltose concentration. This may for example be achieved by using an average of multiple measurements, e.g. of at least 10 independent measurements.

In one embodiment of the present invention, the yeast strain according to the invention is not capable of utilizing any of the maltose present in the aqueous solution. In such embodiments, for example, the amount of the maltose present in the aqueous solution after incubation with the yeast strain will not be less than the amount of the maltose present in the aqueous extract before incubation with the yeast strain.

In one embodiment of the present invention, the yeast strain is not capable utilizing maltose as sole carbon source. Thus, it is preferred that the yeast strain is not capable of growing and/or has insignificant metabolic activity in an aqueous solution containing maltose as the sole carbon source. Such aqueous solution preferably do not contain any monosaccharides, disaccharides, trisaccharides and/or tetrasaccharides apart from maltose, and more preferably such aqueous solution does not contain any carbohydrates apart from maltose. For example, a yeast strain is considered to have insignificant metabolic activity, when insignificant metabolic activity is determined as described in Example 4 below.

In one embodiment, the yeast strain of the present invention is not capable of growing and/or has insignificant metabolism when incubated in a aqueous solution containing in the range of 5 to 15 g/L maltose, for example in the range of 8 to 12 g/L maltose, wherein maltose is the sole carbon source. Such aqueous solution preferably do not contain any carbohydrates apart from said concentration of maltose. The incubation period may be for 1 to 80 hours, such as 60 to 80 hours, at e.g. 15 to 35° C., such as 20 to 30° C. For example, a yeast strain is considered to have insignificant metabolic activity, when insignificant metabolic activity is determined as described in Example 4 below.

Yeast strain growth can be measured using different methods. In one embodiment yeast strain growth is determined by a method comprising the steps of:
providing an aqueous solution containing in the range of 5 to 15 g/L maltose as a sole carbon source,
incubating said aqueous solution with a predetermined number of yeast cells of said yeast strain according to the invention for 60 to 80 hours at 20 to 30° C.
determined the number of yeast cells in the aqueous solution

The number of yeast cells can be determined by any suitable method known in the art.

In one embodiment of the present invention, the yeast strain growth is correlated to metabolic activity. In such cases, growth is determined indirectly by determining metabolic activity. Metabolic activity can for example be determined by a method comprising the steps of
providing an aqueous solution containing in the range of 5 to 15 g/L maltose as a sole carbon source and a predetermined level of a compound (e.g. tetrazolium), which respond to NADH production by being reduced to a dye (e.g. purple formazan),
incubating said aqueous solution with said yeast strain according to the invention for 60 to 80 hours at 20 to 30° C.
quantify the amount of reduced dye (e.g. purple formazan) in the aqueous solution.

Preferably, the test for yeast cell growth and/or metabolic activity is performed in replicates, such as duplicates, or triplicates etc. Thus, the steps of the method may preferably be performed one or more times, such as 2 or more times, such as 3 or more times, such as 10 or more times for each tested yeast strain. The average growth and/or metabolic activity of the yeast strain may be calculated as the average amount of reduced dye within the tested yeast strain replicates.

Several methods can be used to measure the amount of reduced dye (e.g. purple formazan).

Accordingly, when said yeast strain according to the invention is incubated in an aqueous solution containing in the range of 5 to 15 g/L maltose as a sole carbon source, non-carbohydrate components required for yeast growth and a predetermined level of dye responding to cellular NADH production, for 60 to 80 hours at 20 to 30° C., then said yeast strain is not capable of growing and/or is considered to have insignificant metabolic activity, when the amount of reduced dye, measured with OmniLog® Biolog is at the most 50 OmniLog Units, such as at the most 40 OmniLog Units.

In one embodiment, said yeast strain according to the invention is not capable of growing and/or is considered to have insignificant metabolic activity when incubated for 80 hours at 25° C. in an aqueous solution containing 10 g/L maltose as a sole carbon source, non-carbohydrate components required for yeast growth and a predetermined level of tetrazolium dye, wherein said yeast strain is considered not capable of growing and/or is considered to have insignificant metabolic activity when the formation of purple formazan measured with OmniLog® Biolog is at the most 50 OmniLog Units after 80 hours.

In another embodiment, the growth of said tested yeast strain is measured based on growth kinetics of said yeast strain during the incubation period. Thus, the amount of reduced dye can be quantified and plotted against the incubation time whereby it is possible to calculate the slope of the curve showing the amount of reduced dye overtime.

Accordingly, when said yeast strain according to the invention is incubated in an aqueous solution containing in the range of 5 to 15 g/L maltose as a sole carbon source, non-carbohydrate components required for yeast growth and a predetermined level of dye responding to cellular NADH production, for 60 to 80 hours at 20 to 30° C., then said yeast strain is not capable of growing and/or is considered to have insignificant metabolic activity, when the slope of the curve showing the amount of reduced dye, measured with OmniLog® Biolog over time is less than 0.2, such as less than 0.1, such as less than 0.05 OmniLog Unit/hour.

In one embodiment, said yeast strain according to the invention is not capable of growing and/or is considered to have insignificant metabolic activity, when incubated for 80 hours at 25° C. in an aqueous solution containing 10 g/L maltose as a sole carbon source, non-carbohydrate components required for yeast growth and a predetermined level of tetrazolium dye, wherein said yeast strain is considered not capable of growing and/or is considered to have insignificant metabolic activity, when the slope of the curve showing purple formazan measured with OmniLog® Biolog over time is at the most 0.2, such as at the most 0.1, such as at the most 0.05 OmniLog Unit/hour.

Another non-limiting method of quantifying the amount of reduced dye, is to measure the amount of reduced dye by using a spectrophotometer. Thus, one example hereof is to measure the amount of formazan with a spectrophotometer at a wavelength of 590 nm.

In one embodiment, said yeast strain according to the invention is incubated in an aqueous solution containing in the range of 5 to 15 g/L maltose as a sole carbon source, non-carbohydrate components required for yeast growth and a predetermined level of dye responding to NADH production, for 60 to 80 hours at 20 to 30° C., said yeast strain according to the invention is considered not capable of growing and/or is considered to have insignificant metabolic activity, when the reduced dye measured at a wavelength of 590 nm with a spectrophotometer does not increase more than 2-fold after 80 hours.

In one embodiment, said yeast strain according to the invention is not capable of growing and/or is considered to have insignificant metabolic activity when incubated for 80 hours at 25° C. in an aqueous solution containing 10 g/L maltose as a sole carbon source, non-carbohydrate components required for yeast growth and a predetermined level of tetrazolium dye, wherein said yeast strain is considered not capable of growing and/or is considered to have insignificant metabolic activity when the formation of purple formazan measured at a wavelength of 590 nm with a spectrophotometer does not increase more than 2-fold after 80 hours.

### Characteristics IV

The Dekkera yeast strain according to the present invention may also have characteristic IV, wherein characteristic IV is that the Dekkera yeast strain is not capable of utilizing more than 5% maltotriose. In one embodiment of the present invention, the yeast strain is not capable of utilizing more than 4% maltotriose, such as 3%, such as 2%, such as 1%, such as 0.1% maltotriose.

Thus, upon incubation in an aqueous extract containing maltotriose, then said yeast strain is not capable of utilizing more than 5% of said maltotriose. Preferably, said yeast strain is not able to utilize more than 1.5%, such as 1%, such as 0.1% of said maltotriose present in the aqueous extract. Said aqueous extract may in particular be wort. Incubation of said yeast strain in said aqueous extract may for example be at 5 to 25° C., such as 10 to 20° C., for 1 to 21 days, e.g. for 3 to 7 days. The amount of maltotriose in the aqueous extract may for example be 1 to 50 g/kg, such as 10 to 20 g/L.

The capability of the yeast strain not to utilize maltotriose can be calculated as described above for maltose.

One useful method for determining whether a yeast strain is not capable of utilizing maltotriose in wort is described in Example 5.

### Characteristics V

The Dekkera yeast strain according to the present invention may also have characteristic V, wherein characteristic V is that the Dekkera yeast strain is not capable of utilizing more than 5% maltotetraose. In one embodiment of the present invention, the yeast strain is not capable of utilizing more than 4% maltotetraose, such as 3%, such as 2%, such as 1%, such as 0.1% maltotetraose.

Thus, upon incubation in an aqueous extract containing maltotetraose, then said yeast strain is not capable of utilizing more than 5% of said maltotetraose. Preferably, said yeast strain is not able to utilize more than 1.5%, such as 1%, such as 0.1% of said maltotriose present in the aqueous extract. Said aqueous extract may in particular be wort. Incubation of said yeast strain in said aqueous extract may for example be at 5 to 25° C., such as 16 to 18° C., for 1 to 21 days, e.g. for 3 to 7 days. The amount of maltotriose in the aqueous extract may for example be 0.5 to 15 g/kg, such as 1 to 5 g/L.

The capability of the yeast strain not to utilize maltotetraose can be calculated as described above for maltose.

One useful method for determining whether a yeast strain is not capable of utilizing maltotetraose in wort is described in Example 5.

### Characteristics VI

The Dekkera yeast strain according to the present invention may also have characteristic VI, wherein characteristic VI is that the Dekkera yeast strain is not capable of utilizing glucose. Thus, upon incubation in an aqueous extract containing glucose, then said yeast strain is capable of utilizing a part of the glucose present in the aqueous extract.

More preferably, the yeast strain is capable of utilizing glucose as the sole carbon source. Thus, the yeast strain is capable of growing in a medium containing glucose as the sole carbon source. Such medium preferably do not contain any monosaccharides, disaccharides, trisaccharides and/or tetrasaccharides apart from glucose, and more preferably such medium does not contain any carbohydrates apart from glucose. One useful method for determining whether a yeast strain is capable of utilizing glucose as a sole carbon source is described in Example 4.

The skilled person will understand that the methods described in Example 4 can be used to test whether the yeast strain is capable of growing in a medium containing glucose or maltose as a sole carbon source, and that the method described in Example 5 can be used to test whether the yeast strain is capable of utilizing fermentable sugars such as maltose, maltotriose, maltotetraose, and glucose, present in an aqueous extract, such as wort.

### Characteristics VII

The Dekkera yeast strain according to the present invention may also have characteristic VII, wherein characteristic VII is that the Dekkera yeast strain has a low production of ethanol. Since the amount of ethanol produced by a given yeast strain is highly influenced by the starting material, it is preferred that the yeast strain is not capable of generating more than 1.5 promille ethanol per °Plato, such as 1.3 promille ethanol per °Plato, such as 1.1 promille ethanol per °Plato. °Plato is a measure for the density of a liquid, and thus indicates the level of sugars and other fermentable nutrients.

In one embodiment, it is preferred that the yeast strain is not capable of generating more than 1.5 promille ethanol per °Plato, when said yeast strain is added to an aqueous extract having a sugar content of at the most 10° Plato, such as of the most °Plato. In particular, the yeast strain is not capable of generating more than 1.5 promille ethanol per °Plato, when said yeast strain is added to an aqueous extract comprising glucose and maltose. The aqueous extract may contain more than 40 g/kg maltose. In one embodiment, the aqueous solution may contain in the range of 40 to 100 g/kg maltose. In one embodiment, the aqueous extract may for example contain at the most 15 g/kg glucose, such as at the most 10 g/kg glucose, for example at the most 5 g/kg glucose.

In one embodiment of the present invention, the Dekkera yeast strain, is not capable of producing more than 2% ethanol. In another embodiment of the present invention, the yeast strain is not capable of producing more than 1.5% ethanol. Thus, upon incubation in an aqueous extract comprising maltose and glucose, then said yeast strain is not capable of producing more than 2% ethanol, such as no more than 1.5% ethanol. Said aqueous extract may in particular be wort. Incubation of said yeast strain in said aqueous extract may for example be at 5 to 25° C., such as 10 to 20° C., for 1 to 21 days, e.g. for 3 to 7 days. The amount of maltose in the aqueous extract may for example be 5 to 200 g/kg, such as 40 to 70 g/kg, such as 50 to 60 g/kg. The aqueous extract may contain at the most 15 g/kg glucose, such as at the most 10 g/kg glucose. In one example, said yeast strain may not be capable of producing more than 2% ethanol, when incubated in an aqueous extract comprising 50 to 60 g/kg maltose and 9 to 11 g/kg glucose, as described herein below in Example 5.

### Species

The yeast strain may be any Dekkera yeast strain. If nothing else is specified, the term “Dekkera” will in this application cover both the Dekkera (e.g. the teleomorph forms) and the Brettanomyces (e.g. the anamorph forms) of the yeast.

In preferred embodiments, the yeast strain is of the species Dekkera anomalus, Dekkera bruxellensis, Brettanomyces anomalus, or Brettanomyces bruxellensis. In particular, the yeast strain may be of the species Dekkera bruxellensis or Dekkera anomalus, which both are found to produce a unique and desirable flavor profile during fermentation, compared to other Dekkera species. In a preferred embodiment, the yeast strain is a Dekkera anomalus. Dekkera anomalus is also known as Dekkera claussenii.

### Genetic Background

### Gene Mappinq

Whole-genome sequencing were performed for Dekkera yeast strains.

CRL-49 (Dekkera anomalus) was used herein as a reference for D. anomalus. The genome of the D. bruxellensis UMY321 isolate served as a reference for D. bruxellensis. UMY321 is publicly available from NCBI.

All the open reading frames of the genomes were identified and the putative function of each gene were based on comparison to the UniprotKB and Pfam databases using Blastp and HMMER respectively. The putative function of the predicted genes responsible for maltose assimilation, has not previously been proven.

Two genes potentially responsible for POF-production were identified in Dekkera, one decarboxylase, denoted “DPAD” herein and one superoxide dismutase, denoted “DSOD” herein. Dekkara bruxellensis comprises two PAD genes, DbPAD1 and DbPAD2. If not specified otherwise the term PAD in respect of Dekkara bruxellensis refers to DbPAD2. The sequences of the Dekkera PAD and SOD genes and polypeptides are provided herein as follows:
DaPAD1 (SEQ ID NO:1) encoding a DaPAD1 protein of SEQ ID NO:2
DaSOD (SEQ ID NO:3) encoding a DaSOD protein of SEQ ID NO:4
DbPAD1 (SEQ ID NO:23) encoding a DbPAD1 protein of SEQ ID NO:24
DbPAD2 (SEQ ID NO:5) encoding an off-frame DbPAD2 protein of SEQ ID NO:6
DbSOD (SEQ ID NO:7) encoding a DbSOD protein of SEQ ID NO:8

Genes potentially responsible for maltose assimilation were identified in Dekkera:

This includes maltose transporters, denoted “MTRA” herein and the major isomaltase, denoted “ISOM” herein:
DaMTRA1 (SEQ ID NO:9) encoding a DaMTRA1 protein of SEQ ID NO:10
DaISOM (SEQ ID NO:11) encoding a DaISOM protein of SEQ ID NO:12
DaMTRA2 (SEQ ID NO:13) encoding a DaMTRA2 protein of SEQ ID NO:14
DbMTRA1 (SEQ ID NO:15) encoding a DbMTRA1 protein of SEQ ID NO:16
DbISOM(2) (SEQ ID NO:17) encoding a DbISOM(2) protein of SEQ ID NO:18
DbMTRA2 (SEQ ID NO:19) encoding a DbMTRA2 protein of SEQ ID NO:20
DbISOM(1) (SEQ ID NO:21) encoding a DbISOM(1) protein of SEQ ID NO:22
DbMTRA3 (SEQ ID NO:25) encoding a DbMTRA3 protein of SEQ ID NO:26
DbMTRA4 (SEQ ID NO:27) encoding a DbMTRA4 protein of SEQ ID NO:28
DbMTRA5 (SEQ ID NO:29) encoding a DbMTRA5 protein of SEQ ID NO:30
DbMTRA6 (SEQ ID NO:31) encoding a DbMTRA6 protein of SEQ ID NO:32

The maltose assimilation genes are distributed across the genome, with a main cluster containing the enzyme ISOM surrounded by the maltose transporters (MTRA1, MTRA2, MTRA3, MTRA4) present in scaffold I, i.e. herein named MAL loci.

### Genotype—Phenotype

The Dekkera yeast strain according to the invention may have one or more of the phenotypic characteristics I to III described herein above. In addition to the phenotypic characteristic I to III or alternatively the yeast strain may have one or more characteristics selected from the group consisting of characteristics IV, V, VI and VII.

In addition to said phenotypic characteristics, the yeast strain according to the invention may have one or more of the genotypes I to X described herein below. Said genotypes may be linked to the phenotypic characteristics I to III outlined above as well as the phenotypic characteristics IV to VII outlined above.

In one embodiment, the yeast strain according to the invention at least has the genotype I described herein below. In addition to having genotype I said yeast may also have one or more of the genotypes II to V and one or more of the phenotypic characteristics described above.

Thus, in one embodiment of the invention, the yeast strain has at least the genotype I described below and the genotype II described below. In addition to having the genotypes I and II, said yeast may also have one or more of the genotypes III to V and one or more of the characteristics I to III.

In another embodiment, the yeast strain may have an additional genotype and phenotype described herein below.

### Genotype I: PAD

The Dekkera yeast strain according to the invention may have the genotype I, wherein the genotype I is the presence of one or more mutations in or a deletion of the gene encoding PAD. In embodiments of the invention, wherein the Dekkera yeast strain according to the invention has the genotype I, said Dekkera yeast strain in general also have characteristic I and/or II, preferably said yeast strain has both of characteristics I and II.

The gene encoding functional PAD is herein denoted PAD1 in Dekkera anomalus whereas it is denoted PAD2 in Dekkera bruxellensis. Accordingly, the genotype I may be the presence of one or more mutations in or a deletion of the gene encoding PAD2 of Dekkera bruxellensis or the gene encoding PAD1 of Dekkera anomalus.

In one embodiment, wherein the yeast strain is a Dekkera anomalus yeast strain, said yeast strain has the genotype I, wherein genotype I is that said yeast strain comprises a mutation in or a deletion of the gene encoding DaPAD1 of SEQ ID NO:2 or a functional homologue thereof having at least 80% sequence identity herewith.

In another embodiment, wherein the yeast strain is a Dekkera bruxellensis yeast strain, said yeast strain has the genotype I, wherein genotype I is that said yeast strain comprises a mutation in or a deletion of the gene encoding DbPAD2 of SEQ ID NO:6 or a functional homologue thereof having at least 80% sequence identity herewith.

PAD may be responsible for the decarboxylation of p-coumaric acid into 4-vinylphenol, as well as the decarboxylation of ferulic acid present into 4-vinylguaiacol.

In one embodiment of the present invention, the yeast strain according to the invention lacks the gene encoding PAD. Thus, the yeast strain may have a deletion of the gene encoding PAD.

In another embodiment, wherein the yeast strain is a Dekkera anomalus yeast strain, said yeast strain according to the invention lacks the gene encoding DaPAD1 of SEQ ID NO:2 or a functional homologue thereof having at least 80% sequence identity herewith. In other words, the yeast strain of the species Dekkera anomalus may have a deletion of the gene encoding DaPAD1 of SEQ ID NO:2 or a functional homologue thereof having at least 80%, such as at least 90%, for example at least 95% sequence identity herewith. In particular, said yeast strain of the species Dekkera anomalus may have a deletion of the gene encoding DaPAD1 of SEQ ID NO:2.

In yet another embodiment, wherein the yeast strain is a Dekkera bruxellensis yeast strain, said yeast strain according to the invention lacks the gene encoding DbPAD2 of SEQ ID NO:6 or a functional homologue thereof having at least 80% sequence identity herewith. In other words, the yeast strain of the species Dekkera bruxellensis may have a deletion of the gene encoding DbPAD2 of SEQ ID NO:6 or a functional homologue thereof having at least 80%, such as at least 90%, for example at least 95% sequence identity herewith.

In one embodiment, the yeast strain according to the present invention comprises one or more deletions in the gene encoding PAD so that said gene encodes mutant PAD polypeptide lacking at least some of PAD, such as lacking at least 10% of the amino acids of PAD, such as lacking at least 20%, such as lacking at least 30%, such as lacking at least 40%, such as lacking at least 50%, such as lacking at least 60%, such as lacking at least 70%, such as lacking at least 80%, such as lacking at least 90% of the amino acids of PAD.

In another embodiment, wherein the yeast strain is a Dekkera anomalus yeast strain, said yeast strain lacks a portion of the gene encoding DaPAD1, such as lacking at least 10% of the amino acids of DaPAD1, such as lacking at least 20%, such as lacking at least 30%, such as lacking at least 40%, such as lacking at least 50%, such as lacking at least 60%, such as lacking at least 70%, such as lacking at least 80%, such as lacking at least 90% of the amino acids of DaPAD1 of SEQ ID NO:2 or a functional homologue thereof having at least 80%, such as at least 90%, for example at least 95% sequence identity herewith.

In yet another embodiment, wherein the yeast strain is a Dekkera bruxellensis yeast strain, said yeast strain lacks a portion of the gene encoding DbPAD2, such as lacking at least 10% of the amino acids of DbPAD2, such as lacking at least 20%, such as lacking at least 30%, such as lacking at least 40%, such as lacking at least 50%, such as lacking at least 60%, such as lacking at least 70%, such as lacking at least 80%, such as lacking at least 90% of the amino acids of DbPAD2 of SEQ ID NO:6 or a functional homologue thereof having at least 80%, such as at least 90%, for example at least 95% sequence identity herewith.

In one embodiment, the yeast strain of the invention carries one or more mutation(s) resulting in a mutant PAD gene encoding a mutant PAD1. For example, the yeast strain may carry a mutation in the PAD gene leading to a loss of PAD function, and in particular to a total loss of PAD function.

The yeast strain carrying one or more mutation(s) in the PAD gene leading to a loss of PAD function may carry different types of mutations, e.g. any of the mutations described herein in this section.

In one embodiment, the yeast strain of the invention carries one or more mutation(s) resulting in a mutant PAD gene encoding a mutant PAD protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the amino acid substitutions are located in the N-terminal region of PAD. In another embodiment, the amino acid substitutions are located in the C-terminal region of PAD.

In one embodiment, the yeast strain according to the invention carries a mutation in the PAD gene, wherein the mutation is:
a mutation resulting in a frameshift mutation;
a mutation resulting in formation of a premature stop codon in the PAD gene;
a mutation in a splice site of the PAD gene;
a mutation in the promoter region of the PAD gene; and/or
a mutation in an intron of the PAD gene

In another embodiment, wherein the yeast strain is a Dekkera anomalus yeast strain, said yeast strain carries a mutation in the DaPAD1 gene of SEQ ID NO:1, wherein the mutation is:
a mutation resulting in a frameshift mutation;
a mutation resulting in formation of a premature stop codon in the DaPAD1 gene;
a mutation in a splice site of the DaPAD1 gene;
a mutation in the promoter region of the DaPAD1 gene; and/or
a mutation in the an intron of the DaPAD1 gene.

In yet another embodiment, wherein the yeast strain is a Dekkera bruxellensis yeast strain, said yeast strain carries a mutation in the DbPAD2 gene of SEQ ID NO:5, wherein the mutation is:
a mutation resulting in a frameshift mutation;
a mutation resulting in formation of a premature stop codon in the DbPAD2 gene;
a mutation in a splice site of the DbPAD2 gene;
a mutation in the promoter region of the DbPAD2 gene; and/or
a mutation in the an intron of the DbPAD2 gene.

A mutation in the splice site, promoter region and/or an intron of the PAD gene may lead to aberrant splicing of PAD mRNA, and/or aberrant transcription of PAD mRNA and/or aberrant translation of PAD protein. Such yeast strain may in particular have reduced PAD mRNA levels as described herein below in this section and/or reduced PAD protein levels as described herein below in this section.

Loss of PAD function may be determined by any method known by a person skilled in the art. One way of determining PAD function, can be to determine the expression level of PAD either on the mRNA level or on the protein level.

In one embodiment, a yeast strain is considered to have a loss of PAD function when the yeast strain comprises less than 50%, preferably less than 25%, and even more preferably less than 10% mutant or wild type PAD mRNA compared to the level of PAD mRNA in a yeast strain comprising a wild type PAD gene, but otherwise of the same genotype. A yeast strain may be considered to have a total loss of PAD function when the yeast strain comprises less than 5%, preferably less than 1% mutant or wild type PAD mRNA compared to yeast strain comprising a wild type PAD gene, but otherwise of the same genotype. Said mutant PAD is mRNA encoded by a mutated PAD gene carrying a mutation in the mRNA coding region. In one embodiment, wherein said yeast strain is a Dekkera anomalus yeast strain, said PAD mRNA is DaPAD1 mRNA encoding a polypeptide of SEQ ID NO:2 or a functional homologue thereof, and a wild type DaPAD1 gene is a gene encoding the polypeptide of SEQ ID NO:2 or a functional homologue thereof. Said functional homologue preferably shares at least 80%, such as at least 90%, for example at least 95% sequence identity with SEQ ID NO:2. In one embodiment, a yeast strain with total loss of DaPAD1 function may contain no detectable mutant or wild type DaPAD1 mRNA, when determined by conventional quantitative RT-PCR. In another embodiment, wherein said yeast strain is a Dekkera bruxellensis yeast strain, said PAD mRNA is DbPAD2 mRNA encoding a polypeptide of SEQ ID NO:6 or a functional homologue thereof, and a wild type DbPAD2 gene is a gene encoding the polypeptide of SEQ ID NO:6 or a functional homologue thereof. Said functional homologue preferably shares at least 80%, such as at least 90%, for example at least 95% sequence identity with SEQ ID NO:6. In one embodiment, a yeast strain with total loss of DbPAD2 function may contain no detectable mutant or wild type DbPAD2 mRNA, when determined by conventional quantitative RT-PCR.

In one embodiment, a yeast strain is considered to have a loss of PAD function when the yeast strain comprises less than 50%, preferably less than 25%, and even more preferably less than 10% mutant or wild type PAD protein compared to the level of PAD protein in a yeast strain comprising a wild type PADgene, but otherwise of the same genotype. A yeast strain may be considered to have a total loss of PAD function when the yeast strain comprises less than 5%, preferably less than 1% mutant or wild type PAD protein compared to a yeast strain comprising a wild type PAD gene, but otherwise of the same genotype. Said mutant PAD protein is a polypeptide encoded by a mutated DPAD gene carrying a mutation in the coding region. In one embodiment, wherein said yeast strain is a Dekkera anomalus yeast strain, said PAD protein is a DaPAD1 polypeptide of SEQ ID NO:2 or a functional homologue thereof, and a wild type DaPAD1 gene is a gene encoding the polypeptide of SEQ ID NO:2 or a functional homologue thereof. Said functional homologue preferably shares at least 80%, such as at least 90%, for example at least 95% sequence identity with SEQ ID NO:2. In one embodiment, a yeast strain with total loss of DaPAD1 function may contain no detectable mutant or wild type DaPAD1 protein as detected by conventional Western blotting. In another embodiment, wherein said yeast strain is a Dekkera bruxellensis yeast strain, said PAD protein is a DbPAD2 polypeptide of SEQ ID NO:6 or a functional homologue thereof, and a wild type DbPAD2 gene is a gene encoding the polypeptide of SEQ ID NO:6 or a functional homologue thereof. Said functional homologue preferably shares at least 80%, such as at least 90%, for example at least 95% sequence identity with SEQ ID NO:6. In one embodiment, a yeast strain with total loss of DbPAD2 function may contain no detectable mutant or wild type DbPAD2 protein as detected by conventional Western blotting.

The yeast strain may for example have genotype I described herein above in embodiments of the invention, where the yeast strain is not capable converting more than 25% of p-coumaric acid into 4-ethylphenol. In other embodiments of the present invention, said yeast strain is not capable of converting more than 25% of ferulic acid into 4-ethylguaiacol.

### Genotype II: SOD1

The Dekkera yeast strain according to the invention may have the genotype II, wherein the genotype II is the presence of one or more mutations in or a deletion of the gene encoding SOD.

In embodiments of the invention, wherein the Dekkera yeast strain according to the invention has the genotype II, said Dekkera yeast strain in general also has characteristic I and/or II, preferably said yeast strain has both of characteristics I and II.

In one embodiment, wherein the yeast strain is a Dekkera anomalus yeast strain, said yeast strain has the genotype II, wherein genotype II comprises a mutation in or a deletion of the gene encoding DaSOD of SEQ ID NO:4 or a functional homologue thereof having at least 80% sequence identity herewith.

In another embodiment, wherein the yeast strain is a Dekkera bruxellensis yeast strain, said yeast strain has the genotype II, wherein genotype II comprises a mutation in or a deletion of the gene encoding DbSOD of SEQ ID NO:8 or a functional homologue thereof having at least 80%, such as at least 90%, for example at least 95% sequence identity herewith.

SOD may be responsible for the second reduction step of 4-vinylphenol into 4-ethylphenol, as well as the reduction of 4-vinylguaiacol into 4-ethylguaiacol.

In one embodiment of the present invention, the yeast strain according to the invention lacks the gene encoding SOD. Thus, the yeast strain may have a deletion of the gene encoding SOD.

In another embodiment, wherein the yeast strain is a Dekkera anomalus yeast strain, said yeast strain according to the invention lacks the gene encoding DaSOD of SEQ ID NO:4 or a functional homologue thereof having at least 80% sequence identity herewith. In other words, the yeast strain of the species Dekkera anomalus may have a deletion of the gene encoding DaSOD of SEQ ID NO:4 or a functional homologue thereof having at least 80% sequence identity herewith.

In yet another embodiment, wherein the yeast strain is a Dekkera bruxellensis yeast strain, said yeast strain according to the invention lacks the gene encoding DbSOD of SEQ ID NO:8 or a functional homologue thereof having at least 80%, such as at least 90%, for example at least 95% sequence identity herewith. In other words, the yeast strain of the species Dekkera bruxellensis may have a deletion of the gene encoding DbSOD of SEQ ID NO:8 or a functional homologue thereof having at least 80% sequence identity herewith.

In one embodiment, the yeast strain according to the present invention comprises one or more deletions in the gene encoding SOD so that said gene encodes mutant SOD lacking at least some of SOD, such as lacking at least 10% of SOD, such as lacking at least 20%, such as lacking at least 30%, such as lacking at least 40%, such as lacking at least 50%, such as lacking at least 60%, such as lacking at least 70%, such as lacking at least 80%, such as lacking at least 90% of SOD.

In another embodiment, wherein the yeast strain is a Dekkera anomalus yeast strain, said yeast strain lacks a portion of the gene encoding DaSOD, such as lacking at least 10% of DaSOD, such as lacking at least 20%, such as lacking at least 30%, such as lacking at least 40%, such as lacking at least 50%, such as lacking at least 60%, such as lacking at least 70%, such as lacking at least 80%, such as lacking at least 90% of DaSOD of SEQ ID NO:4 or a functional homologue thereof having at least 80% sequence identity herewith.

In yet another embodiment, wherein the yeast strain is a Dekkera bruxellensis yeast strain, said yeast strain lacks a portion of the gene encoding DbSOD, such as lacking at least 10% of DbSOD, such as lacking at least 20%, such as lacking at least 30%, such as lacking at least 40%, such as lacking at least 50%, such as lacking at least 60%, such as lacking at least 70%, such as lacking at least 80%, such as lacking at least 90% of DbSOD of SEQ ID NO:8 or a functional homologue thereof having at least 80% sequence identity herewith.

In one embodiment, the yeast strain of the invention carries one ore more mutation(s) resulting in a mutant SOD gene encoding a mutant SOD. For example the yeast strain carries a mutation in the SOD gene leading to a loss of SOD function, and in particular to a total loss of SOD function.

The yeast strain carrying one or more mutation(s) in the SOD gene leading to a loss of SOD function may carry different types of mutations, e.g. any of the mutations described herein in this section.

In one embodiment, the yeast strain of the invention carries one or more mutation(s) resulting in a mutant SOD gene encoding a mutant SOD protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the amino acid substitutions are located in the N-terminal region of SOD. In another embodiment, the amino acid substitutions are located in the C-terminal region of SOD.

In one embodiment, the yeast strain according to the invention carries a mutation in the SOD gene, wherein the mutation is:
a mutation resulting in a frameshift mutation;
a mutation resulting in formation of a premature stop codon in the SOD gene;
a mutation in a splice site of the SOD gene;
a mutation in the promoter region of the SOD gene; and/or
a mutation in an intron of the SOD gene.

In another embodiment, wherein the yeast strain is a Dekkera anomalus yeast strain, said yeast strain carries a mutation in the DaSOD gene of SEQ ID NO:3, wherein the mutation is:
a mutation resulting in a frameshift mutation;
a mutation resulting in formation of a premature stop codon in the DaSOD gene;
a mutation in a splice site of the DaSOD gene;
a mutation in the promoter region of the DaSOD gene; and/or
a mutation in the an intron of the DaSOD gene.

In yet another embodiment, wherein the yeast strain is a Dekkera bruxellensis yeast strain, said yeast strain carries a mutation in the DbSOD gene of SEQ ID NO:7, wherein the mutation is:
a mutation resulting in a frameshift mutation;
a mutation resulting in formation of a premature stop codon in the DbSOD gene;
a mutation in a splice site of the DbSOD gene;
a mutation in the promoter region of the DbSOD gene; and/or
a mutation in the an intron of the DbSOD gene.

A mutation in the splice site, promoter region and/or an intron of the SOD gene may lead to aberrant splicing of SOD mRNA, and/or aberrant transcription of SOD mRNA and/or aberrant translation of SOD protein. Such yeast strain may in particular have reduced SOD mRNA levels as described herein below in this section and/or reduced SOD protein levels as described herein below in this section.

Loss of SOD function may be determined by any method known by a person skilled in the art. One way of determining SOD function, can be to determine the expression level of SOD either on the mRNA level or on the protein level.

In one embodiment, a yeast strain is considered to have a loss of SOD function when the yeast strain comprises less than 50%, preferably less than 25%, and even more preferably less than 10% mutant or wild type SOD mRNA compared to the level of SOD mRNA in a yeast strain comprising a wild type SOD gene, but otherwise of the same genotype. A yeast strain may be considered to have a total loss of SOD function when the yeast strain comprises less than 5%, preferably less than 1% mutant or wild type SOD mRNA compared to yeast strain comprising a wild type SOD gene, but otherwise of the same genotype. Said mutant SOD is mRNA encoded by a mutated SOD gene carrying a mutation in the mRNA coding region. In one embodiment, wherein said yeast strain is a Dekkera anomalus yeast strain, said DaSOD mRNA is RNA encoding a polypeptide of SEQ ID NO:4 or a functional homologue thereof, and a wild type DaSOD gene is a gene encoding the polypeptide of SEQ ID NO:4 or a functional homologue thereof. Said functional homologue preferably shares at least 80% sequence identity with SEQ ID NO:4. In one embodiment, a yeast strain with total loss of DaSOD function may contain no detectable mutant or wild type DaSOD mRNA, when determined by conventional quantitative RT-PCR. In another embodiment, wherein said yeast strain is a Dekkera bruxellensis yeast strain, said DbSOD mRNA is RNA encoding a polypeptide of SEQ ID NO:8 or a functional homologue thereof, and a wild type DbSOD gene is a gene encoding the polypeptide of SEQ ID NO:8 or a functional homologue thereof. Said functional homologue preferably shares at least 80% sequence identity with SEQ ID NO:8. In one embodiment, a yeast strain with total loss of DbSOD function may contain no detectable mutant or wild type DbSOD mRNA, when determined by conventional quantitative RT-PCR.

In one embodiment, a yeast strain is considered to have a loss of SOD function when the yeast strain comprises less than 50%, preferably less than 25%, and even more preferably less than 10% mutant or wild type SOD protein compared to the level of SOD protein in a yeast strain comprising a wild type SOD gene, but otherwise of the same genotype. A yeast strain may be considered to have a total loss of SOD function when the yeast strain comprises less than 5%, preferably less than 1% mutant or wild type SOD protein compared to a yeast strain comprising a wild type SOD gene, but otherwise of the same genotype. Said mutant SOD protein is a polypeptide encoded by a mutated SOD gene carrying a mutation in the coding region. In one embodiment, wherein said yeast strain is a Dekkera anomalus yeast strain, said DaSOD protein is a polypeptide of SEQ ID NO:4 or a functional homologue thereof, and a wild type DaSOD gene is a gene encoding the polypeptide of SEQ ID NO:4 or a functional homologue thereof. Said functional homologue preferably shares at least 80% sequence identity with SEQ ID NO:4. In one embodiment, a yeast strain with total loss of DaSOD function may contain no detectable mutant or wild type DaSOD protein as detected by conventional Western blotting. In another embodiment, wherein said yeast strain is a Dekkera bruxellensis yeast strain, said DbSOD protein is a polypeptide of SEQ ID NO:8 or a functional homologue thereof, and a wild type DbSOD gene is a gene encoding the polypeptide of SEQ ID NO:8 or a functional homologue thereof. Said functional homologue preferably shares at least 80% sequence identity with SEQ ID NO:8. In one embodiment, a yeast strain with total loss of DbSOD function may contain no detectable mutant or wild type DbSOD protein as detected by conventional Western blotting.

The yeast strain may for example have genotype II described herein above in embodiments of the invention, where the yeast strain is not capable converting more than 25% of p-coumaric acid into 4-ethylphenol. In other embodiments of the present invention, said yeast strain is not capable of converting more than 25% of the ferulic acid into 4-ethylguaiacol.

### Genotype III: MTRA1

The Dekkera yeast strain according to the invention may have an additional genotype, genotype III, wherein the genotype III is the presence of one or more mutations in or a deletion of the gene encoding MTRA1.

In embodiments of the invention, wherein the Dekkera yeast strain according to the invention has the genotype III, said Dekkera yeast strain in general also has characteristic III.

The putative function of MTRA1 is predicted to be a high-affinity maltose transporter.

In one embodiment of the present invention, the yeast strain according to the invention lacks the gene encoding MTRA1. Thus, the yeast strain may have a deletion of the gene encoding MTRA1.

In another embodiment, wherein the yeast strain is a Dekkera anomalus yeast strain, said yeast strain according to the invention lacks the gene encoding DaMTRA1 of SEQ ID NO:10 or a functional homologue thereof having at least 98% sequence identity herewith. In other words, the yeast may have a deletion of the gene encoding DaMRTA1 of SEQ ID NO:10 or a functional homologue thereof having at least 98% sequence identity herewith.

In yet another embodiment, wherein the yeast strain is a Dekkera bruxellensis yeast strain, said yeast strain according to the invention lacks the gene encoding DbMTRA1 of SEQ ID NO:16 or a functional homologue thereof having at least 98% sequence identity herewith. In other words, the yeast may have a deletion of the gene encoding DbMRTA1 of SEQ ID NO:16 or a functional homologue thereof having at least 98% sequence identity herewith.

In one embodiment, the yeast strain according to the present invention comprises one or more deletions in the gene encoding MTRA1 so that said gene encodes mutant MTRA1 lacking at least some of MTRA1, such as lacking at least 10% of MTRA1, such as lacking at least 20%, such as lacking at least 30%, such as lacking at least 40%, such as lacking at least 50%, such as lacking at least 60%, such as lacking at least 70%, such as lacking at least 80%, such as lacking at least 90% of MTRA1.

In another embodiment, wherein the yeast strain is a Dekkera anomalus yeast strain, said yeast strain comprises a deletion in the gene encoding DaMTRA1 so that said gene encodes mutant DaMTRA1 lacking at least some of DaMTRA1, such as lacking at least 10% of DaMTRA1, such as lacking at least 20%, such as lacking at least 30%, such as lacking at least 40%, such as lacking at least 50%, such as lacking at least 60%, such as lacking at least 70%, such as lacking at least 80%, such as lacking at least 90% of DaMTRA1 of SEQ ID NO:10 or a functional homologue thereof having at least 98% sequence identity herewith.

In another embodiment, wherein the yeast strain is a Dekkera bruxellensis yeast strain, said yeast strain comprises a deletion in the gene encoding DbMTRA1 so that said gene encodes mutant DbMTRA1 lacking at least some of DbMTRA1, such as lacking at least 10% of DbMTRA1, such as lacking at least 20%, such as lacking at least 30%, such as lacking at least 40%, such as lacking at least 50%, such as lacking at least 60%, such as lacking at least 70%, such as lacking at least 80%, such as lacking at least 90% of DbMTRA1 of SEQ ID NO:16 or a functional homologue thereof having at least 98% sequence identity herewith.

In one embodiment, the yeast strain of the invention carries one or more mutation(s) resulting in a mutant MTRA1 gene encoding a mutant MTRA1. For example, the yeast strain may carry a mutation in the MTRA1 gene leading to a loss of MTRA1 function, and in particular to a total loss of MTRA1 function.

The yeast strain carrying one or more mutation(s) in the MTRA1 gene leading to a loss of MTRA1 function may carry different types of mutations, e.g. any of the mutations described herein in this section.

In one embodiment, the yeast strain of the invention carries one or more mutation(s) resulting in a mutant MTRA1 gene encoding a mutant MTRA1 protein comprising one or more amino acid substitutions, such as 4 or more, such as 8 or more, such as 12 or more, such as 14 or more amino acids substitutions. Said amino acid substitutions may be any amino acid substitutions, wherein the amino acid is replaced with another amino acid.

Preferably, the amino acid substitutions are located in the N-terminal region of the MTRA1.

Thus, in one embodiment, the yeast strain carries one or more mutation(s) resulting in a mutant MTRA1 gene encoding a mutant MTRA1 protein comprising one or more amino acid substitution, such as 4 or more, such as 8 or more, such as 12 or more, such as 14 or more amino acid substitutions in the N-terminal region consisting of amino acids 1 to 65 of MTRA1.

In another embodiment, wherein the yeast strain is a Dekkera anomalus yeast strain, said yeast strain carries one or more mutation(s) resulting in a mutant DaMTRA1 gene encoding a mutant DaMTRA1 protein comprising one or more amino acid substitution, such as 4 or more, such as 8 or more, such as 12 or more, such as 14 or more amino acid substitutions in the N-terminal region consisting of amino acids 1 to 65 of DaMTRA1 of SEQ ID NO: 10 or a functional homologue thereof having at least 98% sequence identity herewith.

In yet another embodiment, wherein the yeast strain is a Dekkera bruxellensis yeast strain, said yeast strain carries one or more mutation(s) resulting in a mutant DbMTRA1 gene encoding a mutant DbMTRA1 protein comprising one or more amino acid substitution, such as 4 or more, such as 8 or more, such as 12 or more, such as 14 or more amino acid substitutions in the N-terminal region consisting of amino acids 1 to 65 of DbMTRA1 of SEQ ID NO:16 or a functional homologue thereof having at least 98% sequence identity herewith.

In one embodiment, the yeast strain carries one or more mutation(s) resulting in a mutant MTRA1 gene encoding a mutant MTRA1 protein lacking one or more amino acid, such as lacking at least 4 amino acids, such as lacking at least 8, such as lacking at least 12, such as lacking at least 14 amino acids. In particular, said mutant MTRA1 protein may lack one or more of amino acids 1 to 65, such as lacking at least 4 amino acids, such as lacking at least 8, such as lacking at least 12, such as lacking at least 14 amino acids of amino acids in the N-terminal region consisting of amino acids 1 to 65 of MTRA1.

In another embodiment, wherein the yeast strain is a Dekkera anomalus yeast strain, said yeast strain carries a mutation resulting in a mutant DaMTRA1 gene encoding a mutant DaMTRA1 protein lacking one or more amino acid, such as lacking at least 4 amino acids, such as lacking at least 8, such as lacking at least 12, such as lacking at least 14 amino acids of SEQ ID NO:10 or a functional homologue thereof having at least 98% sequence identity herewith. In particular, said mutant DaMTRA1 protein may lack one or more of amino acids 1 to 65 of SEQ ID NO:10, such as lacking at least 4 amino acids, such as lacking at least 8, such as lacking at least 12, such as lacking at least 14 amino acids of amino acids 1 to 65 of SEQ ID NO:10, or a functional homologue thereof having at least 89% sequence identity herewith.

In yet another embodiment, wherein the yeast strain is a Dekkera bruxellensis yeast strain, said yeast strain carries a mutation resulting in a mutant DbMTRA1 gene encoding a mutant DbMTRA1 protein lacking one or more amino acid, such as lacking at least 4 amino acids, such as lacking at least 8, such as lacking at least 12, such as lacking at least 14 amino acids of SEQ ID NO:16 or a functional homologue thereof having at least 98% sequence identity herewith. In particular, said mutant DbMTRA1 protein may lack one or more of amino acids 1 to 65 of SEQ ID NO:16, such as lacking at least 4 amino acids, such as lacking at least 8, such as lacking at least 12, such as lacking at least 14 amino acids of amino acids 1 to 65 of SEQ ID NO:16, or a functional homologue thereof having at least 89% sequence identity herewith.

In one embodiment, the yeast strain of the invention carries one or more mutation(s) resulting in a mutant MTRA1 gene encoding a mutant MTRA1 protein lacking at least the 10 most N-terminal amino acids, for example at least the 20 most N-terminal amino acids, such as at least the 30 most N-terminal amino acids, for example at least the 60 most N-terminal amino acids MTRA1.

In another embodiment, wherein the yeast strain is a Dekkera anomalus yeast strain, said yeast strain of the invention carries one or more mutation(s) resulting in a mutant DaMTRA1 gene encoding a mutant DaMTRA1 protein lacking at least the 10 most N-terminal amino acids, for example at least the 20 most N-terminal amino acids, such as at least the 30 most N-terminal amino acids, for example at least the 60 most N-terminal amino acids of SEQ ID NO:10, or a functional homologue thereof having at least 98% sequence identity herewith. For example, the yeast strain may comprise a mutant DaMTRA1 gene encoding a mutant DaMTRA1 protein lacking at least the 64 most N-terminal amino acids of SEQ ID NO:10 or a functional homologue thereof having at least 98% sequence identity herewith.

In yet another embodiment, wherein the yeast strain is a Dekkera bruxellensis yeast strain, said yeast strain of the invention carries one or more mutation(s) resulting in a mutant DbMTRA1 gene encoding a mutant DbMTRA1 protein lacking at least the 10 most N-terminal amino acids, for example at least the 20 most N-terminal amino acids, such as at least the 30 most N-terminal amino acids, for example at least the 60 most N-terminal amino acids of SEQ ID NO:16, or a functional homologue thereof having at least 98% sequence identity herewith. For example, the yeast strain may comprise a mutant DbMTRA1 gene encoding a mutant DbMTRA1 protein lacking at least the 64 most N-terminal amino acids of SEQ ID NO:16 or a functional homologue thereof having at least 98% sequence identity herewith.

In one embodiment, wherein the yeast strain is a Dekkera anomalus yeast strain, said yeast strain of the invention carries a mutation resulting in a mutant DaMTRA1 gene encoding a truncated DaMTRA1 protein comprising an C-terminal fragment of DaMTRA1 comprising at the most the 579 C-terminal amino acids of SEQ ID NO:10 or a functional homologue thereof having at least 98% sequence identity herewith, for example at the most the 569 C-terminal amino acids of SEQ ID NO:10, such as at the most the 559 C-terminal amino acids of SEQ ID NO:10, such as at the most the 529 C-terminal amino acids of SEQ ID NO:10, preferably at the most the 524 C-terminal amino acids of SEQ ID NO:10.

In another embodiment, wherein the yeast strain is a Dekkera bruxellensis yeast strain, said yeast strain of the invention carries a mutation resulting in a mutant DbMTRA1 gene encoding a truncated DbMTRA1 protein comprising an C-terminal fragment of DbMTRA1 comprising at the most the 579 C-terminal amino acids of SEQ ID NO:16 or a functional homologue thereof having at least 98% sequence identity herewith, for example at the most the 569 C-terminal amino acids of SEQ ID NO:16, such as at the most the 559 C-terminal amino acids of SEQ ID NO:16, such as at the most the 529 C-terminal amino acids of SEQ ID NO:16, preferably at the most the 524 C-terminal amino acids of SEQ ID NO:16.

In one embodiment, wherein said yeast strain is a Dekkera anomalus yeast strain, said yeast strain is considered to have a loss of DaMTRA1 function if said yeast carries a mutation resulting in a DaMTRA1 gene encoding a mutant DaMTRA1 protein lacking one or more of the following regions:
W72-L155 of SEQ ID NO:10
F156-G382 of SEQ ID NO:10
A383-F532 of SEQ ID NO:10

In another embodiment, wherein the yeast strain is a Dekkera bruxellensis yeast strain, said yeast strain is considered to have a loss of DbMTRA1 function if said yeast carries a mutation resulting in a DbMTRA1 gene encoding a mutant DbMTRA1 protein lacking one or more of the following regions:
W72-M155 of SEQ ID NO:16
F156-V382 of SEQ ID NO:16
C383-F533 of SEQ ID NO:16

In one embodiment, the yeast strain according to the invention carries a mutation in the MTRA1 gene, wherein the mutation is:
a mutation resulting in a frameshift mutation;
a mutation resulting in formation of a premature stop codon in the MTRA1 gene;
a mutation in a splice site of the MTRA1 gene;
a mutation in the promoter region of the MTRA1 gene;
a mutation in the an intron of the MTRA1 gene.

In another embodiment, wherein the yeast strain is a Dekkera anomalus yeast strain, said yeast strain carries a mutation in the DaMTRA1 gene of SEQ ID NO:10, wherein the mutation is:
a mutation resulting in a frameshift mutation;
a mutation resulting in formation of a premature stop codon in the DaMTRA1 gene;
a mutation in a splice site of the DaMTRA1 gene;
a mutation in the promoter region of the DaMTRA1 gene; and/or
a mutation in the an intron of the DaMTRA1 gene.

In yet another embodiment, wherein the yeast strain is a Dekkera bruxellensis yeast strain, said yeast strain carries a mutation in the DaMTRA1 gene of SEQ ID NO:16, wherein the mutation is:
a mutation resulting in a frameshift mutation;
a mutation resulting in formation of a premature stop codon in the DbMTRA1 gene;
a mutation in a splice site of the DbMTRA1 gene;
a mutation in the promoter region of the DbMTRA1 gene; and/or
a mutation in the an intron of the DbMTRA1 gene.

A mutation in a splice site, a frameshift mutation or a mutation resulting in formation of a premature stop codon in general leads to a mutant gene encoding a truncated form of MTRA1. In one embodiment of the invention, wherein the yeast strain is a Dekkera anomalus yeast strain, said truncated DaMTRA1 may comprise an N-terminal fragment of DaMTRA1 comprising at the most the 500 N-terminal amino acids of SEQ ID NO:10, for example at the most the 400 N-terminal amino acids of SEQ ID NO:10, such as at the most the 300 N-terminal amino acids of SEQ ID NO:10, such as at the most the 200 N-terminal amino acids of SEQ ID NO:10, preferably at the most the 100 N-terminal amino acids of SEQ ID NO:10 or a functional homologue thereof having at least 98% sequence identity herewith. In another embodiment of the invention, wherein the yeast strain is a Dekkera bruxellensis yeast strain, said truncated DbMTRA1 may comprise an N-terminal fragment of DbMTRA1 comprising at the most the 500 N-terminal amino acids of SEQ ID NO:16, for example at the most the 400 N-terminal amino acids of SEQ ID NO:16, such as at the most the 300 N-terminal amino acids of SEQ ID NO:16, such as at the most the 200 N-terminal amino acids of SEQ ID NO:16, preferably at the most the 100 N-terminal amino acids of SEQ ID NO:16 or a functional homologue thereof having at least 98% sequence identity herewith.

A mutation in the splice site, promoter region and/or an intron of the MTRA1 gene may lead to aberrant splicing of MTRA1 mRNA, and/or aberrant transcription of MTRA1 mRNA and/or aberrant translation of MTRA1 protein. Such yeast strain may in particular have reduced MTRA1 mRNA levels as described herein below in this section and/or reduced MTRA1 protein levels as described herein below in this section.

Loss of MTRA1 function may be determined by determining by any method known by a person skilled in the art. One way of determining MTRA1 function, can be to determine the expression level of MTRA1 either on the mRNA level or on the protein level.

In one embodiment, a yeast strain is considered to have a loss of MTRA1 function when the yeast strain comprises less than 50%, preferably less than 25%, and even more preferably less than 10% mutant or wild type MTRA1 mRNA compared to the level of MTRA1 mRNA in a yeast strain comprising a wild type MTRA1 gene, but otherwise of the same genotype. A yeast strain may be considered to have a total loss of MTRA1 function when the yeast strain comprises less than 5%, preferably less than 1% mutant or wild type MTRA1 mRNA compared to yeast strain comprising a wild type MTRA1 gene, but otherwise of the same genotype. Said mutant MTRA1 is mRNA encoded by a mutated MTRA1 gene carrying a mutation in the mRNA coding region. In one embodiment, wherein said yeast strain is a Dekkera anomalus yeast strain, said DaMTRA1 mRNA is RNA encoding a polypeptide of SEQ ID NO:10 or a functional homologue thereof, and a wild type DaMTRA1 gene is a gene encoding the polypeptide of SEQ ID NO:10 or a functional homologue thereof. Said functional homologue preferably shares at least 98% sequence identity with SEQ ID NO:10. In one embodiment, a yeast strain with total loss of MTRA1 function may contain no detectable mutant or wild type MTRA1 mRNA, when determined by conventional quantitative RT-PCR. In another embodiment, wherein said yeast strain is a Dekkera bruxellensis yeast strain, said DbMTRA1 mRNA is RNA encoding a polypeptide of SEQ ID NO:16 or a functional homologue thereof, and a wild type DbMTRA1 gene is a gene encoding the polypeptide of SEQ ID NO:16 or a functional homologue thereof. Said functional homologue preferably shares at least 98% sequence identity with SEQ ID NO:16. In one embodiment, a yeast strain with total loss of MTRA1 function may contain no detectable mutant or wild type MTRA1 mRNA, when determined by conventional quantitative RT-PCR.

In one embodiment, a yeast strain is considered to have a loss of MTRA1 function when the yeast strain comprises less than 50%, preferably less than 25%, and even more preferably less than 10% mutant or wild type MTRA1 protein compared to the level of MTRA1 protein in a yeast strain comprising a wild type MTRA1 gene, but otherwise of the same genotype. A yeast strain may be considered to have a total loss of MTRA1 function when the yeast strain comprises less than 5%, preferably less than 1% mutant or wild type MTRA1 protein compared to a yeast strain comprising a wild type MTRA1 gene, but otherwise of the same genotype. Said mutant MTRA1 protein is a polypeptide encoded by a mutated MTRA1 gene carrying a mutation in the coding region. In one embodiment, wherein said yeast strain is a Dekkera anomalus yeast strain, said DaMTRA1 protein is a polypeptide of SEQ ID NO:10 or a functional homologue thereof, and a wild type DaMTRA1 gene is a gene encoding the polypeptide of SEQ ID NO:10 or a functional homologue thereof. Said functional homologue preferably shares at least 98% sequence identity with SEQ ID NO:10. In one embodiment, a yeast strain with total loss of DaMTRA1 function may contain no detectable mutant or wild type DaMTRA1 protein as detected by conventional Western blotting. In another embodiment, wherein said yeast strain is a Dekkera bruxellensis yeast strain, said DbMTRA1 protein is a polypeptide of SEQ ID NO:16 or a functional homologue thereof, and a wild type DbMTRA1 gene is a gene encoding the polypeptide of SEQ ID NO:16 or a functional homologue thereof. Said functional homologue preferably shares at least 98% sequence identity with SEQ ID NO:16. In one embodiment, a yeast strain with total loss of DbMTRA1 function may contain no detectable mutant or wild type DbMTRA1 protein as detected by conventional Western blotting.

The yeast strain may for example have genotype III in embodiments of the invention, where the yeast strain besides not being capable converting more than 25% of p-coumaric acid into 4-ethylphenol and/or not capable of converting more than 25% of ferulic acid into 4-ethylguaiacol, is not capable of utilizing more than 2% maltose.

### Genotype IV—ISOM and ISOM(2)

The Dekkera yeast strain according to the invention may have the genotype IV, wherein the genotype IV is the presence of one or more mutations in or a deletion of one or more of the genes encoding ISOM.

In embodiments of the invention, wherein the Dekkera yeast strain according to the invention has the genotype IV, said Dekkera yeast strain in general also has characteristic III.

This major isomaltase, ISOM, is potentially an enzyme with alpha-glucosidase activity capable of breaking down alpha linked di-saccharides such as maltose. The result of maltose break down is two monosaccharide molecules of glucose, which can then be fermented by the yeast. In one embodiment of the invention, wherein the yeast strain is a Dekkera anomalus yeast strain, the yeast strain carries one copy of the BaISOM gene and hence one BaISOM protein. In another embodiment of the invention, wherein the yeast strain is a Dekkera bruxellensis yeast strain, the yeast strain carries two copies of the potential isomaltases along the genome, herein denoted “ISOM(2)” and “ISOM(1)”. The two copies have different nucleotide sequences and amino acid sequences.

In one embodiment of the present invention, the yeast strain according to the invention lacks at least one gene encoding an ISOM protein. Thus, the yeast strain may have one or more deletion(s) of the gene(s) encoding ISOM.

In another embodiment, wherein the yeast strain is a Dekkera anomalus yeast strain, said yeast strain according to the invention lacks the entire DaISOM gene encoding DaISOM of SEQ ID NO:12 or a functional homologue thereof having at least 98% sequence identity herewith. In other words, the yeast may have a deletion of the gene encoding ISOM of SEQ ID NO:12 or a functional homologue thereof having at least 98% sequence identity herewith.

In yet another embodiment, wherein the yeast strain is a Dekkera bruxellensis yeast strain, said yeast strain according to the invention lacks the entire DbISOM(2) gene encoding DbISOM(2) of SEQ ID NO:18 or a functional homologue thereof having at least 98% sequence identity herewith. In other words, the yeast may have a deletion of the gene encoding DbISOM(2) of SEQ ID NO:18 or a functional homologue thereof having at least 98% sequence identity herewith.

In one embodiment, the yeast strain according to the invention comprises a deletion in one or more of the genes encoding ISOM so that said gene carrying a deletion encodes a mutant ISOM lacking at least 10% of ISOM, such as lacking at least 20%, such as lacking at least 30%, such as lacking at least 40%, such as lacking at least 50%, such as lacking at least 60%, such as lacking at least 70%, such as lacking at least 80%, such as lacking at least 90% of ISOM.

In another embodiment, wherein the yeast strain is a Dekkera anomalus yeast strain, said yeast strain comprises a deletion in the gene encoding DaISOM so that said gene encodes mutant DaISOM lacking at least 10% of DaISOM, such as lacking at least 20%, such as lacking at least 30%, such as lacking at least 40%, such as lacking at least 50%, such as lacking at least 60%, such as lacking at least 70%, such as lacking at least 80%, such as lacking at least 90% of DaISOM of SEQ ID NO:12 or a functional homologue thereof having at least 98% sequence identity herewith.

In another embodiment, wherein the yeast strain is a Dekkera bruxellensis yeast strain, said yeast strain comprises a deletion in the gene encoding DbISOM(2) and/or DbISOM(1) so that said gene encodes mutant DbISOM(2) and/or DbISOM(1) lacking at least 10% of DbISOM(2) and/or DbISOM(1), such as lacking at least 20%, such as lacking at least 30%, such as lacking at least 40%, such as lacking at least 50%, such as lacking at least 60%, such as lacking at least 70%, such as lacking at least 80%, such as lacking at least 90% of DbISOM(2) of SEQ ID NO:18 or a functional homologue thereof having at least 98% sequence identity herewith and/or DbISOM(1) of SEQ ID NO:22 or a functional homologue thereof having at least 98% sequence identity herewith.

In one embodiment, the yeast strain of the invention carries one or more mutation(s) resulting in one or more mutant ISOM genes encoding one or more mutant ISOM(s).

In one embodiment, wherein the yeast strain is a Dekkera bruxellensis yeast strain, it is preferred that the yeast strain carries a mutation in the ISOM(2) gene leading to a loss of ISOM(2) function, and in particular to a total loss of ISOM(2) function.

In another embodiment, wherein the yeast strain is a Dekkera anomalus yeast strain, it is preferred that the yeast strain carries a mutation in the ISOM gene leading to a loss of ISOM function, and in particular to a total loss of ISOM function.

The yeast strain carrying one or more mutation(s) in the one or more ISOM genes leading to a loss of function of one or more ISOM(s) may carry different types of mutations, e.g. any of the mutations described herein in this section.

In one embodiment, the yeast strain of the invention carries a frameshift mutation, and/or a mutation leading to a premature stop codon and/or a splice mutation in one or more ISOM genes resulting in a truncation of one or more of the ISOM proteins.

In one embodiment, wherein the yeast strain is a Dekkera anomalus yeast strain, said yeast strain carries a frameshift mutation, and/or a mutation leading to a premature stop codon and/or a splice mutation resulting in a mutant DaISOM gene encoding a mutant DaISOM protein lacking one or more amino acid, such as lacking at least 50 amino acids, such as lacking at least 100, such as lacking at least 150, such as lacking at least 200 amino acids of SEQ ID NO:12 or a functional homologue thereof having at least 98% sequence identity herewith.

In another embodiment, wherein the yeast strain is a Dekkera bruxellensis yeast strain, said yeast strain carries a frameshift mutation, and/or a mutation leading to a premature stop codon and/or a splice mutation resulting in a mutant DbISOM(2) gene encoding a mutant DbISOM(2) protein lacking one or more amino acid, such as lacking at least 50 amino acids, such as lacking at least 100, such as lacking at least 150, such as lacking at least 200 amino acids of SEQ ID NO:18 or a functional homologue thereof having at least 98% sequence identity herewith.

In one embodiment, wherein the yeast strain is a Dekkera anomalus yeast strain, said yeast strain of the invention carries a frameshift mutation, and/or a mutation leading to a premature stop codon and/or a splice mutation resulting in a mutant DaISOM gene encoding a mutant DaISOM protein lacking at least the 50 most C-terminal amino acids, for example lacking at least the 100 most C-terminal amino acids, such as at least the 150 most C-terminal amino acids, such as at least the 200 most C-terminal amino acids of SEQ ID NO:12. For example, the yeast strain may comprise a mutant DaISOM gene encoding a mutant DaISOM protein lacking at least the 237 most C-terminal amino acids of SEQ ID NO:12 or a functional homologue thereof having at least 98% sequence identity herewith.

In another embodiment, wherein the yeast strain is a Dekkera bruxellensis yeast strain, said yeast strain of the invention carries a frameshift mutation, and/or a mutation leading to a premature stop codon and/or a splice mutation resulting in a mutant DbISOM(2) gene encoding a mutant DbISOM(2) protein lacking at least the 50 most C-terminal amino acids, for example lacking at least the 100 most C-terminal amino acids, such as at least the 150 most C-terminal amino acids, such as at least the 200 most C-terminal amino acids of SEQ ID NO:18. For example, the yeast strain may comprise a mutant DbISOM(2) gene encoding a mutant DbISOM protein lacking at least the 237 most C-terminal amino acids of SEQ ID NO:18 or a functional homologue thereof having at least 98% sequence identity herewith.

In one embodiment, wherein the yeast strain is a Dekkera anomalus yeast strain, said yeast strain of the invention carries a frameshift mutation, and/or a mutation leading to a premature stop codon and/or a splice mutation resulting in a mutant DaISOM gene encoding a truncated DaISOM protein comprising an N-terminal fragment of DaISOM comprising at the most the 500 N-terminal amino acids of SEQ ID NO:12, for example at the most the 450 N-terminal amino acids, such as at the most the 400 N-terminal amino acids of SEQ ID NO:12, preferably at the most the 350 N-terminal amino acids of SEQ ID NO:12 or a functional homologue thereof having at least 80% sequence identity herewith.

In another embodiment, wherein the yeast strain is a Dekkera bruxellensis yeast strain, said yeast strain of the invention carries a frameshift mutation, and/or a mutation leading to a premature stop codon and/or a splice mutation resulting in a mutant DbISOM(2) gene encoding a truncated DbISOM(2) protein comprising an N-terminal fragment of DbISOM(2) comprising at the most the 500 N-terminal amino acids of SEQ ID NO:18, for example at the most the 450 N-terminal amino acids, such as at the most the 400 N-terminal amino acids of SEQ ID NO:18, preferably at the most the 350 N-terminal amino acids of SEQ ID NO:18 or a functional homologue thereof having at least 80% sequence identity herewith.

In one embodiment, the yeast strain of the invention carries a mutation resulting in a mutant ISOM gene encoding a mutant ISOM proteins, wherein the mutant ISOM comprises at least 50 amino acids substitutions, such as at least 100, such as at least 150, such as at least 200 amino acids substitutions compared to ISOM in a yeast strain comprising a wild type ISOM gene. Said amino acid substitutions may be any amino acid substitutions, wherein the amino acid is replaced with another amino acid.

In another embodiment, wherein the yeast strain is a Dekkera anomalus yeast strain, said yeast strain carries a mutation resulting in a mutant DaISOM gene encoding a mutant DaISOM protein, wherein the mutant DaISOM comprises at least 50 amino acids substitutions, such as at least 100, such as at least 150, such as at least 200 amino acids substitutions compared to DaISOM in a yeast strain comprising a wild type DaISOM gene. Said amino acid substitutions may be any amino acid substitutions, wherein the amino acid is replaced with another amino acid.

In yet another embodiment, wherein the yeast strain is a Dekkera bruxellensis yeast strain, said yeast strain carries a mutation resulting in a mutant DabISOM(2) gene encoding a mutant DbISOM(2) protein, wherein the mutant DbISOM(2) comprises at least 50 amino acids substitutions, such as at least 100, such as at least 150, such as at least 200 amino acids substitutions compared to DbISOM(2) in a yeast strain comprising a wild type DbISOM(2) gene. Said amino acid substitutions may be any amino acid substitutions, wherein the amino acid is replaced with another amino acid.

In one embodiment, the yeast strain according to the invention carries a mutation in one or more of the ISOM genes, wherein the mutation is:
a mutation resulting in a frameshift mutation;
a mutation resulting in one or more amino acid substitution in one or more ISOM(s);
a mutation resulting in formation of a premature stop codon in one or more ISOM genes;
a mutation in a splice site in one or more ISOM genes;
a mutation in the promoter region of one or more ISOM genes; and/or
a mutation in an intron of one or more ISOM genes.

In one embodiment, wherein the yeast strain is a Dekkera anomalus yeast strain, said yeast strain according to the invention carries a mutation in the DaISOM gene, wherein the mutation is:
a mutation resulting in a frameshift mutation;
a mutation resulting in one or more amino acid substitution of DaISOM;
a mutation resulting in formation of a premature stop codon in the DaISOM gene;
a mutation in a splice site of the DaISOM gene;
a mutation in the promoter region of the DaISOM gene; and/or
a mutation in the an intron of the DaISOM gene.

In one embodiment, wherein the yeast strain is a Dekkera bruxellensis yeast strain, said yeast strain according to the invention carries a mutation in the DbISOM(2) gene, wherein the mutation is:
a mutation resulting in a frameshift mutation;
a mutation resulting in one or more amino acid substitution of DbISOM(2);
a mutation resulting in formation of a premature stop codon in the DbISOM(2) gene;
a mutation in a splice site of the DbISOM(2) gene;
a mutation in the promoter region of the DaISOM(2) gene; and/or
a mutation in the an intron of the DbISOM(2) gene.

In a preferred embodiment, wherein the yeast strain is a Dekkera bruxellensis yeast strain, the mutation is a mutation resulting in a frameshift mutation.

A mutation in the splice site, promoter region and/or an intron of one or more ISOM genes may lead to aberrant splicing of ISOM mRNA, and/or aberrant transcription of ISOM mRNA and/or aberrant translation of ISOM protein. Such yeast strain may in particular have reduced ISOM mRNA levels as described herein below in this section and/or reduced ISOM protein levels as described herein below in this section.

Loss of ISOM function may be determined by determining by any method known by a person skilled in the art. One way of determining ISOM function, can be to determine the expression level of ISOM either on the mRNA level or on the protein level.

In one embodiment, a yeast strain is considered to have a loss of ISOM function when the yeast strain comprises less than 50%, preferably less than 25%, and even more preferably less than 10% mutant or wild type ISOM mRNA compared to the level of ISOM mRNA in a yeast strain comprising a wild type ISOM gene, but otherwise of the same genotype. A yeast strain may be considered to have a total loss of ISOM function when the yeast strain comprises less than 5%, preferably less than 1% mutant or wild type ISOM mRNA compared to yeast strain comprising a wild type ISOM gene, but otherwise of the same genotype. Said mutant ISOM is mRNA encoded by a mutated ISOM gene carrying a mutation in the mRNA coding region. In one embodiment, wherein said yeast strain is a Dekkera anomalus yeast strain, said DaISOM mRNA is RNA encoding a polypeptide of SEQ ID NO:12 or a functional homologue thereof, and a wild type DaISOM gene is a gene encoding the protein of SEQ ID NO:12 or a functional homologue thereof. Said functional homologue preferably shares at least 98% sequence identity with SEQ ID NO:12. In one embodiment, a yeast strain with total loss of DaISOM function may contain no detectable mutant or wild type DaISOM mRNA, when determined by conventional quantitative RT-PCR. In one embodiment, wherein said yeast strain is a Dekkera bruxellensis yeast strain, said DbISOM(2) mRNA or DbISOM(1) mRNA is RNA encoding a polypeptide of SEQ ID NO:18 or a functional homologue thereof or encoding a polypeptide of SEQ ID NO:22 or a functional homologue thereof, and a wild type DbISOM(2) gene or DbISOM(1) gene is a gene encoding the protein of SEQ ID NO:18 or a functional homologue thereof or the protein of SEQ ID NO:22 or a functional homologue thereof. Said functional homologue preferably shares at least 98% sequence identity with SEQ ID NO:18 or SEQ ID NO:22. In one embodiment, a yeast strain with total loss of DbISOM(2) or DbISOM(1) function may contain no detectable mutant or wild type DbISOM(2) mRNA or DbISOM(1) mRNA, when determined by conventional quantitative RT-PCR.

In one embodiment, a yeast strain is considered to have a loss of ISOM function when the yeast strain comprises less than 50%, preferably less than 25%, and even more preferably less than 10% mutant or wild type ISOM protein compared to the level of ISOM protein in a yeast strain comprising a wild type ISOM gene, but otherwise of the same genotype. A yeast strain may be considered to have a total loss of ISOM function when the yeast strain comprises less than 5%, preferably less than 1% mutant or wild type ISOM protein compared to a yeast strain comprising a wild type ISOM gene, but otherwise of the same genotype. Said mutant ISOM protein is a polypeptide encoded by a mutated ISOM gene carrying a mutation in the coding region. In one embodiment, wherein said yeast strain is a Dekkera anomalus yeast strain, said DaISOM(2) protein is a polypeptide of SEQ ID NO:12 or a functional homologue thereof, and a wild type DaISOM(2) gene is a gene encoding the protein of SEQ ID NO:12 or a functional homologue thereof. Said functional homologue preferably shares at least 98% sequence identity with SEQ ID NO:12. In one embodiment, a yeast strain with total loss of DaISOM function may contain no detectable mutant or wild type DaISOM protein as detected by conventional Western blotting. In another embodiment, wherein said yeast strain is a Dekkera bruxellensis yeast strain, said DbISOM(2) mRNA or DbISOM(1) mRNA is RNA encoding a polypeptide of SEQ ID NO:18 or a functional homologue thereof or encoding a polypeptide of SEQ ID NO:22 or a functional homologue thereof, and a wild type DbISOM(2) gene or DbISOM(1) gene is a gene encoding the protein of SEQ ID NO:18 or a functional homologue thereof or the protein of SEQ ID NO:22 or a functional homologue thereof. Said functional homologue preferably shares at least 98% sequence identity with SEQ ID NO:18 or SEQ ID NO:22. In one embodiment, a yeast strain with total loss of DbISOM(2) or DbISOM(1) function may contain no detectable mutant or wild type DbISOM(2) mRNA or DbISOM(1) protein as detected by conventional Western blotting.

The yeast strain may for example have genotype IV in embodiments of the invention, where the yeast strain besides not being capable converting more than 25% of p-coumaric acid into 4-ethylphenol and/or not capable of converting more than 25% of ferulic acid into 4-ethylguaiacol, is not capable of utilizing more than 2% maltose.

### Genotype V— MTRA2

The yeast strain according to the present invention may have the genotype V, wherein the genotype V is the presence of one or more mutations in or a deletion of the gene encoding MTRA2.

In embodiments of the invention, wherein the Dekkera yeast strain according to the invention has the genotype V, said Dekkera yeast strain in general also has characteristic III.

The putative function of MTRA2 is predicted to be a high-affinity maltose transporter.

In one embodiment of the present invention, the yeast strain according to the invention lacks the gene encoding MTRA1. Thus, the yeast strain may have a deletion of the gene encoding MTRA1.

In another embodiment, wherein the yeast strain is a Dekkera anomalus yeast strain, said yeast strain according to the invention lacks the entire DaMTRA2 gene encoding DaMTRA2 of SEQ ID NO:14 or a functional homologue thereof having at least 98% sequence identity herewith. In other words, the yeast strain of the species Dekkera anomalus may have a deletion of the gene encoding DaMTRA2 of SEQ ID NO:14 or a functional homologue thereof having at least 98% sequence identity herewith.

In yet another embodiment, wherein the yeast strain is a Dekkera bruxellensis yeast strain, said yeast strain according to the invention lacks the entire DbMTRA2 gene encoding DbMTRA2 of SEQ ID NO:20 or a functional homologue thereof having at least 98% sequence identity herewith. In other words, the yeast strain of the species Dekkera bruxellensis may have a deletion of the gene encoding DbMTRA2 of SEQ ID NO:20 or a functional homologue thereof having at least 98% sequence identity herewith.

In one embodiment, the yeast strain according to the present invention comprises one or more deletions in the gene encoding MTRA2 so that said gene encodes mutant MTRA2 lacking at least some of MTRA2, such as lacking at least 10% of MTRA2, such as lacking at least 20%, such as lacking at least 30%, such as lacking at least 40%, such as lacking at least 50%, such as lacking at least 60%, such as lacking at least 70%, such as lacking at least 80%, such as lacking at least 90% of MTRA2.

In another embodiment, wherein the yeast strain is a Dekkera anomalus yeast strain, said yeast strain lacks a portion of the DaMTRA2 gene hereby encoding only a part of the DaMTRA2, such as at the most 90% of DaMTRA2, such as at the most 80%, such as at the most 70%, such as at the most 60%, such as at the most 50%, such as at the most 40%, such as at the most 30%, such as at the most 30%, such as at the most 20% of DaMTRA2 of SEQ ID NO:14 or a functional homologue thereof having at least 98% sequence identity herewith.

In yet another embodiment, wherein the yeast strain is a Dekkera bruxellensis yeast strain, said yeast strain lacks a portion of the DbMTRA2 gene hereby encoding only a part of the DbMTRA2, such as at the most 90% of DbMTRA2, such as at the most 80%, such as at the most 70%, such as at the most 60%, such as at the most 50%, such as at the most 40%, such as at the most 30%, such as at the most 30%, such as at the most 20% of DbMTRA2 of SEQ ID NO:20 or a functional homologue thereof having at least 98% sequence identity herewith.

In one embodiment, the yeast strain of the invention carries one or more mutation(s) resulting in a mutant MTRA2 gene encoding a mutant MTRA2. For example, the yeast strain may carry a mutation in the MTRA2 gene leading to a loss of MTRA2 function, and in particular to a total loss of MTRA2 function.

The yeast strain carrying one or more mutation(s) in the MTRA2 gene leading to a loss of MTRA2 function may carry different types of mutations, e.g. any of the mutations described herein in this section.

In one embodiment, the yeast strain of the invention carries one or more mutation(s) resulting in a mutant MTRA2 gene encoding a mutant MTRA2 protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In another embodiment, wherein the yeast strain is a Dekkera anomalus yeast strain, said yeast strain carries a mutation resulting in a mutant DaMTRA2 gene encoding a mutant DaMTRA2 protein lacking one or more amino acid, such as lacking at least 5 amino acids, such as lacking at least 10, such as lacking at least 15, such as lacking at least 20 amino acids of SEQ ID NO:14 or a functional homologue thereof having at least 80% sequence identity herewith.

In yet another embodiment, wherein the yeast strain is a Dekkera bruxellensis yeast strain, said yeast strain carries a mutation resulting in a mutant DbMTRA2 gene encoding a mutant DbMTRA2 protein lacking one or more amino acid, such as lacking at least 5 amino acids, such as lacking at least 10, such as lacking at least 15, such as lacking at least 20 amino acids of SEQ ID NO:20 or a functional homologue thereof having at least 80% sequence identity herewith.

In one embodiment, the yeast strain of the invention carries a mutation resulting in a mutant MTRA2 gene encoding a mutant MTRA2 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids.

In another embodiment, wherein the yeast strain is a Dekkera anomalus yeast strain, said yeast strain carries a mutation resulting in a mutant DaMTRA2 gene encoding a mutant DaMTRA2 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:14 or a functional homologue thereof having at least 98% sequence identity herewith.

In yet another embodiment, wherein the yeast strain is a Dekkera bruxellensis yeast strain, said yeast strain carries a mutation resulting in a mutant DbMTRA2 gene encoding a mutant DbMTRA2 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:20 or a functional homologue thereof having at least 98% sequence identity herewith.

In one embodiment, the yeast strain of the invention carries a mutation resulting in a mutant MTRA2 gene encoding a mutant MTRA2 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids.

In another embodiment, wherein the yeast strain is a Dekkera anomalus yeast strain, said yeast strain of the invention carries a mutation resulting in a mutant DaMTRA2 gene encoding a mutant DaMTRA2 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:14 or a functional homologue thereof having at least 98% sequence identity herewith.

In yet another embodiment, wherein the yeast strain is a Dekkera bruxellensis yeast strain, said yeast strain of the invention carries a mutation resulting in a mutant DbMTRA2 gene encoding a mutant DbMTRA2 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:20 or a functional homologue thereof having at least 98% sequence identity herewith.

In one embodiment, the yeast strain of the invention carries a mutation resulting in a frame shift mutation of the MTRA2 gene.

In one embodiment, the yeast strain of the invention carries a mutation resulting in formation of a premature stop codon in the MTRA2 gene.

In another embodiment, the mutation is a mutation in a splice site of the MTRA2 gene. Said mutation may lead to aberrant splicing of MTRA2 mRNA.

In one embodiment, the yeast strain carries a mutation in the promoter region of the MTRA2 gene or in an intron of the MTRA2 gene leading to aberrant transcription of MTRA2 mRNA and/or aberrant translation of MTRA2 protein. Such yeast strain may in particular have reduced MTRA2 mRNA levels as described herein below in this section and/or reduced MTRA2 protein levels as described herein below in this section.

Loss of MTRA2 function may be determined by determining by any method known by a person skilled in the art. One way of determining MTRA2 function, can be to determine the expression level of MTRA2 either on the mRNA level or on the protein level.

In one embodiment, a yeast strain is considered to have a loss of MTRA2 function when the yeast strain comprises less than 50%, preferably less than 25%, and even more preferably less than 10% mutant or wild type MTRA2 mRNA compared to the level of MTRA2 mRNA in a yeast strain comprising a wild type MTRA2 gene, but otherwise of the same genotype. A yeast strain may be considered to have a total loss of MTRA2 function when the yeast strain comprises less than 5%, preferably less than 1% mutant or wild type MTRA2 mRNA compared to yeast strain comprising a wild type MTRA2 gene, but otherwise of the same genotype. Said mutant MTRA2 is mRNA encoded by a mutated MTRA2 gene carrying a mutation in the mRNA coding region. In one embodiment, wherein said yeast strain is a Dekkera anomalus yeast strain, said DaMTRA2 mRNA is RNA encoding a polypeptide of SEQ ID NO:14 or a functional homologue thereof, and a wild type DaMTRA2 gene is a gene encoding the polypeptide of SEQ ID NO:14 or a functional homologue thereof. Said functional homologue preferably shares at least 98% sequence identity with SEQ ID NO:14. In one embodiment, a yeast strain with total loss of DaMTRA2 function may contain no detectable mutant or wild type DaMTRA2 mRNA, when determined by conventional quantitative RT-PCR. In another embodiment, wherein said yeast strain is a Dekkera bruxellensis yeast strain, said DbMTRA2 mRNA is RNA encoding a polypeptide of SEQ ID NO:20 or a functional homologue thereof, and a wild type DaMTRA2 gene is a gene encoding the polypeptide of SEQ ID NO:20 or a functional homologue thereof. Said functional homologue preferably shares at least 98% sequence identity with SEQ ID NO:20. In one embodiment, a yeast strain with total loss of DbMTRA2 function may contain no detectable mutant or wild type DbMTRA2 mRNA, when determined by conventional quantitative RT-PCR.

In one embodiment, a yeast strain is considered to have a loss of MTRA2 function when the yeast strain comprises less than 50%, preferably less than 25%, and even more preferably less than 10% mutant or wild type MTRA2 protein compared to the level of MTRA2 protein in a yeast strain comprising a wild type MTRA2 gene, but otherwise of the same genotype. A yeast strain may be considered to have a total loss of MTRA2 function when the yeast strain comprises less than 5%, preferably less than 1% mutant or wild type MTRA2 protein compared to a yeast strain comprising a wild type MTRA2 gene, but otherwise of the same genotype. Said mutant MTRA2 protein is a polypeptide encoded by a mutated MTRA2 gene carrying a mutation in the coding region. In one embodiment, wherein said yeast strain is a Dekkera anomalus yeast strain, said DaMTRA2 protein is a polypeptide of SEQ ID NO:14 or a functional homologue thereof, and a wild type DaMTRA2 gene is a gene encoding the polypeptide of SEQ ID NO:14 or a functional homologue thereof. Said functional homologue preferably shares at least 98% sequence identity with SEQ ID NO:14. In one embodiment, a yeast strain with total loss of DaMTRA2 function may contain no detectable mutant or wild type DaMTRA2 protein as detected by conventional Western blotting. In another embodiment, wherein said yeast strain is a Dekkera bruxellensis yeast strain, said DbMTRA2 protein is a polypeptide of SEQ ID NO:20 or a functional homologue thereof, and a wild type DbMTRA2 gene is a gene encoding the polypeptide of SEQ ID NO:20 or a functional homologue thereof. Said functional homologue preferably shares at least 98% sequence identity with SEQ ID NO:20. In one embodiment, a yeast strain with total loss of DbMTRA2 function may contain no detectable mutant or wild type DbMTRA2 protein as detected by conventional Western blotting.

The yeast strain may for example have genotype V in embodiments of the invention, where the yeast strain besides not being capable converting more than 25% of p-coumaric acid into 4-ethylphenol and/or not capable of converting more than 25% of ferulic acid into 4-ethylguaiacol, is not capable of utilizing more than 2% maltose.

### Genotype VI—MTRA3

The yeast strain according to the present invention may have the genotype VI, wherein the genotype VI is the presence of one or more mutations in or a deletion of the gene encoding MTRA3.

In embodiments of the invention, wherein the Dekkera yeast strain according to the invention has the genotype VI, said Dekkera yeast strain in general also have characteristic III.

The putative function of MTRA3 is predicted to be a maltose transporter.

In one embodiment, the yeast strain according to the invention lacks the entire DbMTRA3 gene encoding DbMTRA3 of SEQ ID NO:26 or a functional homologue thereof having at least 98% sequence identity herewith.

In another embodiment, the yeast strain lacks a portion of the DbMTRA3 gene hereby encoding only a part of the DbMTRA3, such as at the most 90% of DbMTRA3, such as at the most 80%, such as at the most 70%, such as at the most 60%, such as at the most 50%, such as at the most 40%, such as at the most 30%, such as at the most 30%, such as at the most 20% of DbMTRA3 of SEQ ID NO:26.

In one embodiment, the yeast strain of the invention carries a mutation resulting in a mutant MTRA3 gene encoding a mutant MTRA3. It is preferred that the yeast strain carries a mutation in the MTRA3 gene leading to a loss of MTRA3 function, and in particular to a total loss of MTRA3 function.

The yeast strain carrying a mutation in the MTRA3 gene leading to a loss of MTRA3 function may carry different types of mutations, e.g. any of the mutations described herein in this section.

In one embodiment, the yeast strain of the invention carries a mutation resulting in a mutant MTRA3 gene encoding a mutant MTRA3 protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the yeast strain carries a mutation resulting in a mutant DbMTRA3 gene encoding a mutant DbMTRA3 protein lacking one or more amino acid, such as lacking at least 5 amino acids, such as lacking at least 10, such as lacking at least 15, such as lacking at least 20 amino acids of SEQ ID NO:26.

In one embodiment, the yeast strain of the invention carries a mutation resulting in a mutant DbMTRA3 gene encoding a mutant DbMTRA3 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:26.

In another embodiment, the yeast strain of the invention carries a mutation resulting in a mutant DbMTRA3 gene encoding a mutant DbMTRA3 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:26.

In one embodiment, the yeast strain of the invention carries a mutation resulting in a frame shift mutation of the MTRA3 gene.

In one embodiment, the yeast strain of the invention carries a mutation resulting in formation of a premature stop codon in the MTRA3 gene.

In another embodiment, the mutation is a mutation in a splice site of the MTRA3 gene. Said mutation may lead to aberrant splicing of MTRA3 mRNA.

In one embodiment, the yeast strain carries a mutation in the promoter region of the MTRA3 gene or in an intron of the MTRA3 gene leading to aberrant transcription of MTRA3 mRNA and/or aberrant translation of MTRA3 protein. Such yeast strain may in particular have reduced MTRA3 mRNA levels as described herein below in this section and/or reduced MTRA3 protein levels as described herein below in this section.

Loss of MTRA3 function may be determined by determining by any method known by a person skilled in the art. One way of determining MTRA3 function, can be to determine the expression level of MTRA3 either on the mRNA level or on the protein level.

In one embodiment, a yeast strain is considered to have a loss of MTRA3 function when the yeast strain comprises less than 50%, preferably less than 25%, and even more preferably less than 10% mutant or wild type MTRA3 mRNA compared to the level of MTRA3 mRNA in a yeast strain comprising a wild type MTRA3 gene, but otherwise of the same genotype. A yeast strain may be considered to have a total loss of MTRA3 function when the yeast strain comprises less than 5%, preferably less than 1% mutant or wild type MTRA3 mRNA compared to yeast strain comprising a wild type MTRA3 gene, but otherwise of the same genotype. Said mutant MTRA3 is mRNA encoded by a mutated MTRA3 gene carrying a mutation in the mRNA coding region. In one embodiment, wherein said yeast strain is a Dekkera bruxellensis yeast strain, DbMTRA3 mRNA is RNA encoding a polypeptide of SEQ ID NO:26 or a functional homologue thereof, and a wild type DbMTRA3 gene is a gene encoding the polypeptide of SEQ ID NO:26 or a functional homologue thereof. Said functional homologue preferably shares at least 98% sequence identity with SEQ ID NO:26. In one embodiment, a yeast strain with total loss of DbMTRA3 function may contain no detectable mutant or wild type DbMTRA3 mRNA, when determined by conventional quantitative RT-PCR.

In one embodiment, a yeast strain is considered to have a loss of MTRA3 function when the yeast strain comprises less than 50%, preferably less than 25%, and even more preferably less than 10% mutant or wild type MTRA3 protein compared to the level of MTRA3 protein in a yeast strain comprising a wild type MTRA3 gene, but otherwise of the same genotype. A yeast strain may be considered to have a total loss of MTRA3 function when the yeast strain comprises less than 5%, preferably less than 1% mutant or wild type MTRA3 protein compared to a yeast strain comprising a wild type MTRA3 gene, but otherwise of the same genotype. Said mutant MTRA3 protein is a polypeptide encoded by a mutated MTRA3 gene carrying a mutation in the coding region. In one embodiment, wherein said yeast strain is a Dekkera bruxellensis yeast strain, DbMTRA3 protein is a polypeptide of SEQ ID NO:26 or a functional homologue thereof, and a wild type DbMTRA3 gene is a gene encoding the polypeptide of SEQ ID NO:26 or a functional homologue thereof. Said functional homologue preferably shares at least 98% sequence identity with SEQ ID NO:26. In one embodiment, a yeast strain with total loss of DbMTRA3 function may contain no detectable mutant or wild type DbMTRA3 protein as detected by conventional Western blotting.

The yeast strain may for example have genotype VI in embodiments of the invention, where the yeast strain is not capable of utilizing more than 2% maltose.

### Genotype VII—MTRA4

The yeast strain according to the present invention may have the genotype VII, wherein the genotype VII is the presence of one or more mutations in or a deletion of the gene encoding MTRA4.

In embodiments of the invention, wherein the Dekkera yeast strain according to the invention has the genotype VII, said Dekkera yeast strain in general also have characteristic III.

The putative function of MTRA4 is predicted to be a maltose transporter.

In one embodiment, the yeast strain according to the invention lacks the entire DbMTRA4 gene encoding DbMTRA4 of SEQ ID NO:28 or a functional homologue thereof having at least 98% sequence identity herewith.

In another embodiment, the yeast strain lacks a portion of the DbMTRA4 gene hereby encoding only a part of the DbMTRA4, such as at the most 90% of DbMTRA4, such as at the most 80%, such as at the most 70%, such as at the most 60%, such as at the most 50%, such as at the most 40%, such as at the most 30%, such as at the most 30%, such as at the most 20% of DbMTRA4 of SEQ ID NO:28.

In one embodiment, the yeast strain of the invention carries a mutation resulting in a mutant MTRA4 gene encoding a mutant MTRA4. It is preferred that the yeast strain carries a mutation in the MTRA4 gene leading to a loss of MTRA4 function, and in particular to a total loss of MTRA4 function.

The yeast strain carrying a mutation in the MTRA4 gene leading to a loss of MTRA4 function may carry different types of mutations, e.g. any of the mutations described herein in this section.

In one embodiment, the yeast strain of the invention carries a mutation resulting in a mutant MTRA4 gene encoding a mutant MTRA4 protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the yeast strain carries a mutation resulting in a mutant DbMTRA4 gene encoding a mutant DbMTRA4 protein lacking one or more amino acid, such as lacking at least 5 amino acids, such as lacking at least 10, such as lacking at least 15, such as lacking at least 20 amino acids of SEQ ID NO:28.

In one embodiment, the yeast strain of the invention carries a mutation resulting in a mutant DbMTRA4 gene encoding a mutant DbMTRA4 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:28.

In another embodiment, the yeast strain of the invention carries a mutation resulting in a mutant DbMTRA4 gene encoding a mutant DbMTRA4 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:28.

In one embodiment, the yeast strain of the invention carries a mutation resulting in a frame shift mutation of the MTRA4 gene.

In one embodiment, the yeast strain of the invention carries a mutation resulting in formation of a premature stop codon in the MTRA4 gene.

In another embodiment, the mutation is a mutation in a splice site of the MTRA4 gene. Said mutation may lead to aberrant splicing of MTRA4 mRNA.

In one embodiment, the yeast strain carries a mutation in the promoter region of the MTRA4 gene or in an intron of the MTRA4 gene leading to aberrant transcription of MTRA4 mRNA and/or aberrant translation of MTRA4 protein. Such yeast strain may in particular have reduced MTRA4 mRNA levels as described herein below in this section and/or reduced MTRA4 protein levels as described herein below in this section.

Loss of MTRA4 function may be determined by determining by any method known by a person skilled in the art. One way of determining MTRA4 function, can be to determine the expression level of MTRA4 either on the mRNA level or on the protein level.

In one embodiment, a yeast strain is considered to have a loss of MTRA4 function when the yeast strain comprises less than 50%, preferably less than 25%, and even more preferably less than 10% mutant or wild type MTRA4 mRNA compared to the level of MTRA4 mRNA in a yeast strain comprising a wild type MTRA4 gene, but otherwise of the same genotype. A yeast strain may be considered to have a total loss of MTRA4 function when the yeast strain comprises less than 5%, preferably less than 1% mutant or wild type MTRA4 mRNA compared to yeast strain comprising a wild type MTRA4 gene, but otherwise of the same genotype. Said mutant MTRA4 is mRNA encoded by a mutated MTRA4 gene carrying a mutation in the mRNA coding region. In one embodiment, wherein said yeast strain is a Dekkera bruxellensis yeast strain, DbMTRA4 mRNA is RNA encoding a polypeptide of SEQ ID NO:28 or a functional homologue thereof, and a wild type DbMTRA4 gene is a gene encoding the polypeptide of SEQ ID NO:28 or a functional homologue thereof. Said functional homologue preferably shares at least 98% sequence identity with SEQ ID NO:28. In one embodiment, a yeast strain with total loss of DbMTRA4 function may contain no detectable mutant or wild type DbMTRA4 mRNA, when determined by conventional quantitative RT-PCR.

In one embodiment, a yeast strain is considered to have a loss of MTRA4 function when the yeast strain comprises less than 50%, preferably less than 25%, and even more preferably less than 10% mutant or wild type MTRA4 protein compared to the level of MTRA4 protein in a yeast strain comprising a wild type MTRA4 gene, but otherwise of the same genotype. A yeast strain may be considered to have a total loss of MTRA4 function when the yeast strain comprises less than 5%, preferably less than 1% mutant or wild type MTRA4 protein compared to a yeast strain comprising a wild type MTRA4 gene, but otherwise of the same genotype. Said mutant MTRA4 protein is a polypeptide encoded by a mutated MTRA4 gene carrying a mutation in the coding region. In one embodiment, wherein said yeast strain is a Dekkera bruxellensis yeast strain, DbMTRA4 protein is a polypeptide of SEQ ID NO:28 or a functional homologue thereof, and a wild type DbMTRA4 gene is a gene encoding the polypeptide of SEQ ID NO:28 or a functional homologue thereof. Said functional homologue preferably shares at least 98% sequence identity with SEQ ID NO:28. In one embodiment, a yeast strain with total loss of DbMTRA4 function may contain no detectable mutant or wild type DbMTRA4 protein as detected by conventional Western blotting.

The yeast strain may for example have genotype VII in embodiments of the invention, where the yeast strain is not capable of utilizing more than 2% maltose.

### Genotype VIII—MTRA5

The yeast strain according to the present invention may have the genotype VIII, wherein the genotype VIII is the presence of one or more mutations in or a deletion of the gene encoding MTRA5.

In embodiments of the invention, wherein the Dekkera yeast strain according to the invention has the genotype VIII, said Dekkera yeast strain in general also have characteristic III.

The putative function of MTRA5 is predicted to be a high-affinity maltose transporter.

In one embodiment, the yeast strain according to the invention lacks the entire DbMTRA5 gene encoding DbMTRA5 of SEQ ID NO:30 or a functional homologue thereof having at least 98% sequence identity herewith.

In another embodiment, the yeast strain lacks a portion of the DbMTRA5 gene hereby encoding only a part of the DbMTRA5, such as at the most 90% of DbMTRA5, such as at the most 80%, such as at the most 70%, such as at the most 60%, such as at the most 50%, such as at the most 40%, such as at the most 30%, such as at the most 30%, such as at the most 20% of DbMTRA5 of SEQ ID NO:30.

In one embodiment, the yeast strain of the invention carries a mutation resulting in a mutant MTRA5 gene encoding a mutant MTRA5. It is preferred that the yeast strain carries a mutation in the MTRA5 gene leading to a loss of MTRA5 function, and in particular to a total loss of MTRA5 function.

The yeast strain carrying a mutation in the MTRA5 gene leading to a loss of MTRA5 function may carry different types of mutations, e.g. any of the mutations described herein in this section.

In one embodiment, the yeast strain of the invention carries a mutation resulting in a mutant MTRA5 gene encoding a mutant MTRA5 protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the yeast strain carries a mutation resulting in a mutant DbMTRA5 gene encoding a mutant DbMTRA5 protein lacking one or more amino acid, such as lacking at least 5 amino acids, such as lacking at least 10, such as lacking at least 15, such as lacking at least 20 amino acids of SEQ ID NO:30.

In one embodiment, the yeast strain of the invention carries a mutation resulting in a mutant DbMTRA5 gene encoding a mutant DbMTRA5 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:30.

In another embodiment, the yeast strain of the invention carries a mutation resulting in a mutant DbMTRA5 gene encoding a mutant DbMTRA5 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:30.

In one embodiment, the yeast strain of the invention carries a mutation resulting in a frame shift mutation of the MTRA5 gene.

In one embodiment, the yeast strain of the invention carries a mutation resulting in formation of a premature stop codon in the MTRA5 gene.

In another embodiment, the mutation is a mutation in a splice site of the MTRA5 gene. Said mutation may lead to aberrant splicing of MTRA5 mRNA.

In one embodiment, the yeast strain carries a mutation in the promoter region of the MTRA5 gene or in an intron of the MTRA5 gene leading to aberrant transcription of MTRA5 mRNA and/or aberrant translation of MTRA5 protein. Such yeast strain may in particular have reduced MTRA5 mRNA levels as described herein below in this section and/or reduced MTRA5 protein levels as described herein below in this section.

Loss of MTRA5 function may be determined by determining by any method known by a person skilled in the art. One way of determining MTRA5 function, can be to determine the expression level of MTRA5 either on the mRNA level or on the protein level.

In one embodiment, a yeast strain is considered to have a loss of MTRA5 function when the yeast strain comprises less than 50%, preferably less than 25%, and even more preferably less than 10% mutant or wild type MTRA5 mRNA compared to the level of MTRA5 mRNA in a yeast strain comprising a wild type MTRA5 gene, but otherwise of the same genotype. A yeast strain may be considered to have a total loss of MTRA5 function when the yeast strain comprises less than 5%, preferably less than 1% mutant or wild type MTRA5 mRNA compared to yeast strain comprising a wild type MTRA5 gene, but otherwise of the same genotype. Said mutant MTRA5 is mRNA encoded by a mutated MTRA5 gene carrying a mutation in the mRNA coding region.

In one embodiment, wherein said yeast strain is a Dekkera bruxellensis yeast strain, DbMTRA5 mRNA is RNA encoding a polypeptide of SEQ ID NO:30 or a functional homologue thereof, and a wild type DbMTRA5 gene is a gene encoding the polypeptide of SEQ ID NO:30 or a functional homologue thereof. Said functional homologue preferably shares at least 98% sequence identity with SEQ ID NO:30. In one embodiment, a yeast strain with total loss of DbMTRA5 function may contain no detectable mutant or wild type DbMTRA5 mRNA, when determined by conventional quantitative RT-PCR.

In one embodiment, a yeast strain is considered to have a loss of MTRA5 function when the yeast strain comprises less than 50%, preferably less than 25%, and even more preferably less than 10% mutant or wild type MTRA5 protein compared to the level of MTRA5 protein in a yeast strain comprising a wild type MTRA5 gene, but otherwise of the same genotype. A yeast strain may be considered to have a total loss of MTRA5 function when the yeast strain comprises less than 5%, preferably less than 1% mutant or wild type MTRA5 protein compared to a yeast strain comprising a wild type MTRA5 gene, but otherwise of the same genotype. Said mutant MTRA5 protein is a polypeptide encoded by a mutated MTRA5 gene carrying a mutation in the coding region. In one embodiment, said yeast strain is a Dekkera bruxellensis yeast strain, DbMTRA5 protein is a polypeptide of SEQ ID NO:30 or a functional homologue thereof, and a wild type DbMTRA5 gene is a gene encoding the polypeptide of SEQ ID NO:30 or a functional homologue thereof. Said functional homologue preferably shares at least 98% sequence identity with SEQ ID NO:30. In one embodiment, a yeast strain with total loss of DbMTRA5 function may contain no detectable mutant or wild type DbMTRA5 protein as detected by conventional Western blotting.

The yeast strain may for example have genotype VIII in embodiments of the invention, where the yeast strain is not capable of utilizing more than 2% maltose.

### Genotype IX— MTRA6

The yeast strain according to the present invention may have the genotype VII, wherein the genotype VII is the presence of one or more mutations in or a deletion of the gene encoding MTRA6.

In embodiments of the invention, wherein the Dekkera yeast strain according to the invention has the genotype iX, said Dekkera yeast strain in general also have characteristic III.

The putative function of MTRA6 is predicted to be a high-affinity maltose transporter.

In one embodiment, the yeast strain according to the invention lacks the entire DbMTRA6 gene encoding DbMTRA6 of SEQ ID NO:32 or a functional homologue thereof having at least 98% sequence identity herewith.

In another embodiment, the yeast strain lacks a portion of the DbMTRA6 gene hereby encoding only a part of the DbMTRA6, such as at the most 90% of DbMTRA6, such as at the most 80%, such as at the most 70%, such as at the most 60%, such as at the most 50%, such as at the most 40%, such as at the most 30%, such as at the most 30%, such as at the most 20% of DbMTRA6 of SEQ ID NO:32.

In one embodiment, the yeast strain of the invention carries a mutation resulting in a mutant MTRA6 gene encoding a mutant MTRA6. It is preferred that the yeast strain carries a mutation in the MTRA6 gene leading to a loss of MTRA6 function, and in particular to a total loss of MTRA6 function.

The yeast strain carrying a mutation in the MTRA6 gene leading to a loss of MTRA6 function may carry different types of mutations, e.g. any of the mutations described herein in this section.

In one embodiment, the yeast strain of the invention carries a mutation resulting in a mutant MTRA6 gene encoding a mutant MTRA6 protein comprising one or more amino acid substitutions, such as 5 or more, such as 10 or more, such as 15 or more, such as 20 or more amino acids substitutions. Said amino acid substitutions may be any amino acid substitutions, wherein the amino acid is replaced with another amino acid.

In one embodiment, the yeast strain carries a mutation resulting in a mutant DbMTRA6 gene encoding a mutant DbMTRA6 protein lacking one or more amino acid, such as lacking at least 5 amino acids, such as lacking at least 10, such as lacking at least 15, such as lacking at least 20 amino acids of SEQ ID NO:32.

In one embodiment, the yeast strain of the invention carries a mutation resulting in a mutant DbMTRA6 gene encoding a mutant DbMTRA6 protein lacking at least the 10 most N-terminal amino acids, for example at least the 20 most N-terminal amino acids, such as at least the 30 most N-terminal amino acids, for example at least the 60 most N-terminal amino acids, such as at least the 100 most N-terminal amino acids of SEQ ID NO:32.

In another embodiment, the yeast strain of the invention carries a mutation resulting in a mutant DbMTRA6 gene encoding a mutant DbMTRA6 protein lacking at least the 10 most C-terminal amino acids, for example at least the 20 most C-terminal amino acids, such as at least the 30 most C-terminal amino acids, for example at least the 60 most C-terminal amino acids, such as at least the 100 most C-terminal amino acids of SEQ ID NO:32.

In one embodiment, the yeast strain of the invention carries a mutation resulting in a frame shift mutation of the MTRA6 gene.

In one embodiment, the yeast strain of the invention carries a mutation resulting in formation of a premature stop codon in the MTRA6 gene.

In another embodiment, the mutation is a mutation in a splice site of the MTRA6 gene. Said mutation may lead to aberrant splicing of MTRA6 mRNA.

In one embodiment, the yeast strain carries a mutation in the promoter region of the MTRA6 gene or in an intron of the MTRA6 gene leading to aberrant transcription of MTRA6 mRNA and/or aberrant translation of MTRA6 protein. Such yeast strain may in particular have reduced MTRA6 mRNA levels as described herein below in this section and/or reduced MTRA6 protein levels as described herein below in this section.

Loss of MTRA6 function may be determined by determining by any method known by a person skilled in the art. One way of determining MTRA6 function, can be to determine the expression level of MTRA6 either on the mRNA level or on the protein level.

In one embodiment, a yeast strain is considered to have a loss of MTRA6 function when the yeast strain comprises less than 50%, preferably less than 25%, and even more preferably less than 10% mutant or wild type MTRA6 mRNA compared to the level of MTRA6 mRNA in a yeast strain comprising a wild type MTRA6 gene, but otherwise of the same genotype. A yeast strain may be considered to have a total loss of MTRA6 function when the yeast strain comprises less than 5%, preferably less than 1% mutant or wild type MTRA6 mRNA compared to yeast strain comprising a wild type MTRA6 gene, but otherwise of the same genotype. Said mutant MTRA6 is mRNA encoded by a mutated MTRA6 gene carrying a mutation in the mRNA coding region.

In one embodiment, wherein said yeast strain is a Dekkera bruxellensis yeast strain, DbMTRA6 mRNA is RNA encoding a polypeptide of SEQ ID NO:32 or a functional homologue thereof, and a wild type DbMTRA6 gene is a gene encoding the polypeptide of SEQ ID NO:32 or a functional homologue thereof. Said functional homologue preferably shares at least 98% sequence identity with SEQ ID NO:32. In one embodiment, a yeast strain with total loss of DbMTRA6 function may contain no detectable mutant or wild type DbMTRA6 mRNA, when determined by conventional quantitative RT-PCR.

In one embodiment, a yeast strain is considered to have a loss of MTRA6 function when the yeast strain comprises less than 50%, preferably less than 25%, and even more preferably less than 10% mutant or wild type MTRA6 protein compared to the level of MTRA6 protein in a yeast strain comprising a wild type MTRA6 gene, but otherwise of the same genotype. A yeast strain may be considered to have a total loss of MTRA6 function when the yeast strain comprises less than 5%, preferably less than 1% mutant or wild type MTRA6 protein compared to a yeast strain comprising a wild type MTRA6 gene, but otherwise of the same genotype. Said mutant MTRA6 protein is a polypeptide encoded by a mutated MTRA6 gene carrying a mutation in the coding region. In one embodiment, said yeast strain is a Dekkera bruxellensis yeast strain, DbMTRA6 protein is a polypeptide of SEQ ID NO:32 or a functional homologue thereof, and a wild type DbMTRA6 gene is a gene encoding the polypeptide of SEQ ID NO:32 or a functional homologue thereof. Said functional homologue preferably shares at least 98% sequence identity with SEQ ID NO:32. In one embodiment, a yeast strain with total loss of DbMTRA6 function may contain no detectable mutant or wild type DbMTRA6 protein as detected by conventional Western blotting.

The yeast strain may for example have genotype IX in embodiments of the invention, where the yeast strain is not capable of utilizing more than 2% maltose.

Malt and/or Cereal Based Beverage and Methods of Production Thereof

The invention provides a Dekkera yeast strain described herein above, as well as methods of preparing malt and/or cereal based beverages, using said yeast strain.

It is an aspect of the invention to provide methods of producing a malt and/or cereal based beverage, said method comprising the steps of
i) providing an aqueous extract of malt and/or cereal kernels
ii) providing a Dekkera yeast strain, wherein said yeast strain is not capable of converting more than 25% of p-coumaric acid into 4-ethylphenol when incubated in an aqueous solution comprising p-coumaric acid
iii) fermenting said aqueous extract with said yeast
   thereby obtaining said malt and/or cereal based beverage.

It is a further aspect of the invention to provide a malt and/or cereal based beverage comprising less than 3% ethanol, said method comprising the steps of
i) providing an aqueous extract of malt and/or cereal kernels
ii) providing a Dekkera yeast strain, wherein said yeast strain is not capable of converting more than 25% of p-coumaric acid into 4-ethylphenol when incubated in an aqueous solution comprising p-coumaric acid, wherein said yeast strain is furthermore not capable of utilizing more than 2% maltose
iii) fermenting said aqueous extract with said yeast
   thereby obtaining said malt and/or cereal based beverage.

The aqueous extract, may be any aqueous extract of malt and/or cereal kernels. Thus, non-limiting examples hereof are wort and fermented malt and/or cereal based beverages, such as beer. The aqueous extract may for example be prepared by preparing an extract of malt by mashing and optionally sparging as described herein in this section below.

Malt is kernels that have been malted, such as barely kernels. By the term “malting” is to be understood germination of steeped kernels in a process taking place under controlled environmental conditions, followed by a drying step. Said drying step may preferably be kiln drying of the germinated kernels at elevated temperatures.

This aforementioned sequence of malting events is important for the synthesis of numerous enzymes that cause kernel modification, processes that principally depolymerize strain walls of the dead endosperm to mobilize the kernel nutrients and activate other depolymerases. In the subsequent drying process, flavour and colour are generated due to chemical browning reactions.

Steeping may be performed by any conventional method known to the skilled person. One non-limiting example involves steeping at a temperature in the range of 10 to 25° C. with alternating dry and wet conditions. Germination may be performed by any conventional method known to the skilled person. One non-limiting example involves germination at a temperature in the range of 10 to 25° C., optionally with changing temperature in the range of 1 to 4 h.

The kiln drying may be performed at conventional temperatures, such as at least 75° C., for example in the range of 80 to 90° C., such as in the range of 80 to 85° C. Thus, the malt may, for example be produced by any of the methods described by Briggs et al. (1981) and by Hough et al. (1982). However, any other suitable method for producing malt may also be used with the present invention, such as methods for production of specialty malts, including, but not limited to, methods of roasting the malt.

Malt may be further processed, for example by milling. Preferably milling is performed in a dry state, i.e. the malt is milled while dry.

The malt, e.g. the milled malt may be mashed to prepare an aqueous extract of said malt. The starting liquid for preparing the beverage may be an aqueous extract of malt, e.g. an aqueous extract of malt prepared by mashing.

Thus, the method for preparing a malt and/or cereal based beverage according to the invention may comprise a step of producing an aqueous extract, such as wort, by mashing malt and optionally additional adjuncts. Said mashing step may also optionally comprise sparging, and accordingly said mashing step may be a mashing step including a sparging step or a mashing step excluding a sparging step.

In general, the production of the aqueous extract is initiated by the milling of malt and/or kernels. If additional adjuncts are added, these may also be milled depending on their nature. If the adjunct is a cereal, it may for example be milled, whereas syrups, sugars and the like will generally not be milled. Milling will facilitate water access to kernel particles in the mashing phase. During mashing enzymatic depolymerization of substrates initiated during malting may be continued.

In general, the aqueous extract is prepared by combining and incubating milled malt and water, i.e. in a mashing process. During mashing, the malt/liquid composition may be supplemented with additional carbohydrate-rich adjunct compositions, for example milled barley, maize, or rice adjuncts. Unmalted cereal adjuncts usually contain little or no active enzymes, making it important to supplement with malt or exogenous enzymes to provide enzymes necessary for polysaccharide depolymerization etc.

During mashing, milled malt and/or milled kernels—and optionally additional adjuncts are incubated with a liquid fraction, such as water. The incubation temperature is in general either kept constant (isothermal mashing), or gradually increased, for example increased in a sequential manner. In either case, soluble substances in the malt/kernel/adjuncts are liberated into said liquid fraction. A subsequent filtration confers separation of the aqueous extract and residual solid particles, the latter also denoted “spent kernel”. The aqueous extract thus obtained may also be denoted “first wort”. Additional liquid, such as water may be added to the spent kernels during a process also denoted sparging. After sparging and filtration, a “second wort” may be obtained. Further worts may be prepared by repeating the procedure. Non-limiting examples of suitable procedures for preparation of wort is described by Briggs et al. (supra) and Hough et al. (supra).

As mentioned above, the aqueous extract may also be prepared by mashing unmalted kernels. Unmalted kernels lack or contain only a limited amount of enzymes beneficial for wort production, such as enzymes capable of degrading strain walls or enzymes capable of depolymerising starch into sugars. Thus, in embodiments of the invention where unmalted kernels, such as barley kernels, are used for mashing, it is preferred that one or more suitable, external brewing enzymes are added to the mash. Suitable enzymes may be lipases, starch degrading enzymes (e.g. amylases), glucanases [preferably (1-4)- and/or (1-3,1-4)-p-glucanase], and/or xylanases (such as arabinoxylanase), and/or proteases, or enzyme mixtures comprising one or more of the aforementioned enzymes, e.g. Cereflo, Ultraflo, or Ondea Pro (Novozymes).

The aqueous extract may also be prepared by using a mixture of malted and unmalted kernels, in which case one or more suitable enzymes may be added during preparation. More specifically, kernels can be used together with malt in any combination for mashing—with or without external brewing enzymes—such as, but not limited to, the proportions of kernel:malt=approximately 100:0, or approximately 75:25, or approximately 50:0, or approximately 25:75.

In other embodiments of the invention, it is preferred that no external enzymes, in particular that no external protease, and/or no external celluluase and/or no external α-amylase and/or no external p-amylase and/or no external maltogenic α-amylase is added before or during mashing.

The aqueous extract obtained after mashing may also be referred to as “sweet wort”. In conventional methods, the sweet wort is boiled with or without hops where after it may be referred to as boiled wort.

The term “approximately” as used herein means±10%, preferably ±5%, yet more preferably ±2%.

The aqueous extract may be heated or boiled before it is subjected to fermentation with the yeast of the invention. In one aspect of the invention, second and further worts may be combined, and thereafter subjected to heating or boiling. The aqueous extract may be heated or boiled for any suitable amount of time, e.g. in the range of 60 min to 120 min.

The outcome of the fermented malt and/or cereal based beverages is highly dependent on the amount and type of aromatic precursors, such as different phenolic compounds, such as p-coumaric acid and ferulic acid, as well as the characteristics of the yeast strain used during fermentation. The outcome of the fermented malt and/or cereal based beverages is also highly dependent of fermentable sugars present in the aqueous extract of malt and/or cereal kernels.

In one aspect of the present invention, the aqueous extract used in the method of the present invention may comprise p-coumaric acid, such as in the range of 0.1 to 100 mg/L p-coumaric acid, such as 0.2 mg/L to 50 mg/L, such as 0.5 to 20 mg/L, such as 1 to 5 mg/L p-coumaric acid.

In another aspect of the present invention, the aqueous extract comprises ferulic acid, such as in the range of 0.1 to 100 mg/L ferulic acid, such as 0.2 mg/L to 50 mg/L, such as 0.5 to 20 mg/L, such as 1 to 5 mg/L ferulic acid.

In one aspect of the present invention, the aqueous extract used in the method of the present invention may have a sugar content in the range of 7 to 11°Plato, such as in the range of 8 to 10°Plato, such as approx. 9° Plato.

The aqueous extract used in the present invention may contain more than 40 g/kg maltose. In one embodiment, the aqueous extract comprises 40 to 100 g/kg maltose.

The aqueous extract used in the present invention may also contain 8 to 20 g/kg maltotriose, such as 10 to 18 g/kg maltotriose.

The aqueous extract used in the present invention may also contain 1 to 5 g/kg maltotetraose, such as 2 to 4 g/kg maltotetraose.

The aqueous extract used in the method of the present invention may comprise at the most 25 g/kg glucose, such as at the most 20 g/kg, such as at the most 15 g/kg, such as at the most 10 g/kg, and for example such as at the most 5 g/L glucose.

In one embodiment, the aqueous solution comprises in the range of 8 to 50 g/kg glucose, preferably in the range of 1 to 30 g/kg glucose, such as in the range of 1 to 10 g/kg glucose.

It is preferred that, an aqueous extract used to prepare a low-alcohol and/or alcohol-free beverage contains at the most 10 g/L glucose.

Thus, the aqueous extract is prepared as described above. The malt and/or cereal based beverage may be prepared by fermentation of said aqueous extract with said yeast strain according to the invention.

The malt and/or cereal based beverage may in one preferred embodiment be a beer. The fermented malt and/or cereal based beverage may in some embodiments be a low-alcohol malt and/or cereal based beverage or an alcohol-free malt and/or cereal based beverage, such as low-alcohol beer or alcohol-free beer.

In one embodiment the beverage is a beer, for example the beer may be a Lager, Saison, Belgian ale, India Pale ale, Weissbier, Dunkel, Porter, Lambic or Kriek type of beer, with a low alcohol percentage.

In general terms, alcoholic beverages—such as beer—may be manufactured from malted and/or unmalted kernels. Malt, in addition to hops and yeast, contributes to flavor and color of the beverage, such as beer. Furthermore, malt functions as a source of fermentable sugar and enzymes. Non-limited descriptions of examples of suitable methods for malting and brewing can be found, for example, in publications by Briggs et al. (1981) and Hough et al. (1982). Numerous, regularly updated methods for analyses of kernel, malt and beer products are available, for example, but not limited to, American Association of Cereal Chemists (1995), American Society of Brewing Chemists (1992), European Brewery Convention (1998), and Institute of Brewing (1997). It is recognized that many specific procedures are employed for a given brewery, with the most significant variations relating to local consumer preferences. Any such method of producing beer may be used with the present invention.

The first step of producing beer from wort preferably involves heating said wort as described herein above, followed by a subsequent phase of wort cooling and optionally whirlpool rest.

The methods of the invention comprises a step of fermenting an aqueous extract of malt and/or cereal kernels with the yeast strain according to the invention. Said fermentation may be a fermentation of an unfermented aqueous extract or a fermented aqueous extract. Thus, in some embodiments said fermentation may be performed essentially immediately after completion of mashing or after heating of wort. Fermentation of an unfermented aqueous extract may also be referred to as “primary fermentation” herein. However, in other embodiments the aqueous extract is a fermented aqueous extract, which has been subjected to a step of fermentation with another microorganism first. Such fermentation may also be referred to as “secondary fermentation” herein. It is also comprised within the invention that said step of fermenting the aqueous extract is performed in the presence of a plurality of different microorganisms, wherein at least one is a Dekkera yeast strain according to the invention.

Fermentation, e.g. primary and/or secondary fermentation may be performed in fermentation tanks containing yeast according to the invention, i.e. yeast having one or more of the characteristics described above. The wort will be fermented for any suitable time period, in general in the range of 1 to 100 days, such as in the range of 1 to 21 days, such as 2 to 10 days, such as 3 to 7 days. The fermentation is performed at any useful temperature e.g. at a temperature in the range of 5 to 30° C., such as 10 to 28° C., such as 15 to 25° C.

Thus, the fermentation in step iii) described above is carried out by fermenting an aqueous extract with a Dekkera yeast strain as described above.

In one embodiment the aqueous extract is wort, thus the fermentation can be considered to be primary fermentation.

In another embodiment, the aqueous extract is a fermented malt and/or cereal based beverage, such as beer, thus the fermentation can be considered to be secondary fermentation.

During the several-day-long fermentation process, flavor substances are developed. If the yeast strain is not capable of converting specific compounds, these will still be present after the fermentation step iii).

In addition to the flavor substance development during the fermentation process, the fermentable sugar(s) which can be utilized by the yeast strain is converted to ethanol and CO₂ concomitantly with the development of flavor substances. If the yeast strain is not capable of fermenting specific fermentable sugars, these will still be present after the fermentation step iii) and little or no ethanol will be produced.

In one aspect of the present invention, the malt and/or cereal based beverage produced by the method of the present invention may comprises low levels of 4-ethylphenol. In one embodiment said malt and/or cereal based beverage comprises less than 0.5 mg/L of 4-ethylphenol, such as less than 0.3 mg/L, such as less than 0.1 mg/L 4-ethylphenol.

In another aspect of the present invention, the malt and/or cereal based beverage produced by the method of the present invention may comprise low levels of 4-ethylguaiacol. In one embodiment, said malt and/or cereal based beverage comprises less than 1 mg/L of 4-ethylguaiacol, such as less than 0.8 mg/L, such as less than 0.6 mg/L, such as less than 0.5 mg/L of 4-ethylguaiacol

In another embodiment, the malt and/or cereal based beverage produced according to the method of the invention comprises less than 3% ethanol, such as less than 2% ethanol, such as less than 1.5% ethanol, such as less than 1.0% ethanol, such as less than 0.5% ethanol, such as less than 0.3% ethanol, such as less than 0.1% ethanol.

Subsequently, malt and/or cereal based beverage may be further processed. In one embodiment of the present invention, the malt and/or cereal based beverage is diluted with a liquid, such as water.

Optionally, water can be used to dilute the malt and/or cereal based beverage and thereby adjust e.g. the ethanol content. In one embodiment of the present invention the proportions of water:malt and/or cereal based beverage may be in the range of 0.1 to 5 parts water to 1 part malt and/or cereal based beverage.

In one embodiment the malt and/or cereal based beverage is diluted with water, so the final ethanol concentration of the malt and/or cereal based beverage is below 1.9% ethanol, such as below 1.5% ethanol, such as below 1.0% ethanol, such as below 0.5% ethanol, such as below 0.3% ethanol, such below 0.1% ethanol.

The further process may for example also include chilling and/or filtering of the malt and/or cereal based beverage. Also additives may be added. Furthermore, CO₂ may be added. Finally, the malt and/or cereal based beverage, such as a beer, may be pasteurized and/or filtered, before it is packaged (e.g. bottled or canned).

The malt and/or cereal based beverage produced by fermentation with the yeasts according to the invention in general has a superior pleasant taste and low ethanol content. Taste may be analyzed, for example, by a specialist beer taste panel. Preferably, said panel is trained in tasting and describing beer flavors, with special focus on aldehydes, papery taste, old taste, esters, higher alcohols, fatty acids and sulphury components.

In general, the taste panel will consist of in the range of 3 to 30 members, for example in the range of 5 to 15 members, preferably in the range of 8 to 12 members. The taste panel may evaluate the presence of various flavours, such as papery, oxidized, aged, and bready off-flavours as well as flavours of esters, higher alcohols, sulfur components and body of beer.

The present invention also provides malt and/or cereal based beverages, prepared by the methods described above.

In another aspect of the present invention, the malt and/or cereal based beverage, produced by fermenting the aqueous extract with said yeast strain according to the present invention has a pleasant taste with reduced levels of phenolic off-flavors.

In one embodiment, the malt and/or cereal based beverage produced according to the method of the invention comprises less than 3% ethanol, such as less than 2% ethanol, such as less than 1.5% ethanol, such as less than 1.0% ethanol, such as less than 0.5% ethanol, such as less than 0.3% ethanol, such as less than 0.1% ethanol.

In another aspect of the present invention, the malt and/or cereal based beverage, produced by fermenting the aqueous extract with said yeast strain according to the present invention has a pleasant taste.

In one embodiment of the present invention, the malt and/or cereal based beverage has a p-citronellol concentration of less than 25 μg/L of, such as less than 20 μg/L.

In another embodiment, the malt and/or cereal based beverages has a geraniol concentration of at least 18 μg/L of, such as at least 20 μg/L.

### Sequence Listing

| | |
|---|---|
| SEQ ID NO: 1 | Nucleotide sequence of DaPAD1 in Dekkera anomalus |
| SEQ ID NO: 2 | Amino acid sequence of DaPAD1 in Dekkera anomalus |
| SEQ ID NO: 3 | Nucleotide sequence of DaSOD in Dekkera anomalus |
| SEQ ID NO: 4 | Amino acid sequence of DaSOD in Dekkera anomalus |
| SEQ ID NO: 5 | Nucleotide sequence of DbPAD2 in Dekkera bruxellensis |
| SEQ ID NO: 6 | Amino acid sequence of DbPAD2 in Dekkera bruxellensis |
| SEQ ID NO: 7 | Nucleotide sequence of DbSOD in Dekkera bruxellensis |
| SEQ ID NO: 8 | Amino acid sequence of DbSOD in Dekkera bruxellensis |
| SEQ ID NO: 9 | Nucleotide sequence of DaMTRA1 in Dekkera anomalus |
| SEQ ID NO: 10 | Amino acid sequence of DaMTRA1 in Dekkera anomalus |
| SEQ ID NO: 11 | Nucleotide sequence of DalSOM in Dekkera anomalus |
| SEQ ID NO: 12 | Amino acid sequence of DalSOM in Dekkera anomalus |
| SEQ ID NO: 13 | Nucleotide sequence of DaMTRA2 in Dekkera anomalus |
| SEQ ID NO: 14 | Amino acid sequence of DaMTRA2 in Dekkera anomalus |
| SEQ ID NO: 15 | Nucleotide sequence of DbMTRA1 in Dekkera bruxellensis |
| SEQ ID NO: 16 | Amino acid sequence of DbMTRA1 in Dekkera bruxellensis |
| SEQ ID NO: 17 | Nucleotide sequence of DblSOM(2) in Dekkera bruxellensis |
| SEQ ID NO: 18 | Amino acid sequence of DblSOM(2) in Dekkera bruxellensis |
| SEQ ID NO: 19 | Nucleotide sequence of DbMTRA2 in Dekkera bruxellensis |
| SEQ ID NO: 20 | Amino acid sequence of DbMTRA2 in Dekkera bruxellensis |
| SEQ ID NO: 21 | Nucleotide sequence of DblSOM(1) in Dekkera bruxellensis |
| SEQ ID NO: 22 | Amino acid sequence of DblSOM(1) in Dekkera bruxellensis |
| SEQ ID NO: 23 | Nucleotide sequence of DbPAD1 in Dekkera bruxellensis |
| SEQ ID NO: 24 | Amino acid sequence of DbPAD1 in Dekkera bruxellensis |
| SEQ ID NO: 25 | Nucleotide sequence of DbMTRA3 in Dekkera bruxellensis |
| SEQ ID NO: 26 | Amino acid sequence of DbMTRA3 in Dekkera bruxellensis |
| SEQ ID NO: 27 | Nucleotide sequence of DbMTRA4 in Dekkera bruxellensis |
| SEQ ID NO: 28 | Amino acid sequence of DbMTRA4 in Dekkera bruxellensis |
| SEQ ID NO: 29 | Nucleotide sequence of DbMTRA5 in Dekkera bruxellensis |
| SEQ ID NO: 30 | Amino acid sequence of DbMTRA5 in Dekkera bruxellensis |
| SEQ ID NO: 31 | Nucleotide sequence of DbMTRA6 in Dekkera bruxellensis |
| SEQ ID NO: 32 | Amino acid sequence of DbMTRA7 in Dekkera bruxellensis |

### REFERENCES

Briggs, D. E. et al. Malting and Brewing science. 1981.
Daenen L et al. 2008: Screening and evaluation of the glucoside hydrolase activity in Saccharomyces and Brettanomyces brewing yeasts. J Appl Microbiol 2008, 104:478-488.
Harris et al. “Survey of enzyme activity responsible for phenolic off-flavour production by Dekkera and Brettanomyces yeasts. Vol. 81, no. 6. A January 2009.
Hough, J. S. et al. Malting and Brewing science: Hopped Wort and Beer, Volume 2. 1982.
Li et al. (2015 Apr. 6) Nucleic Acids Research 43 (W1):W580-4 PMID: 25845596; McWilliam et al., (2013 May 13) Nucleic Acids Research 41 (Web Server issue):W597-600 PMID: 23671338
Mukai et al. PAD1 and FDC1 are essential for the decarboxylation of phenylacrylic acids in Saccharomyces cerevisiae. Journal of Bioscience and Bioengineering Vol 109, no. 6, 1 Jun. 2010.
Pinu F R, Villas-Boas S G: Rapid quantification of major volatile metabolites in fermented food and beverages using gas chromatography-mass spectrometry. Metabolites 2017, 7.
Sievers et al. (2011 Oct. 11) Molecular Systems Biology 7:539, PMID: 21988835

### Items

The invention may furthermore be defined by any one of the following items:
1. A method of producing a malt and/or cereal based beverage with low levels of 4-ethylphenol, said method comprising the steps of
   i) providing an aqueous extract of malt and/or cereal grains
   ii) providing a Dekkera yeast strain, wherein said yeast carries a mutation in or a deletion of one of the following genes:
      a. PAD
      b. SOD
   iii) fermenting said aqueous extract with said yeast
   thereby obtaining said malt and/or cereal based beverage.
2. A method of producing a malt and/or cereal based beverage, said method comprising the steps of
   i) providing an aqueous extract of malt and/or cereal kernels
   ii) providing a Dekkera yeast strain, wherein said yeast strain is not capable of converting more than 25% of p-coumaric acid into 4-ethylphenol when incubated in an aqueous solution comprising p-coumaric acid
   iii) fermenting said aqueous extract with said yeast
   thereby obtaining said malt and/or cereal based beverage.
3. The method according to item 2, wherein said yeast strain is not capable of converting more than 25%, such as not more than 20%, such as not more than 15%, such as not more than 10%, such as not more than 5%, such as not more than 1% of the p-coumaric acid present in the aqueous solution into 4-vinylphenol.
4. A method of producing a malt and/or cereal based beverage, said method comprising the steps of
   i) providing an aqueous extract of malt and/or cereal kernels
   ii) providing a Dekkera yeast strain, wherein said yeast strain is not capable of converting more than 25% of ferulic acid into 4-ethylguaiacol when incubated in an aqueous solution comprising ferulic acid
   iii) fermenting said aqueous extract with said yeast
   thereby obtaining said malt and/or cereal based beverage.
5. The method according to item 4, wherein said yeast strain is not capable of converting more than 25%, such as not more than 20%, such as not more than 15%, such as not more than 10%, such as not more than 5%, such as not more than 1% of the ferulic acid present in the aqueous solution into 4-vinylguaiacol.
6. The method according to any one of the preceding items, wherein said yeast strain has the genotype I and/or the genotype II:
   I: comprising a mutation in or a deletion of the gene encoding PAD
   II: comprising a mutation in or a deletion of the gene encoding SOD.
7. The method according to any one of the preceding items, wherein the yeast strain is a Dekkera anomalus yeast strain, said yeast strain has the genotype I:
   I: comprising a mutation in or a deletion of the gene encoding DaPAD1 of SEQ ID NO:2 or a functional homologue thereof having at least 80%, such as at least 90%, for example at least 95% sequence identity herewith.
8. The method according to any one of the preceding items, wherein the yeast strain is a Dekkera anomalus yeast strain, said yeast strain has the genotype I:
   I: comprising a mutation in or a deletion of the gene encoding DaPAD1 of SEQ ID NO:2.
9. The method according to any one of the preceding items, wherein the yeast strain is a Dekkera bruxellensis yeast strain, said yeast strain has the genotype I:
   I: comprising a mutation in or a deletion of the gene encoding DbPAD2 of SEQ ID NO:6 or a functional homologue thereof having at least 80%, such as at least 90%, for example at least 95% sequence identity herewith.
10. The method according to any one of the preceding items, wherein the yeast strain is a Dekkera bruxellensis yeast strain, said yeast strain has the genotype I:
   I: comprising a mutation in or a deletion of the gene encoding DbPAD2 of SEQ ID NO:6.
11. The method according to any one of the preceding items, wherein the yeast strain is a Dekkera anomalus yeast strain, said yeast strain has the genotype II:
   II: comprising a mutation in or a deletion of the gene encoding DaSOD of SEQ ID NO:4 or a functional homologue thereof having at least 80%, such as at least 90%, for example at least 95% sequence identity herewith.
12. The method according to any one of the preceding items, wherein the yeast strain is a Dekkera bruxellensis yeast strain, said yeast strain has the genotype II:
   II: comprising a mutation in or a deletion of the gene encoding DbSOD of SEQ ID NO:8 or a functional homologue thereof having at least 80%, such as at least 90%, for example at least 95% sequence identity herewith.
13. The method according to any one of the preceding items, wherein the aqueous extract comprises p-coumaric acid, such as in the range of 0.1 to 100 mg/L p-coumaric acid, such as in the range of 0.2 mg/L to 50 mg/L, such as in the range of 0.5 to 20 mg/L, such as in the range of 1 to 5 mg/L p-coumaric acid.
14. The method according to any one of the preceding items, wherein said yeast strain is not capable of converting more than 20%, such as not more than 15%, such as not more than 10%, such as not more than 5%, such as not more than 1%, of the p-coumaric acid present in the aqueous extract into 4-ethylphenol.
15. The method according to any one of the preceding items, wherein said yeast strain when incubated in an aqueous solution comprising a predetermined level of p-coumaric acid is not capable of reducing the level of p-coumaric acid by more than 25%, for example not by more than 20%, such as not by more than 15%, such as not by more than 10%, such as not by more than 5%, such as not by more than 1%.
16. The method according to any one of the preceding items, wherein said yeast strain when incubated in an aqueous solution containing a predetermined level of p-coumaric acid and a predetermined level of 4-ethylphenol is not capable of increasing the molar 4-ethylphenol level by more than 25%, such as not more than 20%, such as not more than 15%, for example not more than 10%, such as not more than 5%, for example not more than 1% of the predetermined molar level of p-coumaric acid
17. The method according to any one of the preceding items, wherein said malt and/or cereal based beverage comprises low levels of 4-ethylphenol.
18. The method according to any one of the preceding items, wherein said malt and/or cereal based beverage comprises less than 0.5 mg/L of 4-ethylphenol, such as less than 0.3 mg/L, such as less than 0.1 mg/L 4-ethylphenol.
19. The method according to any one of the preceding items, wherein the aqueous extract comprises ferulic acid, such as in the range of 0.1 to 100 mg/L ferulic acid, such as 0.2 mg/L to 50 mg/L, such as 0.5 to 20 mg/L, such as 1 to 5 mg/L ferulic acid.
20. The method according to any one of the preceding items, wherein said yeast strain is not capable of converting more than 25% of the ferulic acid present in the aqueous extract into 4-ethylguaiacol.
21. The method according to any one of the preceding items, wherein said yeast strain is not capable of converting more than 20%, such as not more than 15%, such as not more than 10%, such as not more than 5%, such as not more than 1% of the ferulic acid present in the aqueous extract into 4-ethylguaiacol.
22. The method according to any one of the preceding items, wherein said yeast strain when incubated in an aqueous solution comprising a predetermined level of ferulic acid is not capable of reducing the level of ferulic acid by more than 25%, for example not by more than 20%, such as not by more than 15%, such as not by more than 10%, such as not by more than 5%, such as not by more than 1%.
23. The method according to any one of the preceding items, wherein said yeast strain when incubated in an aqueous solution containing a predetermined level of ferulic acid and a predetermined level of 4-ethylguaiacol is not capable of increasing the molar 4-ethylguaiacol level by more than 25%, such as not more than most 20%, such as not more than 15%, for example not more than 10%, such as not more than 5%, for example not more than 1% of the predetermined molar level of p-coumaric acid.
24. The method according to any one of the preceding items, wherein said malt and/or cereal based beverage comprises low levels of 4-ethylguaiacol.
25. The method according to any one of the preceding items, wherein said malt and/or cereal based beverage comprises less than 1 mg/L of 4-ethylguaiacol, such as less than 0.8 mg/L, such as less than 0.6 mg/L, such as less than 0.5 mg/L of 4-ethylguaiacol.
26. The method according to any one of the preceding items, wherein the yeast is Dekkera anomalus.
27. The method according to any one of the preceding items, wherein the yeast strain is of the species Dekkera anomalus, and said yeast strain carries a mutation in the DaPAD1 gene resulting in a mutant DaPAD1 gene encoding a mutant DaPAD1 protein lacking one or more of the amino acids of SEQ ID NO:2.
28. The method according to any one of the preceding items, wherein the yeast strain is of the species Dekkera anomalus, and said yeast strain carries one or more of the following mutations
   i. a mutation introducing a premature stop codon in the DaPAD1 gene
   ii. a mutation in a splice site of the DaPAD1 gene
   iii. a mutation in the DaPAD1 gene resulting in a frameshift mutation
   iv. a mutation resulting in a deletion of a part of the DaPAD1 gene,
   wherein the wild type DaPAD1 gene encodes a polypeptide of SEQ ID NO:2.
29. The method according to any one of the preceding items, wherein the yeast strain is of the species Dekkera anomalus, and said yeast strain comprises a mutant DaPAD1 gene encoding a mutant DaPAD1 protein lacking at least 50 amino acids, such as at least 70 amino acids, such as at least 100 amino acids, such as at least 150 amino acids of SEQ ID NO: 2.
30. The method according to any one of the preceding items, wherein the yeast strain is of the species Dekkera anomalus, and said yeast strain comprises a mutant DaPAD1 gene encoding a mutant DaPAD1 protein lacking at least the 50 most C-terminal amino acids, such as at least the 100 most C-terminal amino acids, such as at least 150 most C-terminal amino acids of SEQ ID NO: 2.
31. The method according to any one of the preceding items, wherein the yeast strain is of the species Dekkera bruxellensis, and said yeast strain carries one or more of the following mutations
   i. a mutation introducing a premature stop codon in the DbPAD2 gene
   ii. a mutation in a splice site of the DbPAD2 gene
   iii. a mutation in the DbPAD2 gene resulting in a frameshift mutation
   iv. a mutation resulting in a deletion of a part of the DbPAD2 gene,
   wherein the wild type DbPAD2 gene encodes a polypeptide of SEQ ID NO:6.
32. The method according to any one of the preceding items, wherein the yeast strain is of the species Dekkera bruxellensis, and said yeast strain comprises a mutant DbPAD2 gene encoding a mutant DbPAD2 protein lacking at least 50 amino acids, such as at least 70 amino acids, such as at least 100 amino acids, such as at least 150 amino acids of SEQ ID NO:6.
33. The method according to any one of the preceding items, wherein the yeast strain is of the species Dekkera bruxellensis, and said yeast strain comprises a mutant DbPAD2 gene encoding a mutant DbPAD2 protein lacking at least the 50 most C-terminal amino acids, such as at least the 100 most C-terminal amino acids, such as at least 150 most C-terminal amino acids of SEQ ID NO:6.
34. The method according to any one of the preceding items, wherein the yeast strain carries a mutant PAD gene comprising a mutant PAD promoter.
35. The method according to any one of the preceding items, wherein the yeast strain carries a mutation in the PAD gene leading to loss of PAD function.
36. The method according to any one of the preceding items, wherein the yeast strain carries a mutation in the SOD gene resulting in a mutant SOD gene encoding a mutant SOD protein lacking one or more of the amino acids.
37. The method according to any one of the preceding items, wherein the yeast strain is of the species Dekkera anomalus, and said yeast strain carries a mutation in the DaSOD gene resulting in a mutant DaSOD gene encoding a mutant DaSOD protein lacking one or more of the amino acids of SEQ ID NO:4.
38. The method according to any one of the preceding items, wherein the yeast strain is of the species Dekkera anomalus, and said yeast strain carries one or more of the following mutations
   i. a mutation introducing a premature stop codon in the DaSOD gene
   ii. a mutation in a splice site of the DaSOD gene
   iii. a mutation in the DaSOD gene resulting in a frameshift mutation
   iv. a mutation resulting in a deletion of a part of the DaSOD gene,
   wherein the wild type DaSOD gene encodes a polypeptide of SEQ ID NO:4.
39. The method according to any one of the preceding items, wherein the yeast strain is of the species Dekkera anomalus, and said yeast strain comprises a mutant DaSOD gene encoding a mutant DaSOD protein lacking at least 50 amino acids, such as at least 70 amino acids, such as at least 100 amino acids, such as at least 150 amino acids of SEQ ID NO: 4.
40. The method according to any one of the preceding items, wherein the yeast strain is of the species Dekkera anomalus, and said yeast strain comprises a mutant DaSOD gene encoding a mutant DaSOD protein lacking at least the 50 most C-terminal amino acids, such as at least the 100 most C-terminal amino acids, such as at least the 150 most C-terminal amino acids of SEQ ID NO: 4.
41. The method according to any one of the preceding items, wherein the yeast strain is of the species Dekkera bruxellensis, and said yeast strain carries a mutation in the DbSOD gene resulting in a mutant DbSOD gene encoding a mutant DbSOD protein lacking one or more of the amino acids of SEQ ID NO:8.
42. The method according to any one of the preceding items, wherein the yeast strain is of the species Dekkera bruxellensis, and said yeast strain carries one or more of the following mutations
   i. a mutation introducing a premature stop codon in the DbSOD gene
   ii. a mutation in a splice site of the DbSOD gene
   iii. a mutation in the DbSOD gene resulting in a frameshift mutation
   iv. a mutation resulting in a deletion of a part of the DbSOD gene,
   wherein the wild type DbSOD gene encodes a polypeptide of SEQ ID NO:8.
43. The method according to any one of the preceding items, wherein the yeast strain is of the species Dekkera bruxellensis, and said yeast strain comprises a mutant DbSOD gene encoding a mutant DbSOD protein lacking at least 50 amino acids, such as at least 70 amino acids, such as at least 100 amino acids, such as at least 150 amino acids of SEQ ID NO: 8.
44. The method according to any one of the preceding items, wherein the yeast strain is of the species Dekkera bruxellensis, and said yeast strain comprises a mutant DbSOD gene encoding a mutant DbSOD protein lacking at least the 50 most C-terminal amino acids, such as at least the 100 most C-terminal amino acids, such as at least the 150 most C-terminal amino acids of SEQ ID NO: 8.
45. The method according to any one of the preceding items, wherein the yeast strain carries a SOD gene comprising a mutant SOD promoter.
46. The method according to any one of the preceding items, wherein the yeast strain carries a mutation in the SOD gene leading to loss of SOD function.
47. A method of producing a malt and/or cereal based beverage comprising less than 3% ethanol, said method comprising the steps of
   i) providing an aqueous extract of malt and/or cereal kernels
   ii) providing a Dekkera yeast strain, wherein said yeast strain is not capable of utilizing more than 2% maltose
   iii) fermenting said aqueous extract with said yeast
   thereby obtaining said malt and/or cereal based beverage.
48. A method of producing a malt and/or cereal based beverage comprising less than 3% ethanol, said method comprising the steps of
   i) providing an aqueous extract of malt and/or cereal kernels
   ii) providing a Dekkera yeast strain, wherein said yeast strain is not capable of utilizing more than 2% maltose when incubated at 25° C. for 10 days in an aqueous solution comprising in the range of 40 to 100 g/kg maltose and in the range of 8 to 50 g/kg glucose,
   iii) fermenting said aqueous extract with said yeast
   thereby obtaining said malt and/or cereal based beverage.
49. A method of producing a malt and/or cereal based beverage comprising less than 3% ethanol, said method comprising the steps of
   i) providing an aqueous extract of malt and/or cereal kernels
   ii) providing a Dekkera yeast strain, wherein said yeast strain is not capable of growing in an aqueous solution comprising maltose as a sole carbon source
   iii) fermenting said aqueous extract with said yeast
   thereby obtaining said malt and/or cereal based beverage.
50. The method according to any one of the preceding items, wherein the yeast strain is selected from the group consisting of Dekkera and Brettanomyces.
51. The method according to any one of the items 1 to 4 and 47 to 49, wherein the yeast strain is a Brettanomyces yeast strain.
52. The method according to any one of the items 1 to 4 and 47 to 49, wherein the yeast strain is selected from the group consisting of Brettanomyces nanus, Brettanomyces naardenensis, Brettanomyces custerisianus, Brettanomyces anomalus and Brettanomyces bruxellensis.
53. The method according to any one of the items 1 to 4 and 47 to 49, wherein the yeast strain is Dekkera bruxellensis and/or Dekkera anomalus.
54. The method according to any one of the items 1 to 4 and 47 to 49, wherein the yeast strain is Dekkera bruxellensis.
55. The method according to any one of the preceding items, wherein the fermentation of the aqueous extract is performed at a temperature in the range of 5 to 30° C., such as 10 to 25° C., such as 15 to 20° C.
56. The method according to any one of the preceding items, wherein the fermentation of the aqueous extract is in the range of 1 to 45 days, such as 1 to 21 days, such as 2 to 10 days, such as 3 to 7 days.
57. The method according to any one of the preceding items, wherein the aqueous extract is wort.
58. The method according to any one of the preceding items, wherein the aqueous extract is a fermented malt and/or cereal based beverage.
59. The method according to any one of the preceding items, wherein the aqueous extract is a beer.
60. The method according to any of items 1 to 58, wherein the malt and/or cereal based beverage is a low-alcohol malt and/or cereal based beverage.
61. The method according to any of the preceding items, wherein the malt and/or cereal based beverage is an alcohol-free malt and/or cereal based beverage.
62. The method according to any of the preceding items, wherein the malt and/or cereal based beverage is a beer, such as a low-alcohol beer or an alcohol-free beer.
63. The method according to any one of the preceding claims, wherein the malt and/or cereal based beverage comprises less than 2% ethanol, such as less than 1.5% ethanol, such as less than 1.0% ethanol, such as less than 0.5% ethanol, such as less than 0.3% ethanol, such as less than 0.1% ethanol.
64. The method according to any of the preceding items, wherein the malt and/or cereal based beverage has a p-citronellol concentration of less than 25 μg/L of, such as less than 20 μg/L.
65. The method according to any of the preceding items, wherein the malt and/or cereal based beverage has a geraniol concentration of at least 18 μg/L of, such as at least 20 μg/L.
66. The method according to any one of the preceding claims, wherein the aqueous extract contains more than 40 g/kg maltose, such as 40 to 100 g/kg maltose.
67. The method according to any of the preceding items, wherein the aqueous extract contains at the most 15 g/kg glucose, such as at the most 10 g/kg glucose, for example at the most 5 g/kg glucose.
68. The method according to any of the preceding items, wherein the aqueous solution contains 8 to 50 g/kg glucose
69. The method according to any one of the preceding items, wherein the method further comprises step(s) of processing said fermented aqueous extract into a beverage.
70. The method according to item 69, wherein the steps of processing comprise one or more of the following:
   iv. filtration
   v. optionally lagering
   vi. carbonation
   vii. bottling
71. The method according to any one or the preceding items, wherein the beverage is a beer.
72. The method according to any one of the preceding items, wherein the beverage is a low-alcohol beer.
73. The method according to any of the preceding items, wherein the beverage is a non-alcohol beer.
74. The method according to any of the preceding items, wherein the yeast strain is not capable of utilizing more than 2% of the maltose present in the aqueous extract, such as not more than 1.5% maltose, such as not more than 1% maltose.
75. The method according to any of the preceding items, wherein the yeast strain is not capable of utilizing more than 1% maltose.
76. The method according to any of the preceding items, wherein the yeast strain is not capable of utilizing any maltose.
77. The method according to any one of the preceding items, wherein said yeast strain is not capable of utilizing more than 2%, such as not more than 1.5%, for example not more than 1% maltose of the maltose in an aqueous extract, when incubated in said aqueous extract at 5 to 25° C. for 3 to 7 days, wherein said aqueous extract comprises glucose and maltose.
78. The method according to any one of the preceding items, wherein said yeast strain is not capable of utilizing more than 2% maltose when incubated at 25° C. for 10 days in an aqueous solution comprising in the range of 40 to 100 g/kg maltose and in the range of 8 to 50 g/kg glucose.
79. The method according to any one of the preceding items, wherein the yeast strain is not capable of utilizing maltose as sole carbon source.
80. The method according to any one of the preceding items, wherein the yeast strain is not capable of utilizing maltose as sole carbon source.
81. The method according to any one of the preceding items, wherein said yeast carries a mutation in or a deletion of one or more of the following genes:
   c. MTRA1, wherein the MTRA1 gene for example encodes a MTRA1 protein of SEQ ID NO:10 or 16 or a functional homolog thereof sharing at least 95% sequence identity therewith
   d. MTRA2, wherein the MTRA2 gene for example encodes a MTRA2 protein of SEQ ID NO:14 or 20 or a functional homolog thereof sharing at least 95% sequence identity therewith;
   e. ISOM(1), wherein the ISOM(1) gene for example encodes a ISOM(1) protein of SEQ ID NO:22 or a functional homolog thereof sharing at least 95% sequence identity therewith;
   f. ISOM, wherein the ISOM gene for example encodes a ISOM protein of SEQ ID NO:12 or a functional homolog thereof sharing at least 95% sequence identity therewith;
   g. ISOM(2) wherein the ISOM(2) gene for example encodes a ISOM(2) protein of SEQ ID NO:18 or a functional homolog thereof sharing at least 95% sequence identity therewith;
   h. MTRA3, wherein the MTRA3 gene for example encodes a MTRA3 protein of SEQ ID NO:26 or a functional homolog thereof sharing at least 95% sequence identity therewith;
   i. MTRA4, wherein the MTRA4 gene for example encodes a MTRA4 protein of SEQ ID NO:28 or a functional homolog thereof sharing at least 95% sequence identity therewith;
   j. MTRA5, wherein the MTRA5 gene for example encodes a ISOM protein of SEQ ID NO:30 or a functional homolog thereof sharing at least 95% sequence identity therewith;
   k. MTRA6, wherein the MTRA6 gene for example encodes a MTRA6 protein of SEQ ID NO:32 or a functional homolog thereof sharing at least 95% sequence identity therewith.
82. The method according to any one of the preceding items, wherein the yeast strain is a Dekkera bruxellensis yeast strain, said strain lacks the gene encoding DbMTRA1 of SEQ ID NO:16 or a functional homologue thereof having at least 98% sequence identity herewith.
83. The method according to any one of the preceding items, wherein the yeast strain is a Dekkera anomalus yeast strain, said strain lacks the gene encoding DaMTRA1 of SEQ ID NO:10 or a functional homologue thereof having at least 98% sequence identity herewith.
84. The method according to any one of the preceding items, wherein the yeast strain carries a mutation in the MTRA1 gene leading to a loss of MTRA1 function.
85. The method according to any one of the preceding items, wherein the yeast strain is a Dekkera bruxellensis yeast strain, wherein said yeast strain carries a mutation in the DbMTRA1 gene leading to a loss of DbMTRA1 function.
86. The method according to any one of the preceding items, wherein the yeast strain is a Dekkera anomalus yeast strain, said yeast strain carries a mutation in the DaMTRA1 gene leading to a loss of DaMTRA1 function.
87. The method according to any one of the preceding items, wherein the yeast strain is a Dekkera bruxellensis yeast strain, said yeast strain carries one or more mutation(s) resulting in a mutant DbMTRA1 gene encoding a mutant DbMTRA1 protein comprising one or more amino acid substitutions, such as 4 or more, such as 8 or more, such as 12 or more, such as 14 or more amino acid substitutions in the N-terminal region consisting of amino acids 1 to 65 of DbMTRA1 of SEQ ID NO: 16.
88. The method according to any one of the preceding items, wherein the yeast strain is a Dekkera anomalus yeast strain, said yeast carries one or more mutation(s) resulting in a mutant DaMTRA1 gene encoding a mutant DaMTRA1 protein comprising one or more amino acid substitutions, such as 4 or more, such as 8 or more, such as 12 or more, such as 14 or more amino acid substitutions in the N-terminal region consisting of amino acids 1 to 65 of DaMTRA1 of SEQ ID NO: 10.
89. The method according to any one of the preceding items, wherein the yeast strain is a Dekkera bruxellensis yeast strain, said yeast strain carries a mutation resulting in a mutant DbMTRA1 gene encoding a mutant DbMTRA1 protein lacking one or more amino acid, such as lacking at least 4 amino acids, such as lacking at least 8, such as lacking at least 12, such as lacking at least 14 amino acids of SEQ ID NO:16.
90. The method according to any one of the preceding items, wherein the yeast strain is a Dekkera anomalus yeast strain, said yeast strain carries a mutation resulting in a mutant DaMTRA1 gene encoding a mutant DaMTRA1 protein lacking one or more amino acid, such as lacking at least 4 amino acids, such as lacking at least 8, such as lacking at least 12, such as lacking at least 14 amino acids of SEQ ID NO:10.
91. The method according to any one of the preceding items, wherein the yeast carries a mutation in the MTRA1 gene, wherein the mutation is:
   a mutation resulting in a frameshift mutation;
   a mutation resulting in formation of a premature stop codon in the MTRA1 gene;
   a mutation in a splice site of the MTRA1 gene;
   a mutation in the promoter region of the MTRA1 gene; and/or
   a mutation in the an intron of the MTRA1 gene
92. The method according to any one of the preceding items, wherein the yeast is a Dekkera bruxellensis yeast strain, said yeast strain carries a mutation in the DbMTRA1 gene of SEQ ID NO:15, wherein the mutation is:
   a mutation resulting in a frameshift mutation;
   a mutation resulting in formation of a premature stop codon in the DbMTRA1 gene;
   a mutation in a splice site of the DbMTRA1 gene;
   a mutation in the promoter region of the DbMTRA1 gene; and/or
   a mutation in the an intron of the DbMTRA1 gene.
93. The method according to any one of the preceding items, wherein the yeast is a Dekkera anomalus yeast strain, said yeast strain carries a mutation in the DaMTRA1 gene of SEQ ID NO:9, wherein the mutation is:
   a mutation resulting in a frameshift mutation;
   a mutation resulting in formation of a premature stop codon in the DaMTRA1 gene;
   a mutation in a splice site of the DaMTRA1 gene;
   a mutation in the promoter region of the DaMTRA1 gene; and/or
   a mutation in the an intron of the DaMTRA1 gene.
94. The method according to any one of the preceding items, wherein the yeast strain is a Dekkera bruxellensis yeast strain, said yeast strain comprising a mutation in or a deletion of the gene encoding DbISOM(2) of SEQ ID NO:18 or a functional homologue thereof having at least 98% sequence identity herewith.
95. The method according to any one of the preceding items, wherein the yeast strain is a Dekkera anomalus yeast strain, said yeast strain comprising a mutation in or a deletion of the gene encoding DaISOM of SEQ ID NO:12 or a functional homologue thereof having at least 98% sequence identity herewith.
96. The method according to any one of the preceding items, wherein the yeast strain carries one or more mutation(s) in one or more of the ISOM gene(s) leading to a loss of function for one or more of the ISOM(s).
97. The method according to any one of the preceding items, wherein the yeast strain is a Dekkera bruxellensis yeast strain, said yeast strain carries one or more mutation(s) in the DbISOM(2) gene leading to a loss of DbISOM(2) function.
98. The method according to any one of the preceding items, wherein the yeast strain is a Dekkera anomalus yeast strain, said yeast strain carries one or more mutation(s) in the DaISOM(2) gene leading to a loss of DaISOM(2) function.
99. The method according to any one of the preceding items, wherein the yeast strain carries a carries a frameshift mutation in one or more of the ISOM genes resulting in a truncation of one or more of the ISOM proteins.
100. The method according to any one of the preceding items, wherein the yeast strain is a Dekkera bruxellensis yeast strain, said yeast strain carries a carries a frameshift mutation in the DbISOM(2) gene resulting in a truncation of the DbISOM(2) protein.
101. The method according to any one of the preceding items, wherein the yeast strain is a Dekkera anomalus yeast strain, said yeast strain carries a carries a frameshift mutation in the DaISOM gene resulting in a truncation of the DaISOM protein.
102. The method according to any one of the preceding items, wherein the yeast carries a mutation in one or more of the ISOM genes, wherein the mutation is:
   a mutation resulting in a frameshift mutation;
   a mutation resulting in one or more amino acid substitution in one or more ISOM(s);
   a mutation resulting in formation of a premature stop codon in one or more ISOM genes;
   a mutation in a splice site of one or more ISOM genes;
   a mutation in the promoter region of one or more ISOM genes; and/or
   a mutation in the an intron of one or more of the ISOM genes.
103. The method according to any one of the preceding items, wherein said yeast strain is a Dekkera bruxellensis yeast strain, said yeast strain carries a mutation in the DbISOM(2) gene of SEQ ID NO:17, wherein the mutation is:
   a mutation resulting in a frameshift mutation;
   a mutation resulting in one or more amino acid substitution of DbISOM(2);
   a mutation resulting in formation of a premature stop codon in the DbISOM(2) gene;
   a mutation in a splice site in the DbISOM(2) gene;
   a mutation in the promoter region of the DbISOM(2) gene; and/or
   a mutation in an intron of the DbISOM(2) gene.
104. The method according to any one of the preceding items, wherein the yeast is a Dekkera anomalus yeast strain said yeast strain carries a mutation in the DaISOM gene of SEQ ID NO: 11, wherein the mutation is:
   a mutation resulting in a frameshift mutation;
   a mutation resulting in one or more amino acid substitution of DaISOM;
   a mutation resulting in formation of a premature stop codon in the DaISOM gene;
   a mutation in a splice site in the DaISOM gene;
   a mutation in the promoter region of the DaISOM gene; and/or
   a mutation in an intron of the DaISOM gene.
105. The method according to any one of the preceding items, wherein the yeast strain is a Dekkera bruxellensis yeast strain, said yeast strain carries a frameshift mutation, a mutation resulting in formation of a premature stop codon or a splice mutation resulting in a mutant DbSOM(2) gene encoding a mutant DbISOM(2) protein lacking at least the 50 most C-terminal amino acids, such as lacking at least the 100 most C-terminal amino acids, such as at least the 150 most C-terminal amino acids, such as at least the 200 most C-terminal amino acids of SEQ ID NO:18.
106. The method according to any one of the preceding items, wherein the yeast strain is a Dekkera bruxellensis yeast strain, said yeast comprising a mutation in or a deletion of the gene encoding DbMTRA2 of SEQ ID NO:20 or a functional homologue thereof having at least 98% sequence identity herewith.
107. The method according to any one of the preceding items, wherein the yeast strain is a Dekkera anomalus yeast strain, said yeast strain comprising a mutation in or a deletion of the gene encoding DsMTRA2 of SEQ ID NO:14 or a functional homologue thereof having at least 98% sequence identity herewith.
108. The method according to any one of the preceding items, wherein the yeast strain is a Dekkera bruxellensis yeast strain, said yeast strain comprising a mutation in or a deletion of the gene encoding DbMTRA3 of SEQ ID NO:26 or a functional homologue thereof having at least 98% sequence identity herewith.
109. The method according to any one of the preceding items, wherein the yeast strain is a Dekkera bruxellensis yeast strain, said yeast comprising a mutation in or a deletion of the gene encoding DbMTRA4 of SEQ ID NO:28 or a functional homologue thereof having at least 98% sequence identity herewith.
110. The method according to any one of the preceding items, wherein the yeast strain is a Dekkera bruxellensis yeast strain, said yeast strain comprising a mutation in or a deletion of the gene encoding DbMTRA5 of SEQ ID NO:30 or a functional homologue thereof having at least 98% sequence identity herewith.
111. The method according to any one of the preceding items, wherein the yeast strain is a Dekkera bruxellensis yeast strain, said yeast strain comprising a mutation in or a deletion of the gene encoding DbMTRA6 of SEQ ID NO:32 or a functional homologue thereof having at least 98% sequence identity herewith.
112. The method according to any one of the preceding items, wherein the yeast strain is a Dekkera bruxellensis yeast strain, said yeast comprising a mutation in or a deletion of the gene encoding DbISOM(1) of SEQ ID NO:22 or a functional homologue thereof having at least 98% sequence identity herewith.
113. The method according to any one of the preceding items, wherein the yeast strain further is not capable of utilizing more than 5% maltotriose.
114. The method according to any one of the preceding items, wherein the yeast strain further is not capable of utilizing more than 5% maltotetraose.
115. The method according any one of the preceding items, wherein the yeast strain is capable of utilizing glucose.
116. The method according to any one of the preceding items, wherein the yeast strain is not capable of generate more than 1.5 promille ethanol per °Plato, such as 1.4 promille ethanol per °Plato, such as 1.1 promille ethanol per °Plato.
117. A Dekkera yeast strain, wherein said yeast strain is not capable of converting more than 25% of p-coumaric acid into 4-ethylphenol when incubated in an aqueous solution comprising p-coumaric acid.
118. A Dekkera yeast strain carrying a mutation in or a deletion of one or more of the following genes:
   a. PAD;
   b. SOD.
119. The yeast strain according to any one of items 117 to 118, wherein the yeast strain is a Dekkera anomalus yeast strain carrying a mutation in or a deletion of one or more the following genes:
   i. the DaPAD1 gene encoding DaPAD1 of SEQ ID NO:2 or a functional homologue thereof having at least 80%, such as at least 90%, for example at least 95% sequence identity herewith;
   ii. the DaSOD gene encoding DaSOD of SEQ ID NO:4 or a functional homologue thereof having at least 80%, such as at least 90%, for example at least 95% sequence identity herewith.
120. The yeast strain according to any one of items 117 to 119, wherein the yeast strain further carries a mutation in or a deletion of one or more the following genes:
   i. MTRA1, wherein the MTRA1 gene encodes a MTRA1 protein of SEQ ID NO:10 or 16 or a functional homolog thereof sharing at least 95% sequence identity therewith
   ii. MTRA2, wherein the MTRA2 gene encodes a MTRA2 protein of SEQ ID NO:14 or 20 or a functional homolog thereof sharing at least 95% sequence identity therewith;
   iii. ISOM, wherein the ISOM gene encodes a ISOM protein of SEQ ID NO:12 or a functional homolog thereof sharing at least 95% sequence identity therewith.
121. The yeast strain according to any one of items 117 to 120, wherein the yeast strain is a Dekkera bruxellensis yeast strain carrying a mutation in or a deletion of one or more the following genes:
   i. the DbPAD2 gene encoding DbPAD2 of SEQ ID NO:6 or a functional homologue thereof having at least 80%, such as at least 90%, for example at least 95% sequence identity herewith;
   ii. the DbSOD gene encoding DbSOD of SEQ ID NO:8 or a functional homologue thereof having at least 80%, such as at least 90%, for example at least 95% sequence identity herewith.
122. The yeast strain according to any one of items 117 to 121, wherein the yeast strain is as defined in any one of items 1 to 46.
123. A Dekkera yeast strain carrying one or more of the mutations and/or deletions identified in any one of items 81 to 112.
124. A beverage prepared by the method according to any one of items 1 to 116.

### EXAMPLES

The invention is further illustrated by the following examples, which should however not be construed as limiting for the invention.

### Example 1

Ferulic Acid Uptake—in YPD Medium Supplemented with Ferulic Acid

Phenolic off-flavor production was studied among yeast strains selected from Brettanomyces custersianus, Brettanomyces naardensis, Dekkera, bruxellensis, Dekkera anomalus, using an absorbance-based method based on the uptake of ferulic acid.

The yeast strain cultures were diluted 1:100 in water before inoculation in triplicates in YPD supplemented with ferulic acid at 0.1 mg/mL. The yeast strains were grown until late exponential phase. After 1 week cultivation at 25° C. with agitation, plates were centrifuged (4000 rpm, 5 min, 4° C.), 100 μl of supernatant was collected and absorbance was measured at 325 nm in a Spark® Multimode Plate Reader (TECAN).

The results show that both B. custersianus and B. naardensis species are not able to convert ferulic acid into secondary metabolites. Only one B. naardensis strain seemed to convert ferulic acid to some extend. Most strains of D. anomalus and D. bruxellensis were able to convert ferulic acid. Surprisingly, one strain belonging to D. anomalus species (CRL-90) was not capable of converting ferulic acid.

### Example 2

Conversion of Ferulic Acid into 4-Ethylguaiacol and p-Coumaric Acid into 4-Ethylphenol in Wort

Strains were propagated in pilsner wort, in 50 mL Erlenmeyer shake flasks. A pitching rate of 100,000 cells/mL was determined using a Cellometer X2 (Nexelom Bioscience) to count the cells. Fermentations were performed in duplicates in 250 ml Duran bottles containing 200 ml of standard pilsner wort (Viking Malt). Cumulative pressure was monitored with ANKOM RF Gas Production System® (ANKOM). Fermentations were stopped after 7 days, cells were removed by centrifugation (4,000 g, 10 min, 4° C.) and supernatant was used for analysis.

Phenolic compounds (ferulic acid, coumaric acid, 4-ethylguaiacol, 4-ethylphenol) were quantified by Ultra Performance Liquid Chromatography (UPLC) (Waters) with PDA detection (280 nm). Separation was achieved using the BEH Phenyl Ultra Column (2.1×100 mm, 1.7 um) and a flow of 0.5 ml/min. Injection volume was 1 ul. The mobile phase was 99.9% A, 0.1% B between 0 and 3 min. followed by a gradient up to 45% B during 5 min. Eluent A was contained 3% Formic acid, 10% methanol in water and Eluent B was 100% methanol. Calibration standards were prepared in methanol in the range 0.1-10 mg/l by dilution of a 10 mg/l standard mix. Beer samples were filtered on a 0.2 um filter, diluted 1.5× with eluent A. and vortexed for 5 sec. Compounds were identified by retention time and ID was confirmed by spiking with standard solution.

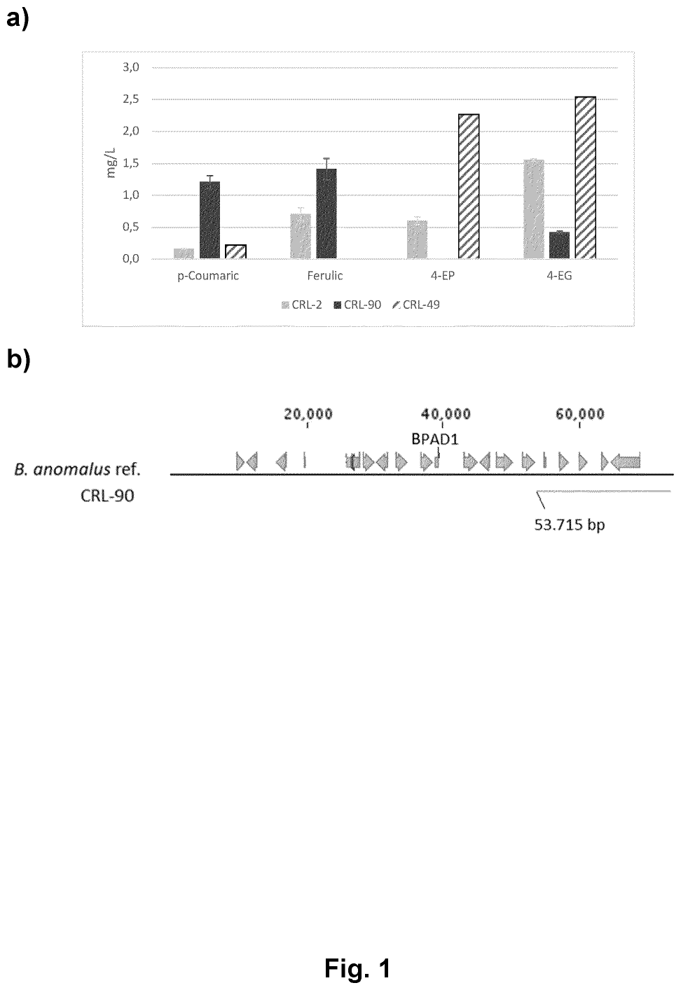

The content of volatile phenols in the beer produced was is included in FIG. 1A.

While both 4-ethylphenol and 4-ethylguaiacol were detected in the control strain CRL-2 and CRL-49, 4-ethylphenol was absent and a minimum amount of 4-ethylguaiacol was quantified after fermentation with CRL-90 (Dekkera anomalus). Intermediates 4-vinylphenol and 4-vinylguaiacol were below threshold in all fermentations. These results indicate that CRL-90 is not able to convert p-coumaric acid into 4-ethylphenol and had a reduced ability to convert ferulic acid into 4-ethylguaiacol. Thus, CRL-90 is the first Dekkera without POF identified.

### Example 3

### Genomic Data

The results of lacking the ability to convert ferulic acid into 4-ethylguaiacol and p-coumaric acid into 4-ethylphenol, are supported by genomic data.

When the full scaffold of Dekkera anomalus, CRL-90 was compared with BLAST to a D. anomalus reference genome (CRL-49), it was found that the yeast strain CRL-90 was missing the N-terminal part comprising the first 1-53,714 bp (FIG. 1B). It was further found that the missing region contained the DaPAD1 gene in the reference strain.

Thus, the CRL-90 strain is lacking the DaPAD1 gene.

The putative decarboxylase encoded by the gene DaPAD1 in Dekkera shares limited sequence identity with the decarboxylase known from other yeast species. One example hereof is that the decarboxylase in Saccharomyces cerevisiae, named FDC1, only shares 8.12% and 9.50% amino acid sequence identity with the putative decarboxylase in Dekkera anomalus (DaPAD1). In Saccharomyces cerevisiae, ScFDC1 is activated by ScPAD1. DaPAD1 shares 12.85% amino acid sequence identity with ScPAD1, however their function seems to differ, as the putative function of DaPAD1 is a decarboxylase activity and ScPAD1 acts as an activator of ScFDC1. See Table 1 below.

Interestingly, the amino acid sequence identity between the two Dekkera species differs as well. DaPAD1 shares 68.64% and 85.31% sequence identity with DbPAD2 and DbPAD1 respectively. See Table 1 below.

TABLE 1
Amino acid sequence comparison. Top right part shows % identity.
Bottom left part shows number of differences (gaps and mismatches)

| | | 1 | 2 | 3 | 4 | 5 |
|---|---|---|---|---|---|---|
| B. bruxellensis PAD2 | 1 | | 80.45 | 85.31 | 14.06 | 8.32 |
| B. bruxellensis PAD1 | 2 | 43 | | 68.64 | 13.55 | 9.50 |
| B. anomalus PAD1 | 3 | 26 | 69 | | 12.85 | 8.12 |
| S. cerevisiae ScPAD1 | 4 | 214 | 236 | 217 | | 7.33 |
| S. cerevisiae ScFDC1 | 5 | 463 | 457 | 464 | 468 | |

When blasting the whole scaffold with a reference scaffold, the closest hit is shown below in Table 2 supporting that CRL-90 is lacking the DaPAD1 gene.

TABLE 2

| Parameter | BLAST scores of closest hit |
|---|---|
| Total score | 121,278 |
| Max score | 83,996 |
| Identities | 42,141 |
| Max % identity | 99,78 |
| HSP length | 42,229 |

### Example 4

### Maltose or Glucose Utilization as a Sole Carbon Source

Below we describe a test showing whether a yeast strain is capable of utilizing maltose or glucose as a sole carbon source.

Six Dekkera strains with different genomic maps were used. Four of the strains were Dekkera bruxellensis (CRL-1, CRL-2, CRL-19 and CRL-50), while two of them (CRL-49 and CRL-90) were Dekkera anomalus.

### YNB Media

Media consisting of Yeast Nitrogen Base with amino acids supplemented with 1% (corresponding to 10 g/L) glucose or maltose respectively as a sole carbon source were used to test the metabolic activity of the yeast cells, and hence indirectly to test the ability of the yeast cells to growth. The Dekkera strains were incubated in triplicates in Biolog® 96-well plates (Omnilog) at 25° C. without agitation, and growth kinetics was monitored with OmniLog® Biolog. The quantification was based on adding tetrazolium dye that is reduced to purple formazan dependent on NADH production, which can be used as a surrogate measure of strain metabolic activity. Strain growth can frequently be correlated to metabolic activity and thus growth can frequently be determined based on generation of purple color.

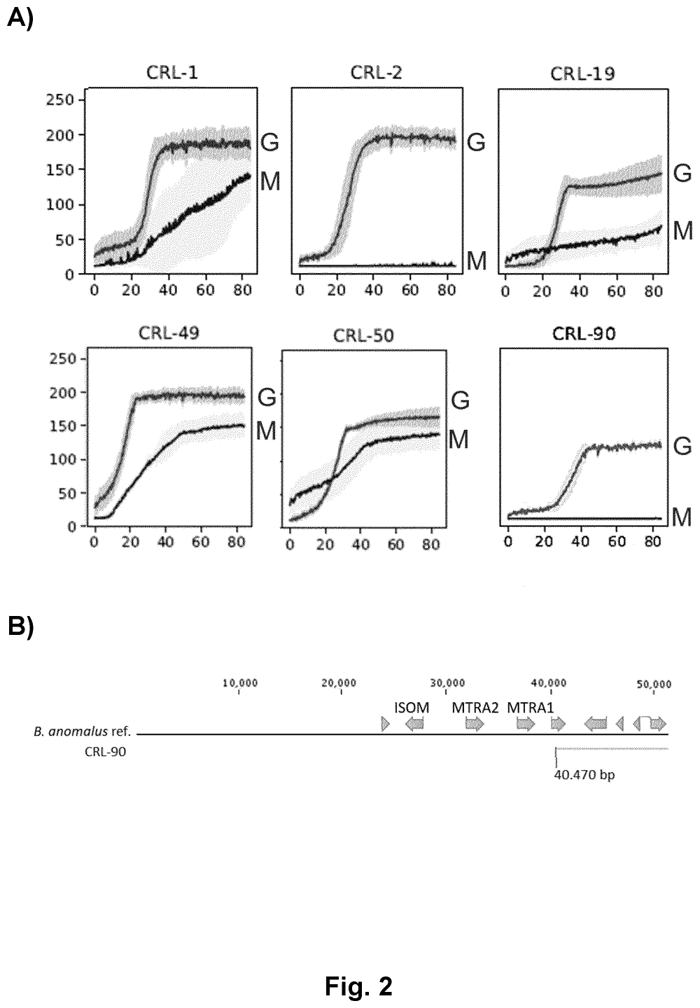

To test the ability of the yeast to utilize maltose or glucose, the yeast was grown for 85 hours in synthetic media (see FIG. 2A). The x-axis shows the time in hours and the y-axis the quantification of metabolic activity based on color change.

As can be seen from FIG. 2, CRL-1, CRL-19, CRL-49 and CRL-50 were able to utilize both glucose (G) and maltose (M). However, CRL-19 was not able to utilize maltose (M) to the same extend as CRL-1, CRL-49 and CRL50. CRL-2 and CRL-90 were only able to utilize glucose (G) but not maltose (M). Thus, CRL-2 and CRL-90 both showed insignificant metabolic activity when incubated with maltose as sole carbon source.

### Example 5

### Capability of Utilizing Different Fermentable Sugars in Wort

Below we describe a test showing whether a yeast strain is capable of utilizing different fermentable sugars, such as glucose, maltose, maltotriose, or maltotetraose in wort.

### Wort as a Yeast Media

### Wort 1

In order to investigate the ability of Dekkera to utilize fermentable sugars in wort, an all malt pale wort, 16°Plato was used for primary fermentation with the following strains: CRL-1, CRL-2, CRL-19, CRL-49 and CRL-50. Fermentation was performed at 25° C. for 10 days.

CRL-1, CRL-2, CRL-19 and CRL-50 are Dekkera bruxellensis and CRL-49 is Dekkera anomalus.

Fermentable sugars were quantified with High Performance Liquid Chromatography (HPLC) using a DIONEX column. Ethanol content was obtained with Alcolyser BeerME Analyzing System (www.anton-paar.com). The results are shown in the table 3 below:

TABLE 3

| | Ethanol | Glucose | Maltose | Maltotriose | Maltotetraose |
|---|---|---|---|---|---|
| | (% v/v) | (mg/kg) | (mg/kg) | (mg/kg) | (mg/kg) |
| Wort | 0 | 9835.98 | 53598.12 | 13846.41 | 2614.66 |
| CRL-1 | 7.49 ± 0.13 | 4 ± 2 | 0 ± 0 | 179 ± 19 | 3225 ± 150 |
| CRL-2 | 1.71 ± 0.02 | 91 ± 16 | 60215 ± 3559 | 16674 ± 839 | 3325 ± 157 |
| CRL-19 | 7.39 ± 0.08 | 10 ± 14 | 22 ± 30 | 67 ± 46 | 2686 ± 1072 |
| CRL-49 | 7.3 ± 0.05 | 0 ± 0 | 15 ± 21 | 3403 ± 388 | 3064 ± 148 |
| CRL-50 | 7.75 ± 0.32 | 37 ± 4 | 1570 ± 549 | 19 ± 7 | 81 ± 1 |

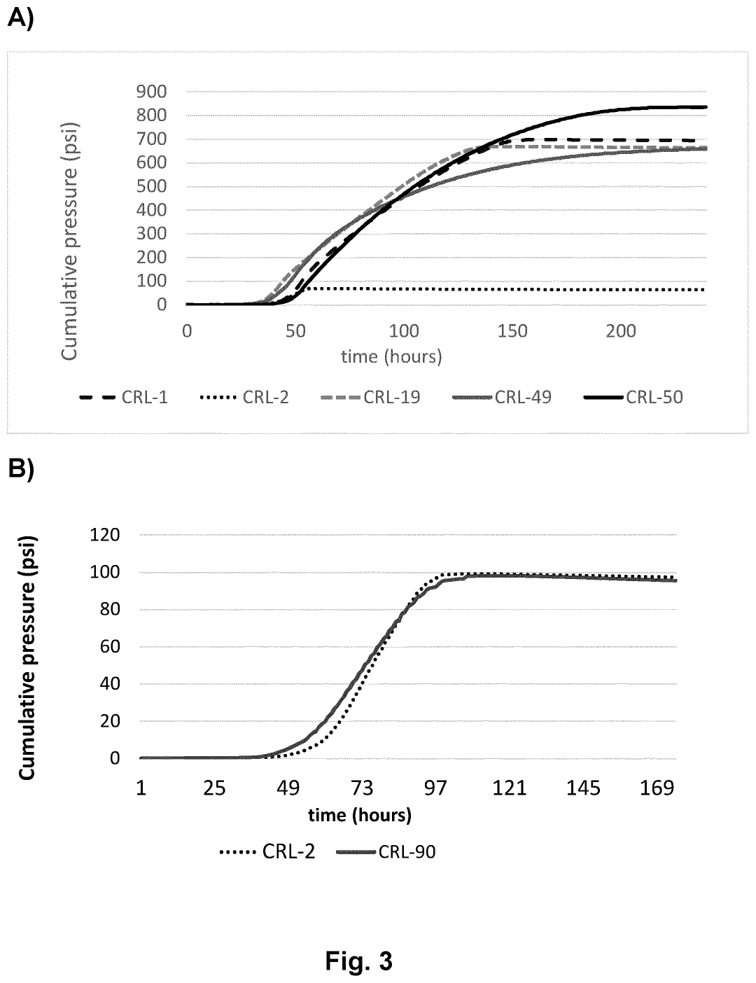

The fermentation for all strains proceeded in a similar way, as shown by CO₂ accumulation (FIG. 3A) and ethanol produced (7.5±0.2%; Table 3), except for CRL-2 which was not able to metabolize maltose. CRL-2 produced 1 0.71±0.02% v/v ethanol.

### Wort 2

A lager beer wort prepared from malt and sugar (70/30 malt to sugar) was used for primary fermentation with the following strain CRL-90 in 200 mL. CRL-90 is Dekkera anomalus. Fermentation was performed at 25° C. for 10 days.

Fermentable sugars were quantified with High Performance Liquid Chromatography (HPLC) as described above. The results are shown in the table 4 below:

TABLE 4

| QA_HPLC | Wort “70/30” | Dekkera “70/30” |
|---|---|---|
| Fructose, mg/kg | 2416.17 | 43.98 |
| Glucose, mg/kg | 19610.30 | 124.07 |
| Isomaltose, mg/kg | | 111.81 |
| Isomaltotriose, mg/kg | | 124.9 |
| Maltoheptaose, mg/kg | | 80.83 |
| Maltohexaose, mg/kg | | 347.96 |
| Maltooctaose, mg/kg | | 0 |
| Maltopentaose, mg/kg | | 713.97 |
| Maltose, mg/kg | 60840.05 | 64974.43 |
| Maltotetraose, mg/kg | | 1275.96 |
| Maltotriose, mg/kg | 9609.67 | 9963.13 |
| Panose, mg/kg | | 449.93 |
| Sucrose, mg/kg | 3498.56 | 3291.56 |
| Ethanol, % v/v | | 1.39 |

The fermentation for CRL-90 proceeded in a similar way as for CRL-2, as shown by CO₂ accumulation (FIG. 2B). CRL-90 was not able to utilize any of the maltose present in the 70/30 wort. CRL-90 produced 1.39% v/v ethanol.

### Example 6

### Putative Maltose Assimilation Genes

The yeast strains were grown for one week in 200 ml in Yeast Peptone Dextrose (YPD) yeast extract (1%) peptone (2%) dextrose (2%) at 25° C. with agitation. Cells were collected by centrifugation at 4000 g, 4° C., washed by suspension in water and collected in the same conditions.

As seen from Table 4, CRL-90 was not able to utilize any maltose.

When the full scaffold of Dekkera anomalus, CRL-90, was compared with BLAST to a Dekkera anomalus reference genome, CRL-49 it was found that strain CRL-90 was missing the N-terminal part comprising 1-40,469 bp see FIG. 2B. It was further found that the missing region comprised a maltose assimilation cluster comprising DaMTRA1, DaISOM and DaMTRA2 in the reference strain.

The genomes of CRL-1, CRL-2, CRL-19 and CRL-50 were sequenced with Single Molecule Real Time Technology (Pacific Biosciences). Good quality genomes were obtained for all samples. The genes identified for putative maltose assimilation were identified and compared. A BLAST search was used to find the specific proteins in each genome. Copy number for each protein was predicted based on the hits, filtering with % identity (>98%) and HSP length (full coverage). The results are shown in table 5 below.

TABLE 5

| Gene copies | DbMTRA2 | DbMAL12 | DbMTRA1 | DblSOM(1) | DblSOM(2) | DbMTRA5 |
|---|---|---|---|---|---|---|
| CRL-1 | 3 | 0 | 4 | 1 | 1 | 2 |
| CRL-2 | 2 | 1 | 1* | 1 | 1** | 2 |
| CRL-19 | 1 | 1 | 1 | 1 | 1 | 2 |
| CRL-50 | 1 | 0 | 3 | 1 | 1 | 2 |

*CRL-2 contains one copy number of DbMTRA1. The nucleotide sequence encoding the DbMTRA1 gene in CRL-2 shares 97.51% sequence identity with the nucleotide sequence encoding the DbMTRA1 gene in CRL-1, i.e. 44 nucleotides differ. The amino acid sequence identity between the CRL-2 DbMTRA1 protein and CRL-1 DbMTRA1 protein is 97.62%, i.e. 14 amino acids differ.
**CRL-2 lacks a gene encoding a functional DblOSM(2). CRL-2 carries a deletion at 1050 bp, which truncates the whole translation.

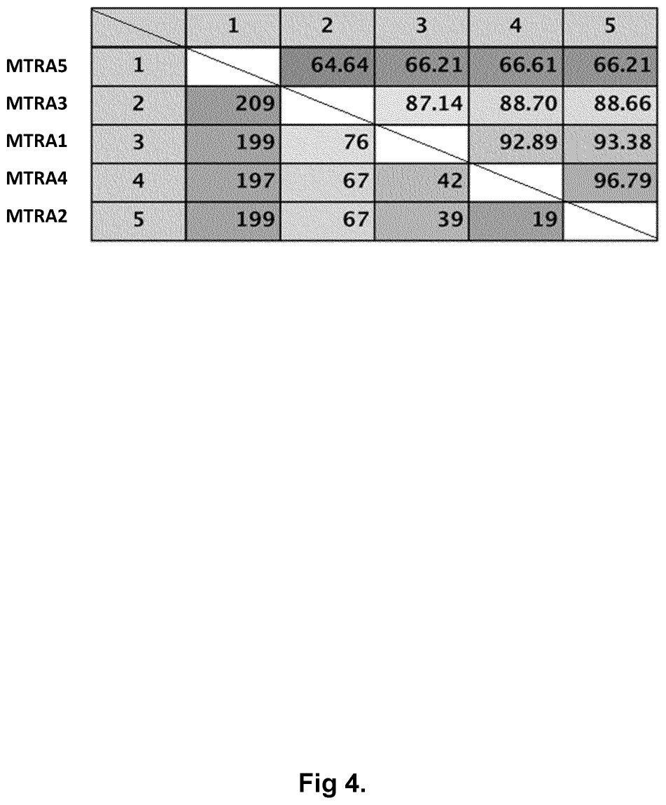

A nucleotide alignment of the DbMTRA1 gene sequences for CRL-1 (4 copies found), CRL-50 (3 copies found), CRL-19 (1 copy found), CRL-2 (1 copy found with 97.51% homology) is shown in FIG. 4A. The alignment displays the N-terminal nucleotide sequence of the DbMTRA1 maltose transporter. It was found that the CRL-2 copy has a completely different N-terminal sequence compared to CRL-1, CRL-19 and CRL-50.

An amino acid sequence alignment of all the copies found in DbMTRA1 was performed. Again, it can be concluded that the amino acid sequence of the N-terminal of CRL-2 DbMTRA1 protein is different from the amino acid sequence of DbMTRA1 protein of CRL-1, CRL-19 and CRL-50.

The putative maltose transporters encoded by the genes in Dekkera share limited sequence identity with maltose transporters known from other yeast species. One example hereof is that the maltose transporter, ScMAL31, in Saccharomyces cerevisiae shares approximately 47% sequence identity with the maltose transporter, DbMTRA1, found in Dekkera bruxellensis. This is also the case for the major isomaltases. The major isomaltases, ScIMA1, in Saccharomyces cerevisiae, shares only approximately 60% sequence identity with the putative major isomaltase, DbISOM, found in Dekkera bruxellensis.

### Example 7

### Beta-Glucosidase Activity and Flavor Production

Dekkera can contain two open reading frames (ORFs), which putatively encode for two beta-glucosidases, however the impact of the presence of these genes during beer brewing in Dekkera has not been explored previously.

### DNA Sequencing and Bioinformatics Analysis

The Dekkera yeast strains were grown in 100 mL Erlenmeyer flasks containing 50 mL of YPD, under aerobic conditions at 25° C. with agitation (100 rpm) for one week.

Cells were collected by centrifugation at 4000 g, 4° C., washed by suspension in water and collected in the same conditions. Samples were sent for DNA extraction and whole-genome sequencing, short insert PE150 library, on Illumina HiSeq4000 (BGI-Tech Solutions, Hong Kong). CLC Genomics Workbench software (www.qiagenbioinformatics.com) was used as a tool for bioinformatic analysis. Genome assembly of cells was performed in CLC software using De novo assembly feature. Genes of interest were found on GenBank, accession number (AKS48905.1, EIF45415.1, AKS48904.1) for DbBGL1, DbBGL2 and DaBGL Nucleotide BLAST tool on CLC software was used to identify the presence or absence of each gene.

See the results of the DbBGL1, DbBGL2 and DaBGL in table 6 below:

TABLE 6

| | | Beta-glucosidase ORFs | | | |
|---|---|---|---|---|---|
| Strain | Species | DbBGL1 | DbBGL2 | DaBGL | |
| CRL-1 | D. bruxellensis | X | — | — | [symbol] |
| CRL-2 | D. bruxellensis | — | X | — | |
| CRL-19 | D. bruxellensis | X | X | — | |
| CRL-27 | D. bruxellensis | X | — | — | |
| CRL-49 | D. anomalus | — | — | X | |
| CRL-50 | D. bruxellensis | — | — | — | |

Dekkera bruxellensis yeast strains, CR-1 and CRL-27 were found to have one DbBGL open reading frame (ORF), i.e. DbBGL1. CRL-2 had one DbBGL ORF, i.e. DbBGL2. CRL-19 had both ORFs, i.e. both DbBGL1 and DbBGL2. CRL-50 did not have any of the DbBGL OFRs. The Dekkera anomalus yeast strain, CRL-49, contained one OFR for DaBGL. [symbol]
indicates data missing or illegible when filed

To test beta-glucosidase activity in Dekkera, cells of interest were grown for one week in yeast peptone cellobiose (2%) (YPC) media. Extracellular, cell-associated and intracellular cell fractions were prepared in a method modified from Daenen et al. 2008. For the extracellular fraction, 1 ml of culture was transferred to a 1.5 ml Eppendorf tube (ThermoFisher), centrifuged (4,000 g, 5 min, 4° C.) and the supernatant was collected. Then, all the cultures were adjusted to give an optical density (OD) at 600 nm of 1. The cells were washed with sterile water and resuspended in phosphate buffered saline (PBS) buffer to collect the cell-associated enzyme fraction. In order to get the intracellular fraction, 0.5 mg/ml zymolyase (ThermoFisher) was added with PBS and incubated for 1 hour at 37° C. Then glass beads (425-600 μm, Sigma) were added to the cell fractions and vortexed for 20 seconds twice and kept on ice when not vortexed. Subsequently the suspension was spun down (14,000 g 10 min) and the supernatant was collected to give the intracellular fraction. The beta-glucosidase conversion in each fraction was determined with the MAK129 β-glucosidase assay kit (Sigma Aldrich). p-nitrophenyl-β-D-glucopyranoside (β-NPG) was used as the substrate and the extent of the reaction was measured at 405 nm after a 20 minutes incubation at 37° C. The assay was performed in a 96-well plate. The results are given in units/L, where one unit is the amount of enzyme that catalyzes the hydrolysis of 1.0 μmole of substrate per minute at pH=7 and 37° C.

Intracellular, cell-related and extracellular beta-glucosidase activity were measured in CRL-1, CRL-2, CRL19, CRL-49 and CRL-50. The greatest conversion in D. bruxellensis (up to 74 units/L) was detected in the intracellular fraction of CRL-19, which contains both beta-glucosidase ORFs. In contrast, very little substrate conversion was detected in the cells with only one or no beta-glucosidase encoding genes.

The results indicate that DbBGL2 is more efficient than DbBGL1 and suggest that there could be some kind of additive effect between the two proteins. The intracellular fraction of D. anomalus CRL-49 showed the highest activity among all the Brettanomyces strains tested (144 units/L).

### Flavor Production

In order to investigate the ability of Dekkera to aid in the release of hop aromas, two independent experimental set-ups were performed:

1) An all malt pale wort, 16 Plato was provided by Jacobsen Breweries for primary fermentation,

2) Jacobsen Indian Pale Ale (IPA) beer was also provided by Jacobsen Breweries and used for secondary fermentation, with extra 1,2% glucose added to favor growth.

Fermentations were performed using strains CRL-1, CRL-2, CRL-19, CRL-49 and CRL-50.

Strains were propagated in the above-mentioned CRL pilsner wort in 50MI Erlenmeyer flasks until the desired cell count was achieved. All fermentations were done in duplicates, performed in a 250 ml Duran bottles containing 200 ml of media. The fermentation was allowed to become anaerobic and the ANKOM RF Gas Production System (ANKOM) was used to monitor fermentation performance and CO₂ release. A pitching rate of 100,000 viable cells/mL was used, determined counting cells from the inoculum with a Cellometer X2 (Nexelom Bioscience). No samples were taking during fermentation in order to stop ingress of air. Beer was harvested when no more CO₂ release could be measured and then frozen at −20° C. before analysis.

Samples taken at the end of fermentation were analysed for monoterpene alcohols and compared to the starting wort. The results show that strains CRL-1 (one DbBGL ORF) and CRL-50 (no DbBGL ORFs), which had the lowest beta-glucosidase activities led to the greatest concentrations of β-citronellol, reaching levels up to 31.5 μg/L after fermentation for CRL-50. Furthermore, CRL-2 (lacking one ORF and being unable to utilize maltose) had the lowest conversion of geraniol to β-citronellol. The general pattern was seen in all the strains; the content of geraniol decreased in favor of production of p-citronellol. Linalool was converted to α-terpineol but at lower rates. Following conventional pathways, myrcene was completely depleted in all cases and isoamyl isobutyrate was slightly increased.

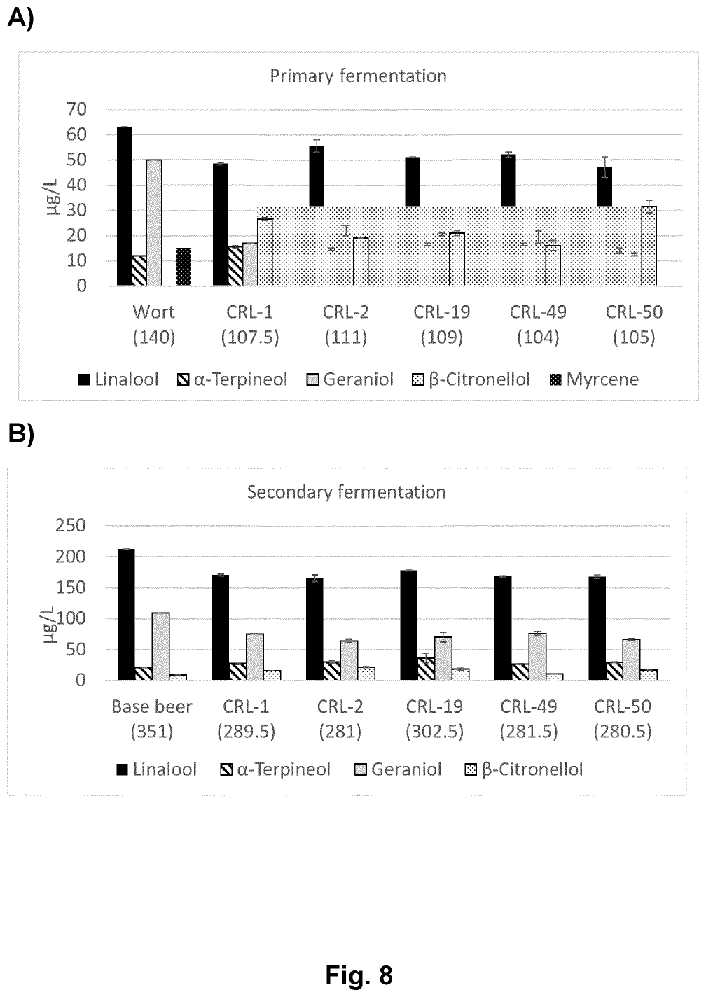

A dry-hopped commercial beer with 1.2% glucose added was inoculated with the respective strain, re-sealed in the ANKOM system and allowed to re-ferment for 14 days. At this point the glucose was depleted in all cases as shown by the CO₂ accumulation curves and between 6.8 and 7.3% alcohol had been produced. The absolute amounts of monoterpene alcohols was higher in the secondary fermentations compared to the primary fermentations due to the dry hopping applied into the primary beer (FIG. 8). However, bioconversion of monoterpene alcohols occurred to similar extents as was seen in the primary fermentation. For example in both the primary fermentation and secondary fermentation ca. 25 microgram/L geraniol was converted.

1. A method of producing a malt and/or cereal based beverage, said method comprising the steps of
i) providing an aqueous extract of malt and/or cereal kernels
ii) providing a Dekkera yeast strain, wherein said yeast strain is not capable of converting more than 25% of p-coumaric acid into 4-ethylphenol when incubated in an aqueous solution comprising p-coumaric acid
iii) fermenting said aqueous extract with said yeast thereby obtaining said malt and/or cereal based beverage.

2. The method according to claim 1, wherein said yeast strain is not capable of converting more than 25%, not more than 20%, not more than 15%, not more than 10%, not more than 5%, or not more than 1% of the p-coumaric acid present in the aqueous solution into 4-vinylphenol.

3. The method according to claim 1, wherein said yeast strain has the genotype I and/or the genotype II:
I: comprising a mutation in or a deletion of the gene encoding PAD
II: comprising a mutation in or a deletion of the gene encoding SOD.

4. The method according to claim 1, wherein the yeast strain is a Dekkera anomalus yeast strain having the genotype I: comprising a mutation in or a deletion of the gene encoding DaPAD1 of SEQ ID NO:2 or a functional homologue thereof having at least 80%, at least 90%, or at least 95% sequence identity herewith.

5. The method according to claim 1, wherein the yeast strain is of the species Dekkera anomalus, and said yeast strain comprises a mutant DaPAD1 gene encoding a mutant DaPAD1 protein lacking at least 50 amino acids, at least 70 amino acids, at least 100 amino acids, or at least 150 amino acids of SEQ ID NO:2.

6. The method according to claim 1, wherein the yeast strain is a Dekkera bruxellensis yeast strain having genotype I:
I: comprising a mutation in or a deletion of the gene encoding DbPAD2 of SEQ ID NO:6 or a functional homologue thereof having at least 80%, at least 90%, or at least 95% sequence identity herewith.

7. The method according to claim 1, wherein the yeast strain is of the species Dekkera bruxellensis, and said yeast strain comprises a mutant DbPAD2 gene encoding a mutant DbPAD2 protein lacking at least 50 amino acids, at least 70 amino acids, at least 100 amino acids, or at least 150 amino acids of SEQ ID NO:6.

8. The method according to claim 1, wherein the yeast strain:
i) is of the species Dekkera anomalus, and said yeast strain comprises a mutant DaSOD gene encoding a mutant DaSOD protein lacking at least the 50 most C-terminal amino acids, at least the 100 most C-terminal amino acids, or at least the 150 most C-terminal amino acids of SEQ ID NO: 4; or
ii) is of the species Dekkera bruxellensis, and said yeast strain carries a mutation in the DbSOD gene resulting in a mutant DbSOD gene encoding a mutant DbSOD protein lacking one or more of the amino acids of SEQ ID NO:8.

9. The method according to claim 1, wherein said yeast strain is not capable of converting more than 20%, not more than 15%, not more than 10%, not more than 5%, or not more than 1%, of the p-coumaric acid present in the aqueous extract into 4-ethylphenol.

10. The method according to claim 1, wherein said yeast strain is not capable of converting more than 25%, not more than 20%, such as not more than 15%, not more than 10%, not more than 5%, or not more than 1% of the ferulic acid present in the aqueous extract into 4-ethylguaiacol.

11. The method according to claim 1, wherein said yeast strain is not capable of converting more than 25%, not more than 20%, not more than 15%, not more than 10%, or not more than 5%, not more than 1% of the ferulic acid present in the aqueous solution into 4-vinylguaiacol.

12. The method according to claim 1, wherein said malt and/or cereal based beverage comprises less than 0.5 mg/L of 4-ethylphenol, less than 0.3 mg/L, or less than 0.1 mg/L 4-ethylphenol.

13. The method according to claim 1, wherein said malt and/or cereal based beverage comprises less than 1 mg/L of 4-ethylguaiacol, less than 0.8 mg/L, less than 0.6 mg/L, or less than 0.5 mg/L of 4-ethylguaiacol.

14. The method according to claim 1, wherein the aqueous extract is wort or a fermented malt and/or cereal based beverage.

15. The method according to claim 1, wherein the yeast strain is not capable of utilizing more than 2% of the maltose present in the aqueous extract.

16. The method according to claim 1, wherein said yeast further carries a mutation in or a deletion of one or more of the following genes:
c. MTRA1, wherein the MTRA1 gene encodes a MTRA1 protein of SEQ ID NO:10 or 16 or a functional homolog thereof sharing at least 95% sequence identity therewith
d. MTRA2, wherein the MTRA2 gene encodes a MTRA2 protein of SEQ ID NO:14 or 20 or a functional homolog thereof sharing at least 95% sequence identity therewith;
e. ISOM(1), wherein the ISOM(1) gene encodes a ISOM(1) protein of SEQ ID NO:22 or a functional homolog thereof sharing at least 95% sequence identity therewith;
f. ISOM, wherein the ISOM gene encodes a ISOM protein of SEQ ID NO:12 or a functional homolog thereof sharing at least 95% sequence identity therewith;
g. ISOM(2) wherein the ISOM(2) gene encodes a ISOM(2) protein of SEQ ID NO:18 or a functional homolog thereof sharing at least 95% sequence identity therewith;
h. MTRA3, wherein the MTRA3 gene encodes a MTRA3 protein of SEQ ID NO:26 or a functional homolog thereof sharing at least 95% sequence identity therewith;
i. MTRA4, wherein the MTRA4 gene encodes a MTRA4 protein of SEQ ID NO:28 or a functional homolog thereof sharing at least 95% sequence identity therewith;
j. MTRA5, wherein the MTRA5 gene encodes a ISOM protein of SEQ ID NO:30 or a functional homolog thereof sharing at least 95% sequence identity therewith;
k. MTRA6, wherein the MTRA6 gene encodes a MTRA6 protein of SEQ ID NO:32 or a functional homolog thereof sharing at least 95% sequence identity therewith.

17. A Dekkera yeast strain, wherein said yeast strain is not capable of converting more than 25% of p-coumaric acid into 4-ethylphenol when incubated in an aqueous solution comprising p-coumaric acid.

18. The yeast strain according to claim 17, wherein the yeast strain is as defined in claim 1.

19. A beverage prepared by the method according to claim 1.

20. A method of producing a malt and/or cereal based beverage comprising less than 3% ethanol, said method comprising the steps of
i) providing an aqueous extract of malt and/or cereal kernels
ii) providing a Dekkera yeast strain, wherein said yeast strain is not capable of utilizing more than 2% maltose when incubated at 25° C. for 10 days in an aqueous solution comprising in the range of 40 to 100 g/kg maltose and in the range of 8 to 50 g/kg glucose,
iii) fermenting said aqueous extract with said yeast
thereby obtaining said malt and/or cereal based beverage.
